(19) United States
(12) Patent Application Publication
(10) Pub. No.: US 2012/0322152 A1
(43) Pub. Date: Dec. 20, 2012

(54) Culture Additives To Boost Stem Cell Proliferation And Differentiation Response

(76) Inventors: Michael Raghunath, Singapore (SG); Hui Li Loe, Singapore (SG); Anna Blocki, Singapore (SG)

(21) Appl. No.: 13/581,680

(22) PCT Filed: Mar. 2, 2011
(86) PCT No.: PCT/SG2011/000081

§ 371 Date: Aug. 29, 2012

Related U.S. Application Data
(60) Provisional application No. 61/309,509, filed on Mar. 2, 2010.

(51) Int. Cl. C12N 5/0775; C12N 5/0793; C12N 5/0789; C12N 5/077; C12N 5/071; C12N 5/0735

(52) U.S. Cl. 435/369; 435/377; 435/366; 435/372; 435/404; 435/395

(57) ABSTRACT

The invention relates to methods of culturing and/or proliferating stem cells such as progenitor cells, multipotent and induced pluripotent stem (IPS) cells. More particularly, the invention relates to the use of macromolecular crowding created using carbohydrate-based macromolecule to promote the growth of the stem cells in an ex vivo culture, while preserving their multipotentiality.

### RELATED APPLICATION(S)

This application claims the benefit of U.S. Provisional Application No. 61/309,509, filed on Mar. 2, 2010. The entire teachings of the above application are incorporated herein by reference.

### BACKGROUND OF THE INVENTION

In all tissues of the body there is a sub-population of adult stem and progenitor cells. These multipotent cells are recruited and activated to take part in tissue regeneration. Adult stem cells are a promising resource for therapy, but their numbers are very low and they need extensive propagation in vitro to be of therapeutic use. When these cells are cultured ex-vivo it has proven difficult to recreate their natural microenvironment, which is thought to be a sum of signals from interactions with the extracellular matrix, soluble macromolecules, neighboring cells and the hormonal status of the microenvironment. Therefore, regenerative therapies using adult stem cells are still hampered by the limited number of available cells and the fact that their expansion in vitro, necessary to attain therapeutic numbers, compromises their differentiation and proliferative potential. The same applies for induced pluripotent stem cells cells, that are derived from somatic differentiated cells that are reprogrammed into pluripotency.

Thus, a need exists for methods and media compositions that help to overcome the problems encountered in ex vivo stem cell culture.

### SUMMARY OF THE INVENTION

In the ex vivo culture of stem cells a major disadvantage arises from the fact that a change in the microenvironment from that normally found in the naive stem cell niche results in the spontaneous differentiation of stern cells in culture. The microenvironment of the stem cell niche is a complex pattern of signals from interactions with specific components of the extracellular matrix (ECM), neighbouring cells and growth factors.

The biochemical cues directing the fate of a cell in the niche are composed of growth factors and their co-factors. The microenvironment is dominated by the presence of macromolecular crowding. It has been found that re-emulation of a crowded environment in differentiated cells stimulates and induces extracellular matrix deposition, thus augmenting pericellular crowding (Lareu, R R., et al., Tissue Engineering, 13(2):385-391 (2007a); Lareu, R R, et al., FEBS Lett, 581(14):2709-2714 (2007b)). While these effects were achieved with negatively charged macromolecular crowders, proliferation was usually slowed down.

As shown herein, addition of macromolecular crowders to a culture of cells (e.g., stem and progenitor cells such as bone marrow derived adult human mesenchymal stem cells, fibrocytes) enhances or optimizes the culture conditions for propagation and differentiation of these cells.

Specifically, it has been demonstrated that the presence of Ficoll cocktail increases the proliferation without shortening the lifespan of hMSCs compared to control cultures. This increase in proliferation was not associated with a relative loss of stem cells, as measured by colony forming units assays, expression of stem cell markers and multipotentiality assays.

Accordingly, a first aspect of the invention relates to a method of proliferating stem cells and progenitor cells comprising the addition of macromolecular crowders to ex vivo culture of stem cells.

Thus, macromolecular crowders such as the Ficoll cocktail are valuable tools for the ex vivo propagation of stem cells, maintenance of “stemness”, ability to differentiated and thus help unlock the potential use of stem cells for therapy and tissue repair.

Human mesenchymal stem cells (MSCs) and other stem cells are a crucial cell source for tissue regeneration and for therapeutic applications. For example, MSCs isolated from the adult bone-marrow are capable of differentiating into multiple lineages such as adipocytes, osteoblasts and chondrocytes (Pittenger, M F, et al., Science, 5411:143-147 (1997)). The maintenance of their phenotype or differentiation are governed by specific cues within each unique local microenvironment that arise from secreted factors, neighboring cells and the extracellular matrix (ECM). When administered back to the human body they home into niches like the bone-marrow and become quiescent or attain the phenotype of the tissue microenvironment into which they are introduced (Lemischka, I R et al., Nature, 425:778-779 (2003)). These microenvironments are complex and composed of a multitude of interacting extracellular matrix (ECM) components that carry ligands which in turn present stored growth factors like fibroblast growth factor 2 (FGF2) and transforming growth factor-beta (TGF-β). Besides specific growth factor receptors, these biochemical cues are sensed by ECM receptors such as integrins, membrane proteoglycans and non-integrin receptors (discoidins).

In addition to being very complex, the physiological tissue microenvironment is also crowded and exhibits an Excluded Volume Effect (EVE) that is generated by the mutual impenetrability of solute molecules. This situation is maintained in both the intra and extracellular space by the high total concentration of ECM macromolecules which occupy vast parts of a given volume and exclude like-sized molecules by steric exclusion and additional electrostatic repulsion. This high level of fractional occupancy has been shown to greatly influence equilibria and rates of biochemical reactions that depend on non-covalent associations and/or conformational changes, such as protein and nucleic acid synthesis, intermediary metabolism, cell signaling and gene expression (Ellis J R, Curr Opin Struct Biol, 1:114-119 (2001)). The in vitro addition of various polymeric macromolecules exert the excluded volume effect (EVE), speed-up specific enzymatic steps required for collagen deposition and result in the deposition of an ECM that contained 20-30 fold higher concentration of collagen possessing the correct crosslinks. (Lareu, R R, et al., FEBS Letters, 14:2709-2714 (2007)).

Although many of the intracellular events that occur during stem cell differentiation are now understood, it is not generally appreciated that a key step involves the remodeling of the matrix of naïve MSCs to generate the matured and lineage-specific matrix of the differentiated phenotypes (Lilla J, et al., Am J Pathol, 5:1551-1554 (2002)) which has been shown to provide positive feedback and promote differentiation of many cell types (Hay, Cellular Biology of Extracellular Matrix, ed. E. D. Hay, New York, Plenum (1991)). For example, it has been demonstrated that the ECM is involved in initiating the transcription of differentiation-specific genes in differentiated cells (Caron J M, Mol Cell Biol, 3:1239-1243 (1990), DiPersio, C M, Mol Cell Biol, 9:4405-4414 (1991)), and the remodelled matrix is a key player in the adipogenic developmental process enabling differentiating cells to undergo morphological and cytoskeletal reorganizations, which are required for induction of expression of lipogenic mRNAs (Lilla J, et al., Am J Pathol, 5:1551-1554 (2002)).

As described herein, it was observed that EVE generated by application of one or more macromolecules (e.g., the negatively charged Dextran Sulfate 500 (500 kDa), neutral polyanions, Ficoll 70 (70 kDa), Ficoll 400 (400 kDa)) during adipogenic and osteogenic induction of MSCs was able to increase the efficiency of differentiation (speed) and enabled them to attain a more matured phenotype. It was hypothesized that this was due to EVE speeding up the numerous enzymatic processes involved in matrix remodeling that occurs during differentiation thus generating a more favourable microenvironment that resembles the matured niche.

Accordingly, in one aspect, the invention is directed to a method of culturing stem cells comprising contacting the stem cells with one or more carbohydrate-based macromolecules, thereby producing a cell culture. The cell culture is maintained under conditions in which the stem cells proliferate, thereby culturing the stem cells. In a particular aspect, the stem cells are contacted with a mixture of carbohydrate-based macromolecules comprising Ficoll™ 70 and Ficoll™ 400 and the stem cells are mesenchymal stem cells.

In another aspect, the invention is directed to a method of producing an extracellular matrix comprising contacting fibroblasts with one or more carbohydrate-based macromolecules, thereby producing a cell culture. The cell culture is maintained under conditions in which the fibroblasts proliferate and produce an extracellular matrix. In a particular aspect, the method can further comprise contacting the cell culture with one or more agents that lyse the cells, thereby producing a cell-free extracellular matrix.

A cell-free extracellular matrix produced by the methods described herein is also encompassed by the invention.

In another aspect, the invention is directed to a method of culturing stem cells comprising contacting the stem cells with a cell free extracellular matrix produced as described herein, thereby producing a stem cell culture. The stem cell culture is maintained under conditions in which the stem cells proliferate, thereby culturing the stem cells.

In yet another aspect, the invention is directed to a kit comprising a cell-free extract produced using the methods described herein and, optionally, instructions for use of the extracellular matrix.

### BRIEF DESCRIPTION OF THE DRAWINGS

FIGS. 1A-1C show the effect of macromolecular crowding on human mesenchymal stem cell proliferation in short term cultures at different seeding densities 5000, 10,000, 25000 and 50000 cells per well (1.8 cm²) for 2, 4 and 7 days in culture, respectively.

FIGS. 2A-2B show the long term effect of macromolecular crowding on the population doubling rate of human mesenchymal stem cell seeded at (FIG. 2A) 5000 cells per well and (FIG. 2B) 1000 cells per well (1.8 cm²).

FIGS. 3A-3E illustrate the long-term effect of macromolecular crowding on the cumulative population doubling and fold-change of human mesenchymal stem cell seeded at (FIG. 3A, C) 5000 cells per well, (FIG. 3B, E) 1000 cells per well and 2000 cells per well (FIG. 3D) (well area=1.8 cm²).

FIG. 4A-4B illustrate the projected cell numbers that would be obtained under macromolecular crowding if they were propagated under current density of (FIG. 4A) 5000 cells per well and (FIG. 4B) 1000 cells per well (1.8 cm²) after 70 days and 28 days respectively.

FIG. 5 shows the effects of macromolecular crowding on the colony forming unit (CFU) ability of the human mesenchymal stem cells after being cultivated in the absence (−FC) or presence of crowders (+FC) for 56 days (d56) and 70 days (d70).

FIG. 6 shows the effect of macromolecular crowding on human mesenchymal stem cell surface marker expression.

FIG. 7 shows the effect of macromolecular crowding for pre-propagation of human mesenchymal stem cells prior to adipogenic differentiation and demonstrating preserved differentiation ability on a higher level.

FIG. 8 shows the effect of macromolecular crowding on the relative telomere length of the human mesenchymal stem cells during the course of ex vivo cell culture.

FIG. 9 illustrates the effect of macromolecular crowding during the first 3 days of a seven day culture on the cell numbers of fibrocytes growing on fibronectin-coated in 24-well plate wells.

FIG. 10 shows phase contrast photogaphs (10× magnification) of fibrocytes growing on fibronectin-coated dishes for seven days

FIG. 11 shows flow cytometry analysis for stem cell marker CD105, leukocyte marker CD45, hematopoetic stem cell marker CD34 and for monocyte marker CD14, all markers for fibrocytes, after one week of culture.

FIG. 12 shows flow cytometric analysis of a cell population cell colony coexpressing fibrocyte markers after 1 week of culture.

FIG. 13 shows adherent cytometry of in situ MSC monolayers after 10 days in culture under macromolecular crowding using a ternary mixture of Ficoll and PVP.

FIGS. 14A-14D show that macromolecular crowding enhances adipogenesis by increasing the amount of cytoplasmic lipid droplet accumulation (the effect of macromolecular crowding (MMC) on lipid droplet formation). (14A) & (14B): Nile red staining of lipid droplets in hBMSC cells at passage 5 after induction at 100% and 25% strength induction in the absence or presence of MMC for 7 and 14 days and viewed at 10× magnification. (14C) & (14D): Comparisons in differences in lipid droplet accumulation was quantified with a Metamorph imaging software at 2× after staining with Nile Red and normalized to nuclei via DAPI staining. With crowding, an increased fold change of 1.68±0.79 and 7.4±0.89 was observed after 7 days of induction at 25% and 100% strength, respectively; a similar trend at 2.95±0.14 and 2.2±0.13 was observed after 14 days of induction.

FIGS. 15A-15D show the effects of macromolecular crowding on the population percentage of hMSCs that undergo adipogenesis. Results of flow cytometric analysis on Nile Red staining of hBMSCs cultures at passage 4 and 8 after adipogenic induction by a commercial induction kit at half- or full-strength kit in the absence or presence of MMC (21 days). (15A) & (15C) Quantification of lipid accumulation with FACScan flow cytometry based on Nile red staining. Analysis was presented using a forward scatter (FSC) versus fluorescence in the FL2 channel, where the percentage of adipocytes was calculated based on the cells stained with high FL2 values (>102). (15B) & (15D) An increased fold change was observed under both induction condition in the presence of MMC. A positive fold change was observed in the percentage of events exhibiting higher FL2 for half-strength induction with crowding compared to standard strength induction in the absence of crowding, this indicates a more effective use of less induction ingredients with crowding in causing adipogenesis.

FIGS. 16A-16D show that crowding increased the expression of key adipogenic genes, PPARg and aP2. A decrease in collagen IV gene expression was observed although this is accompanied by an increase in actual protein deposited.

FIG. 17 shows that crowding increased the deposition of collagen IV in the cell layer (western blot), this is accompanied by decreased expression of col IV mRNA. Results of western blot analysis of hBMSCs cultures at passage 5 after adipogenic induction at 25% or 100% in the absence or presence of crowding after 7 or 14 days. An increase in the collagen IV band with crowding at both induction strength with the addition of crowding was observed.

FIG. 18 shows effects of macromolecular crowding on the amount of calcification in MSC monolayer cultures. Alizarin red staining of osteogenically induced MSC monolayers in the absence or presence of crowding over 4 weeks indicates that crowding dramatically enhances the amount of calcification in monolayers in week 3 and week 4, and thus, the extent of differentiation.

FIG. 19 shows effects of macromolecular crowding on the expression level of lipid rafts on monolayer. Cholera Toxin B-FITC staining of monolayers cultured for 7 days in the absence or presence of crowding indicates that crowding dramatically enhances the amount of lipid rafts expressed in the monolayer.

FIG. 20 shows adipogenesis at passage 4. Human bMSC (at P3): propagation under Ficoll mix gives significant increase of cell number in fold change. At passage +4: 244% increase in cell numbers was noted. Cells at this passage showed significantly increased adipogenesis when chemically induced.

FIG. 21 shows increased proliferation rate under mixed macromolecular crowding (two or three macromolecular species).

FIGS. 22A-22D show macromolecular crowding enhances adipogenesis of hMSCs. (FIG. 22A) Naïve MSCs at passage 4 were induced to undergo adipogenesis in the absence (ctrl) or presence of macromolecular crowding (MMC) for 21 days, and (FIG. 22B) stained with nile red; assessed via in-situ adherent cytometry to quantify area of nile red staining per cell; via (FIG. 22C) flow activated cytometry to assess percentage of the cells that had differentiated; and (FIG. 22D) by real-time PCR for the expression of the early adipogenic marker PPAR-gamma, late marker leptin and ECM marker Collagen IV. (n=3; error bars are ±s.d.; ** P<0.01; * P<0.05).

FIGS. 23A-23B show macromolecular crowding increases extracellular matrix deposition and remodeling by adipogenically induced MSCs and ligands sequestered.

FIG. 23A shows immunochemical staining of cell layers for collagen IV (Col IV), fibronectin (FN) and heparan sulfate at 10× magnification in adipogenically induced hMSC monolayers. Crowding (+MMC) is seen to enhance the deposition of collagen IV and heparan sulfate indicating a more enriched matrix. There is less fibronectin seen in the crowded monolayer which is in agreement with enhanced degradation during the adipogenic matrix remodeling process.

FIG. 23B (i) shows immunoblotted for cleaved procollagen C-propeptide (C3; red arrows) in medium samples from adipogenically induced monolayers after 4 days in culture, with or without MMC. MMC resulted in a more efficient processing of the procollagen as seen in the decrease of the procollagen bands and an increase in the procollagen C-propeptide detectable in the media as visualized in the darker band. (ii) MMC increased C3 by 3 fold based on densitometry.

FIGS. 24A-24C show different matrices affect potency of adipogenic induction cocktail over 21 days. Matrices were deposited by naïve mesenchymal stem cells (hMSCs) and adipogenically-induced hMSCs (adip) in the absence (−MMC) and presence of crowder (+MMC); decellularised then reseeded with naïve hMSCs which were induced to undergo adipogenesis for 3 weeks (no crowding). As a control, cells were seeded on tissue culture polystyrene (TCPS) and adipogenically induced for 3 weeks.

FIG. 24A shows adipocyte matrix deposited under MMC shows the highest amount of nile red staining. MSCs on TCPS and induced adipogenically positively stain for nile red, demonstrating the capacity for the cells to differentiate.

FIG. 24B shows reseeded monolayers are assessed via in-situ adherent cytometry to quantify area of nile red staining per cell. hMSC matrices show a restraining effect with reseeded hMSCs expressing less lipid droplets than the positive benchmark (Induced on TCPS). Adipocyte matrix results in more staining than TCPS and the effect is potentiated by MMC; 40% vs 7%.

FIG. 24C shows real-time PCR was used to assess the expression of the early adipogenic marker PPAR-γ in the reseeded monolayers (n=3; error bars are ±s.d). hMSC matrices show a restraining effect with reseeded hMSCs expressing less PPAR-γ than the positive benchmark (Induced on TCPS). Adipocyte matrix that was deposited under crowding results in 33% more PPAR-γ mRNA expression than TCPS.

FIGS. 25A-25D show the adipogenic property of decellularised adipocyte extracellular matrices is sensitive to salt-elution. Decellularised matrices were deposited by fully differentiated adipocytes (adip) in the absence (−) and presence (+) of macromolecular crowding (MMC). One group of matrices were additionally treated with 0.5 M NaCl (+salt elution). Salt-treated and non-treated matrices were then re-seeded with naïve hMSCs. After 3 weeks in basal media, cells were assessed for features of adipogenic differentiation.

FIG. 25A shows adipocyte matrix deposited under MMC shows the highest amount of nile red staining. As a control, cells were adipogenically induced (or not) for 3 weeks on tissue culture polystyrene (TCPS), demonstrating the capacity for the cells to differentiate.

FIG. 25B shows reseeded monolayers are assessed via in-situ adherent cytometry to quantify area of nile red staining per cell. Adipocyte matrix results in ˜5-10% nile red staining compared to those on TCPS. This inductive property of the matrix was lost upon salt elution. (n=3; error bars are ±s.d; ** P<0.01; * P<0.05).

FIG. 25C shows real-time PCR was used to assess the expression of the early adipogenic marker PPAR-γ in the reseeded monolayers (n=3; error bars are ±s.d). Adipocyte matrix results in 5% PPAR-γ mRNA expression of that on TCPS. This inductive property of the matrix was lost upon salt elution. (n=3; error bars are ±s.d; ** P<0.01; * P<0.05).

FIG. 25D shows real-time PCR was used to assess the expression of the late adipogenic marker leptin in the reseeded monolayers (n=3; error bars are ±s.d). Adipocyte matrix results in 50-70% PPAR-γ mRNA expression of that on TCPS. This inductive property of the matrix was lost upon salt elution. (n=3; error bars are ±s.d; ** P<0.01; * P<0.05).

FIGS. 26A-26B show macromolecular crowding speeds up the appearance of cytoplasmic lipid droplets and elevates expression of PPARγ during early adipogenesis of hMSCs. (FIG. 26A) Oil red O staining of monolayers at 10× magnification showing cytoplasmic lipid droplets (stained red) at days 2,4 and 7 post-induction in the absence and presence of MMC. (FIG. 26B) Relative mRNA expression levels of PPARg at days 2, 4 and 7 post-induction in the absence and presence of MMC (n=3; error bars are ±s.d.; ** P<0.01; * P<0.05).

FIGS. 27A-27B show ECM Deposited in the presence of MMC more stable in the face of detergent Lysis.

FIG. 27A shows immunochemical staining of cell layers for ECM key proteins—collagen IV (col IV), collagen I (Col I) and fibronectin (FN and cellular components—ER, nuclei and actin; at 10× magnification. ECM proteins deposited under MMC are more resistant and retained during detergent lysis. Lysis is successful at decellularising ECM.

FIG. 27B shows protein gel analysis confirms the presence of more proteins retained under MMC (+MMC) post-lysis.

FIG. 28 shows characterisation of matrices. Immunofluorescence stainings showing the presence of several ECM proteins in DxSDOC and DxSDOCDOC matrices. Bar 100 μm.

FIGS. 29A-29B show hESCs propagated on NoDxSDOC, DxSNP40 and FcNP40 matrices differentiated or did not attach. (29A) Population doublings of hESCs propagated using on NoDxSDOC, DxSNP40 and FcNP40. (29B) Phase contrast images showing hESCs' differentiated morphology and non-attachment during propagation. Bar 500 μm.

FIGS. 30A-30B show hESCs propagated on Matrigel, DxSDOC or DxDOCDOC matrices for up to 20 passages. (30A) Population doublings of hESCs propagated using either collagenase IV or dispase on Matrigel, DxSDOC or DxSDOCDOC. (30B) Phase contrast images showing hESCs' morphology during propagation. Bar 500 μm.

FIGS. 31A-31C show hESCs propagated on DxSDOC or DxSDOCDOC matrices for 18 passages retained karyotypic stability and differentiation capacity. (31A) Karyograms of hESCs propagated on Matrigel, DxSDOC or DxSDOCDOC using collagenase IV or dispase. (31B) hESCs were positive for neural marker Tubulin when induced to undergo neural differentiation, showing that hESCs retained neural differentiation capabilities after long term passaging. Bar 100 μm. (31C) hESCs formed teratomas with differentiated structures representative of tissues of the three germ layers, indicating the retention of pluripotency. Bar 50 μm.

FIG. 32 shows flow cytometry of SSEA-3 and SSEA-4 expression in of hESCs showing expression of pluripotency markers in hESCs propagated on Matrigel, DxSDOC or DxSDOCDOC matrices for 20 passages.

FIG. 33 shows adherent immunofluorescence analysis for Oct 4, SSEA 4, TRA-1-60, TRA-1-81 and SSEA-3 expression in hESCs (Bar 100 μm) showing expression of pluripotency markers in hESCs propagated on Matrigel, DxSDOC or DxSDOCDOC matrices for 20 passages.

FIG. 34 shows complete decellularization to obtain matrices. Immunofluorescence stainings for intracellular structures reveals the removal of ER, F-actin and nuclei after DOC, DOCDOC and NP40 lysis. Bar 100 μm.

FIG. 35 shows hESCs cultured on ECM made by 293T cells and NT2 cells differentiated within 12 passages. The ECM were made by crowding 293T cells and NT2 cells using DxS for 3 days and subjecting the ECM to detergent lysis DOC treatment.

FIG. 36 shows (a) As hMSCs differentiate into the adipogenic lineage, (b) they assume a more cuboidal morphology followed by (c) accumulation of lipid droplets within their cytoplasm.

FIG. 37 shows adipogenic differentiation was stimulated via three cycles of induction medium (IM)/maintenance medium (MM) either at full- or half-strength IM. Half-strength adipogenic IM is 1:1 ratio of IM and MM. Non-induced controls were maintained in MM on the same schedule. At three time points corresponding to the end of each induction cycle, monolayers in 24-well plates were assessed via FACS, adherent cytometry and RT-PCR. Lipid droplets were stained with Nile Red (Greenspan, P., et al., J Cell Bio. 100:965-973 (1985)) then analyzed with FACS to count the number of differentiated MSCs and imaged with adherent cytometry to measure lipid accumulation.

FIG. 38 shows hMSCs (p8) after 21 days induction, stained with Nile Red (2× mag). Larger and more numerous lipid droplets were observed in the presence of EVE at half-strength (e) and full strength (f) compared to controls without EVE (b,c). Negligible staining was observed with no induction (a,d).

FIG. 39 shows automated adherent cytometry (Chen, C., et al., unpublished) quantified in-situ area of Nile Red stained lipid droplets, normalized to cell number of induced hMSCs (p8). EVE was observed to increase lipid accumulation consistently over 3 weeks at both half- and standard induction strength. EVE also enabled more effective use of less induction ingredients at half-strength (+EVE) compared to standard strength (−EVE). Negligible staining was observed with no induction.

FIG. 40 shows FACS analysis of hMSCs stained with Nile Red at late passage (p8) after 21 days incubation in maintenance media (a,d), half-strength induction (b,e) and full strength induction (c,f) in the absence (a,b,c) or presence (d,e,f) of EVE. Cell population in upper right quadrant are considered positive for adipogenesis. EVE increased the percentage of hMSCs undergoing adipogenesis by 1.8 fold and 2 fold at half- and standard strength respectively. EVE also enabled more effective use of less induction ingredients at half-strength (+EVE) compared to standard strength (−EVE) by 1.7 fold.

FIG. 41 is a schematic of an experimental overview.

FIG. 42 shows immunocytochemical staining for collagen I & IV of monolayers after 3 and 7 days of adipogenic induction (10× mag). With crowding; we observed enhanced deposition of collagen IV and enhanced reticular structuring of collagen I; which are key steps during adipogenic matrix remodeling.

FIG. 43 shows automated adherent cytometry (C. Z. C. Chen, et al., (2009) Brit. J Pharmacol (in press)) quantified in-situ area of Nile Red stained lipid droplets, normalized to cell number of induced MSCs. Crowding was observed to increase cytoplasmic lipid accumulation after 3 weeks induction at induction strengths of 50% (E) and 100% (F) compared to uncrowded (B&C). Negligible staining was observed with no induction (A&D).

FIG. 44 shows FACS analysis of Nile Red stained hMSCs after 21 days incubation at 0% (A,D), 50% (B,E) and 100% induction (C,F) in the absence (A,B,C) or the presence (D,E,F) of crowding. Cell population in upper right quadrant are considered positive for adipogenesis. Crowding increased the percentage of hMSCs undergoing adipogenesis by 1.8 fold and 2 with induction at 50% and 100% respectively.

FIG. 45 shows quantitative PCR for aP2 gene expression 7 days post-adipogenic induction at 20% and 100%. With crowding we observed, a 2.4 fold increase in aP2 expression at 20% induction strength and 2.6 fold increase at 100% induction.

FIG. 46 shows adherent cytometry of MSCs seede on three different substrates—plastic, matrix deposited (over 3 weeks) by adipocytes in the absence (−crowding) or presence of crowding (+crowding); followed by 100% induction for 10 days. A 4 fold increase in cytoplasmic limid accumulation was observed with monolayers seeded on matrix deposited in the presence of crowding (c) compared with those on seede on matrix deposited in the absence of crowding (b).

### DETAILED DESCRIPTION OF THE INVENTION

Human adult stem cells (from bone marrow and many other sources) have been utilized for in vivo tissue engineering to generate novel clinical modalities for autologous repair or for ex vivo culture and re-administration into patients. Currently, their use is limited by the attenuation of their differentiation potential following extensive ex vivo expansion. The maintenance of their phenotype or differentiation are governed by specific cues within each unique local microenvironment that arise from secreted factors, neighboring cells and the extracellular matrix (ECM). Described herein are methods which use Macromolecular Crowding (MMC) to recapitulate the crowded physiological environment in vitro using a cocktail e.g., a neutral polyanion cocktail. As shown herein, MMC enhanced proliferation of undifferentiated stem cells and enhanced differentiation of stem cells upon lineage-specific induction. The methods described herein were exemplified using a polyanion cocktail comprising Ficoll™ 70 (Fc70; 70 kDA), Ficoll™ 400 (Fc400; 400 kDa), neutral dextran 670 (Dx670; 670 kDa), polyvinyl pyrrolidone 360 (PVP; 360 kDa) and combinations thereof to proliferate hBMSCs and induce adipogenic and osteogenic differentiation of the hBMSCs.

The data provided herein shows that the methods of the present invention provide a faster response as visually observed by the earlier onset of a more differentiated phenotype (adipogenic lipid droplets) at day 5 of the induction cycle. The data also show an increased proportion of hbMSCs at passage 5 post-adipogenic induction (14 d) that expressed the differentiated phenotype increased from 3.45±0.46% to 12.4±1.8% at 25% induction strength (a 3.6 fold change increase); at 100% induction population differentiated increased from 9.54±0.89 to 22.24±8.9 (a 2.3 fold change increase). In addition, a more matured phenotype was attained when hbMSCs at passage 4 were differentiated for 14 days under crowding as measured by the area of cytoplasmic lipid droplets expressed exhibited a fold change increase with crowding of 2.95±0.14 and 2.2±0.13 at 25% and 100% induction strength respectively. Also observed was an increased proliferation of stem cells prior to induction of differentiation as well as a stronger differentiation push in a higher MSC passage number (passage 8), and a strong differentiation response even in the presence of 50% or 25% strength of induction cocktail. Surprisingly, MSC's also show an enhanced proliferation rate on cell culture plastic while retaining their differentiation potential over several passages as demonstrated by the retention of STRO1 marker, preserved telomere length and differentiation potential. This is an important issue increasing MSC cell numbers as this is currently a bottleneck.

The data are provided herein derived from expression of key differentiation markers, cytometry, FACS analyses. MMC appears two work via two pathways (a) increasing the density of cell surface receptors housed in lipid rafts (b) inducing extracellular matrix deposition and accelerating its lineage-specific remodeling. Macromolecular crowding is a tool that can be utilized to generate novel tissue culture conditions for more efficient stern cell differentiation by recapitulating the in vivo physiological environment and enhanced deposition of key ECM proteins involved in the differentiation pathway.

Accordingly, in one aspect, the invention is directed to a method of culturing stem cells comprising contacting the stem cells with one or more carbohydrate-based macromolecules, thereby producing a cell culture. The cell culture is maintained under conditions in which the stem cells proliferate, thereby culturing the stem cells.

The methods described herein can be used to culture a variety of stem cell-populations. As is known to those of skill in the art, stem cells include embryonic stem cells, adult stem cells (e.g., hematopoietic stem cells, bone marrow stromal stem cells (mesenchymal stem cells), neural stem cells, epithelial stem cells, blood-borne progenitor cells such as fibrocytes and induced pluripotent stem cells any of which can be used in the methods described herein. Methods of obtaining stem cells are also well known to those of skill in the art. In addition, stem cells can be derived from a variety of sources. In particular aspects, the stem cells are mammalian stem cells (e.g., primate, canine, feline, bovine, murine, and the like). In a particular aspect, the stem cells are human stem cells.

Due to their capacity to form cartilage, bone, fat and other connective tissue, human mesenchymal stem cells (hMSCs) and blood-borne mesenchymal progenitor cells, also known as fibrocytes or reprogrammed monocytes (Φ-monocytes, MOMC, PCMO) as reviewed by Seta and Kuwana, Keio J Med, 56(2):41 (2007), constitute an exciting prospect for cell-based therapy in regenerating diseased or injured tissues. These adult stem cells can be readily purified from a small volume of bone marrow aspirates or peripheral blood respectively, and expanded in vitro for a limited number of population doublings (PD) before they reach replicative senescence. Induced pluripotent cells (IPS) cells are derived from progenitor/stem cells or somatic cells that are derived either from skin biopsies, liposuction material or bone marrow. Due to their capacity to form to generate cells of the red and white blood cell lineage, including platelets, hematopoietic stem cells constitute an exciting prospect for cell-based therapy in leukemia and deficiencies in regenerating diseased or injured tissues. Mesenchymal and hematopoeitic stem cells can be readily purified from a small volume of bone marrow aspirate, fibrocytes from peripheral blood, and IPS from harvested tissue, respectively, and expanded in vitro for a limited number of population doublings (PD) before they reach replicative senescence meaning growth arrest.

Fibrocytes are cells from monocyte origin which express mesenchymal and haematopoieitic markers such as (but not limited to) CD34, CD14, CD45, CD105, Collagen I and Vimentin. These cells express mesenchymal progenitor properties and are able to differentiate into adipocytes, osteoblasts, chondrocytes, endothelial cells etc.

Mesenchymal stem cells or human bone marrow stromal stem cells are pluripotent progenitor cells with the ability to generate cartilage, bone, muscle, tendon, ligament and fat. These primitive progenitors exist postnatally and exhibit stem cell characteristics, namely low incidence and extensive renewal potential. These properties in combination with their developmental plasticity have generated tremendous interest in the potential use of mesenchymal stem cells to replace damaged tissues. In essence mesenchymal stem cells could be cultured to expand their numbers then transplanted to the injured site or after seeding inion scaffolds to generate appropriate tissue constructs.

Thus, an alternative approach for skeletal, muscular, tendon and ligament repair is the selection, expansion and modulation of the appropriate progenitor cells such as osteoprogenitor cells in the case of bone.

Human bone marrow mesenchymal stem cells can be isolated and detected using selective markers indicating their potential for marrow repopulation. The panel of positive cell surface markers are defined as CD29, CD90, CD105 and CD166. Negative markers are. CD34 and CD45. These markers are found on the cell surface of mesenchymal stem cells and are an indication of their pluripotency.

Human blood-borne mesenchymal progenitor cells/fibrocytes can be isolated and detected using selective marker combinations. Fibrocytes express CD45 and CD105, Collagen I and Vimentin and CD14 and CD34 to some extent. They were also described to be positive for HLA-DR, CD11 a and b (reviewed by Bellini and Mattoli, Lab Invest, 87:858 (2007)).

Human bone marrow-derived haematopoietic stem cells can be isolated and detected using selective marker combinations such as CD34 and CD133. Additionally, haematopoietic stem cells have low levels or absent lineage committed markers like CD33, CD38 and CD71.

Human IPS can be generated by viral introduction of typical embryonic stem cell genes like Oct4, Sox2, Klf4, c-Myc and Nanog into somatic cells. They resemble embryonic stem cells in morphology and growing behavior, form dense cell colonies in vitro, differentiate into cells of all three germ layers and form teratomas, when injected into SCID mice. They can be distinguished from non-reprogrammed somatic cells by using selective pluripotency marker such as Tra-1-81, Tra-1-60, SSEA3 and SSEA4.

The stem culture method according to any aspect of the invention may relate to adult stem cells, human embryonic stem cells, induced pluripotent stem cells, in monolayer, feeder-layer culture or three dimensional cultures. Further, the method according to any aspect of the invention may further comprise any variety of additional culture medium that are used to proliferate stem cells which are well known to those of skill in the art.

In the methods of the invention, the stem cells are contacted with one or more carbohydrate-based macromolecule, also referred to herein as a crowder macromolecule or a macromolecular crowder. In one aspect, the stem cells are contacted with two or more carbohydrate-based macromolecules, referred to herein as mixed macromolecular crowding. In particular aspects, the stem cells are contacted with two, three, or four, etc. carbohydrate-based macromolecules.

The spontaneous differentiation of stem cells in culture is a result of a change in the microenvironment from that normally found in the naive stem cell niche. The stem cell niche is a sum of signals from interactions with specific components of the extracellular matrix (ECM) and neighboring cells, and the hormonal status of the microenvironment.

A characteristic of the microenvironment of all cells is the high total concentration of macromolecules. Such media are termed ‘crowded’ rather than ‘concentrated’ because, in general, no single macromolecular species occurs at high concentration but, taken together, account for a volume occupancy of 20-30% of a given specific volume. As pointed out by Ellis (2001) and Minton (2000) crowding by macromolecules has both thermodynamic and kinetic effects on the properties of other macromolecules that are not generally appreciated (Minton, A P, Curr Biol, 10(3):R97-9 (2000); Ellis, R J, Trends Biochem Sci, 26(10):597-604 (2001)). Biological macromolecules such as enzymes have evolved to function inside such crowded environments. For example, the total concentration of protein and RNA inside bacteria like E. coli is in the range of 300-400 g/l. Macromolecular crowding causes an excluded volume effect (EVE), because the most basic characteristic of crowding agents is the mutual impenetrability of all solute molecules. This nonspecific steric repulsion is always present, regardless of any other attractive or repulsive interactions that might occur between the solute molecules. Thus, crowding is an inevitable hallmark of the intracellular milieu of all carbon-based life-forms on earth (reviewed in Ellis, R J, Trends Biochem Sci, 26(10):597-604 (2001)). The effects resulting from macromolecular crowding are so large that authorities in the field state that many estimates of enzyme catalyzed reaction rates and equilibria made with uncrowded solutions in the test tube differ by orders of magnitude from those of the same reactions operating under crowded conditions within cells (Ellis, R J, Trends Biochem Sci, 26(10):597-604 2001).

Despite this knowledge, biochemists still commonly study enzymatic reactions in solutions with a total macromolecular concentration of 1-10 g/l or less, in which crowding is negligible. A particular example is the polymerase chain reaction which is performed in a diluted aqueous environment. If crowdedness in introduced into such a system emulating an intracellular environment, the kinetics shift dramatically, the reaction is accelerated, more amplicons are generated, the enzyme is heat-protected, and primer-template interactions are enhanced (Lareu, R R, et al., Biophy Biochem Res Comm, 363(1):171-177 (2007c), Harve, K S, et al., Nucleic Acids Res; epub (Oct. 23, 2009), Raghunath, M et al. WO 2008/018839 A1).

The principle of macromolecular crowding also reigns in the extracellular environment. Cells are surrounded by soluble and immobilised macromolecules which form their native microenvironment. Again, contemporary cell culture consists of placing adhering cells on a support (tissue culture plastic or other materials) or keeping them in suspension in aqueous media under conditions that do not reflect the crowded environment from which they have been originally derived. Thus, they cannot exert they physiological function to the fullest potential. In fact, it has been shown that when fibrogenic cells are grown under crowded conditions using negatively charged crowders, enzymatic steps are accelerated that control the deposition rate of collagen (Lareu, R R., et al., Tissue Engineering, 13(2):385-391 (2007a); Lareu, R R., et al., FEBS Lett, 581(14):2709-2714 (2007b)). The issue of the current artificial environment of cell culture becomes even more pressing with ex vivo stem cell culture which is necessary to propagate stem and progenitor cells derived from tissue environments to achieve large enough numbers that are therapeutically efficacious. Current stem cell culture is hampered by a sharp decrease in proliferative and differentiative capacity, and as shown herein, it is most likely that lack of crowdedness plays a role.

Macromolecular crowders in the extracellular space in culture have been shown to drive the enzymatically gated deposition of extracellular matrix. The most effective crowder species were negatively charged (Lareu, R R., et al., Tissue Engineering, 13(2):385-391 (2007a); Lareu, R R., et al., FEBS Lett, 581(14):2709-2714 (2007b)) while neutral crowders proved inefficient. In addition negatively charged macromolecular crowders like dextran sulfate and polystyrene sulfonate slowed down proliferation of differentiated cells. In contrast, and surprisingly, it was found herein that neutral macromolecular crowders drive the proliferation of stem cells and progenitor cells. The observed proliferation increase achieved according to the methods of the present invention is accompanied by the preservation of the multipotentiality of the stem cells and progenitor cells.

These findings are in contrast to prior art in which recommends negatively charged saccharide chains of glycosaminoglycans of a molecular size substantially smaller than 50 kDa, namely reach 25 kDa maximally (Nurcombe and Cool 2005, PCT/IB2006/000278; WO 2005/107772).

Thus, in a further aspect of the invention the macromolecular crowders are preferably a neutral and/or hydrophilic carbohydrate-based macromolecule.

As described herein, one or more carbohydrate-based macromolecules can be used in the methods of the invention. For example, multiple macromolecular crowders can be used in the methods of the invention and either individually added to the stem cell culture or added as a mixture or cocktail of macromolecular crowders (e.g., a binary or ternary cocktail).

Macromolecular crowders are inert macromolecules with a molecular weight above about 50 kDa. These macromolecules are carbohydrate based, and can be of spherical shape, and of neutral surface charge. The macromolecules according to the invention may have a molecular weight of from about 50 kDa to about 1000 kDa. In specific aspects, the molecular weight of the macromolecule is about 50, 100, 200, 300, 400, 500, 600, 700, 800, 900, or 1000 kDa. In a particular aspect, the organic-based macromolecule according to the invention is a carbohydrate-based hydrophilic macromolecule. For example, the carbohydrate-based macromolecule of the invention may be a polymer of glucose and/or sucrose. Particular examples of the macromolecule according to the invention include Ficoll™ 70, Ficoll™ 400, polyvinyl pyrrolidone (PVP), glycosaminoglycans, sugar chains of glycosaminoclycans, cellulose, pullulan or a mixture thereof. Specifically, the carbohydrate-based macromolecule can be Ficoll™ 70 or Ficoll™ 400, neutral dextran, PVP, pullulan or a mixture thereof. It has been demonstrated in the present invention that adding a Ficoll cocktail to mesenchymal stem cells increased proliferation of human mesenchymal stem cells. Ficoll can be obtained from commercial sources such as Amersham Biosciences as Ficoll™ 70 (Fc70; 70 kDA) under catalogue number 17-0310 and Ficoll™ 400 (Fc400; 400 kDa) under catalogue number 17-0300.

In other aspects, of the methods provided herein, the solution containing the macromolecule according to the invention may have a viscosity of less than about 2 mPa-s. For example, a viscosity of about 1.75 mPa-s, 1.5 mPa-s, 1.25 mPa-s, 1 mPa-s 0.75 mPa-s, 0.5 mPa-s, or 0.25 mPa-s.

In yet other aspects, the macromolecule can have a hydrodynamic radius range of from about 2 nm to about 50 nm.

In some aspects, the total macromolecular concentration is about 2.5-100 mg/ml, and in other aspects, about 5-90 mg/ml, about 10-80 mg/ml, about 20-70 mg/ml, about 30-60 mg/ml, about 40-50 mg/ml, and in yet other aspects about 10-40 mg/ml or about 10-62.5 mg/ml. In particular aspects, the macromolecule may be Ficoll™ 70 present at a concentration of 2.5-100 mg/ml, and/or Ficoll™ 400 at a concentration of 2.5-100 mg/ml, or a mixture thereof. In other particular aspects, the macromolecule may be Ficoll™ 70 present at a concentration of 2.5-37.5 mg/ml and/or Ficoll™ 400 at a concentration of 2.5-25 mg/ml, or a mixture thereof. In a particular aspect, the stem cells are contacted with a carbohydrate-based macromolecule comprising Ficoll™ 70 at a concentration of about 37.5 mg/ml and Ficoll™ 400 at a concentration of about 25 mg/ml.

The concentration of macromolecules for use in the present invention can also be calculated based on the volume fraction occupancy. As known to those of skill in the art, the composition of a solution containing very large molecules (macromolecules) such as polymers, is most conveniently expressed by the “volume fraction (Φ)” or “volume fraction occupancy” which is the volume of polymer used to prepare the solution divided by the sum of that volume of macromolecule and the volume of the solvent. In the methods described herein the cells are contacted with the one or more macromolecules at a biologically relevant volume fraction occupancy. In some aspect, the biologically relevant volume fraction occupancy is from about 3% to about 30%. In other aspects, the biologically relevant volume fraction occupancy is from about 5% to about 25%, from about 10% to about 20% and from about 12% to about 15%. Thus, in the methods of the invention, the volume fraction occupancy is about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%. In a particular aspect, the biologically relevant volume fraction occupancy is about 15%.

The macromolecule can be added to the stem cell culture in a variety of ways. For example, the macromolecules are added as a powder or liquid into culture medium. Preferably, the addition of the macromolecule does not significantly increase the viscosity of the cell culture medium. The medium can then be sterilized, e.g. via filtration, if desired. In one aspect, the crowding cocktail contains a combination of Fc 70 and Fc 400.

As also shown herein additional macromolecular crowders can be added to the cell culture medium. In one aspect, the additional crowder(s) is either a neutrally charged compound (e.g., PVP) or a negatively charged crowder (e.g., Dextran sulfate 500 kDa).

In particular aspects, the method comprises contacting the stem cells with at least two organic-based hydrophilic macromolecule of neutral surface change. In other aspects, the method comprises contacting the stem cells with (a) at least two organic-based hydrophilic macromolecule having a molecular weight of 50 kDa to 1000 kDa and neutral surface charge, or (b) at least one organic-based hydrophilic macromolecule having a radius range of 2 to 50 nm and neutral surface change, or (c) at least two organic-based hydrophilic macromolecule having a molecular weight of 50 kDa to 1000 kDa and neutral surface charge combined with a third organic-based hydrophilic macromolecule having a molecular weight of 50 kDa to 1000 kDa and neutral surface, or (d) at least two organic-based hydrophilic macromolecule having a molecular weight of 50 kDa to 1000 kDa and neutral surface charge combined with a third organic-based hydrophilic macromolecule having a molecular weight of 50 kDa to 1000 kDa and having a negative or neutral surface charge.

In the methods of the invention, the cell culture is maintained under conditions in which the stem cells proliferate. A variety of methods for maintaining the cell culture under conditions in which the stem cells proliferate can be used and are known to those of skill in the art. Such methods include introducing agents such as serum (e.g., fetal bovine serum (FBS) or human serum), growth factors, antibiotics and the like into the culture, maintaining the cell culture under serum starved conditions or in an expansion medium etc.

As used herein, the expression “proliferation” or “proliferating” is used in its regular meaning and relates to the expansion of cells or tissue, including cell growth and cell division.

The term “maintenance” as used herein in relation to the culture of stem cells, refers to the preservation of the “stemnness”, i.e. the multi potentiality, and the viability of said stem cells and progenitor cells in culture.

As also described herein, the cell culture can be further contacted with an agent that induces differentiation of the stem cells in culture into any number pf desired lineages such as adipocytes, osteoblasts, chondrocytes, neurons, muscle cells, blood cells and the like. That is, the method according to any aspect of the invention may comprise any kind of induction cocktail to differentiate stem cells into desired lineages. In particular the method according to any aspect of the invention may be for the induction of any lineage. Agents that can be used to induce differentiation of stem cells are known in the art and available from commercial sources.

In another aspect, the invention is also directed to a method of producing an extracellular matrix comprising contacting fibroblasts with one or more carbohydrate-based macromolecules, thereby producing a cell culture. The cell culture is maintained under conditions in which the fibroblasts proliferate and produce an extracellular matrix. In a particular aspect, the fibroblasts are human fibroblasts (e.g., Wi-38 fibroblasts). As shown herein the method can further comprise contacting the cell culture with one or more agents that lyse the cells, thereby producing a cell-free extracellular matrix. Agents that can be used to lyse the cells are known to those of skill in the art and include deoxycholate (DOC), NP40 or a combination thereof.

Another aspect of the invention is a cell-free extracellular matrix produced by the methods provided herein.

In a particular aspect, the invention is directed to a method of culturing stem cells comprising contacting the stem cells with the cell free extracellular matrix provided herein, thereby producing a stem cell culture. The stem cell culture is maintained under conditions in which the stem cells proliferate, thereby culturing the stem cells.

There is also provided a kit for stem culture for improving the efficiency, activity and/or stability of stem cells in vitro or ex vivo, the kit comprising at least two organic-based hydrophilic macromolecule of neutral surface charge, and optionally a third neutral or negatively charged macromolecule. However, the cell type to be treated is not limited to adult stem cells. In particular, the kit according to the invention may be a kit for boosting the efficiency, activity and/or stability also of differentiated cells the kit comprising (a) at least two organic-based hydrophilic macromolecule of molecular weight 50 kDa to 500 kDa and neutral surface charge, or (b) at least two organic-based hydrophilic macromolecule of radius range of 2 to 50 nm and neutral surface charge; or (c) and optionally at least one more organic-based hydrophilic macromolecule of radius range of 2 to 50 nm and neutral surface charge; or negative charge. The macromolecule used in the kit of the invention is as defined throughout the whole content of the present application.

In one aspect, the kit comprising a cell-free extracellular matrix produced by the methods described herein, and optionally, instructions for use thereof.

There is also provided a method of preparing a macromolecule solution for use in stem cell culture, comprising of preparing a solution comprising at a mixture of at least two macromolecules as defined throughout the whole content of the present application. In particular, the macromolecule(s) solution may have a viscosity of less than 2 mPa·s, more in particular, a viscosity of about 1 mPa·s.

### EXEMPLIFICATION

### Example 1

### Methods/Materials

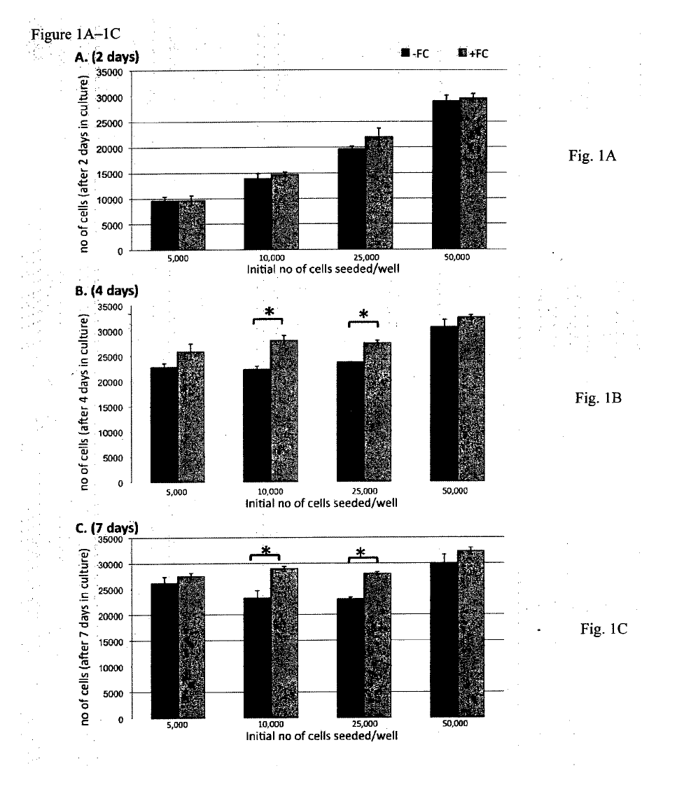

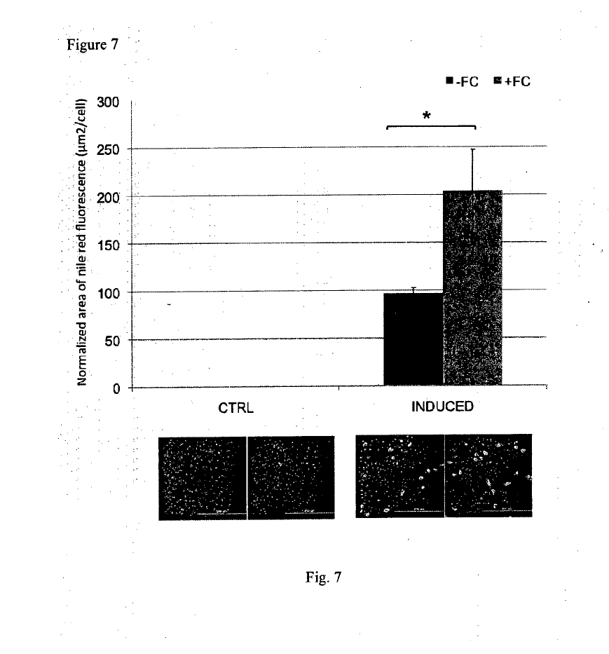

For FIGS. 1A-1C which illustrate the effect of macromolecular crowding on human mesenchymal stem cell proliferation in short term cultures at different seeding densities 5000, 10,000, 25000 and 50000 cells per well (1.8 cm²), hMSC (Cambrex) were seeded in control media (DMEM, 1000 mg/l glucose, 10% fetal calf serum (Gibco), penicillin/streptomycin, 2 mM L-Glutamine), then changed to control media or media containing ficoll cocktail (37.5 mg/ml FC70; 25 mg/ml FC400) the day after (day 0). Ficoll cocktail increased the proliferation of adult bone marrow human mesenchymal stem cells. Cell numbers were determined by adherent cytometry on day 2 (FIG. 1a), 4 (FIG. 1B) and 7 (FIG. 1C) using Metamorph® software and staining with DAPI (4′,6-diamidino-2-phenylindole). Every bar in the figures represents the average and standard deviation of triplicate sampling. Abbreviations: FC=ficoll cocktail.

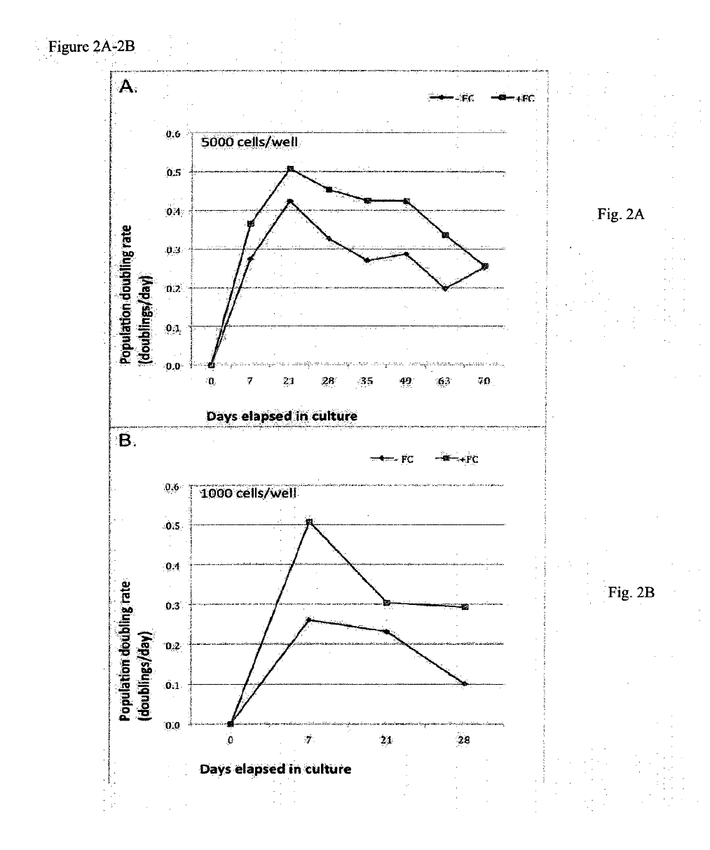

For FIGS. 2A-2B which illustrate the long-term effect of macromolecular crowding on the population doubling rate of human mesenchymal stem cell seeded at (FIG. 2A) 5000 cells per well and (FIG. 2B) 1000 cells per well (1.8 cm²), hMSC (Cambrex) were seeded in control media (DMEM, 1000 mg/l glucose, 10% fetal calf serum (Gibco), penicillin/streptomycin, 2 mM L-Glutamine), then changed to control media or media containing ficoll cocktail (37.5 mg/ml FC70; 25 mg/ml FC400) the day after (day 0). Cell numbers were determined every 7 days after seeding, for 70 and 28 days, respectively using Metamorph® software and staining with DAPI. Every point in the line represents the average of triplicate sampling.

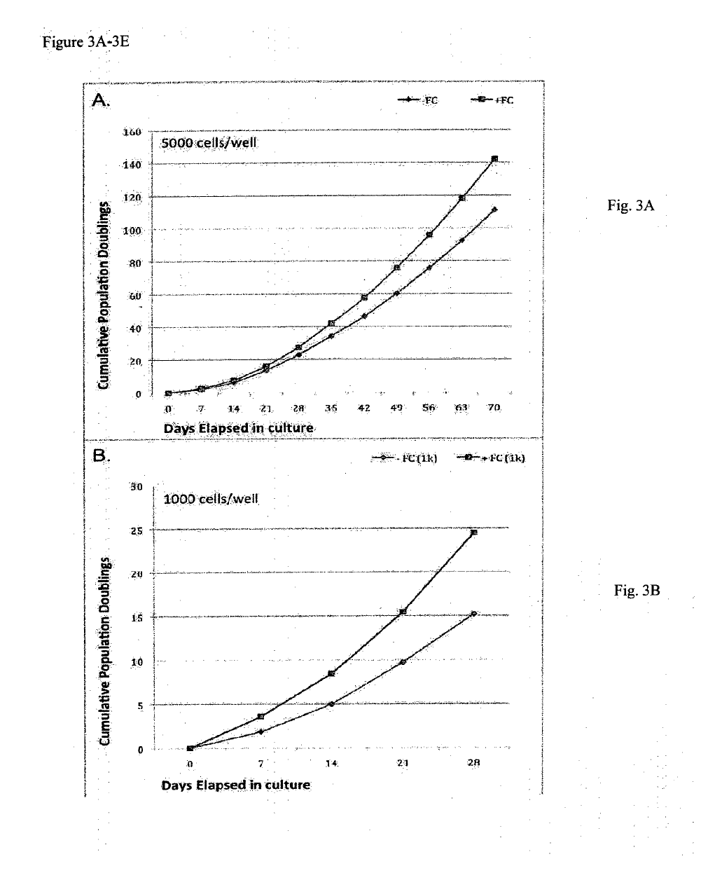

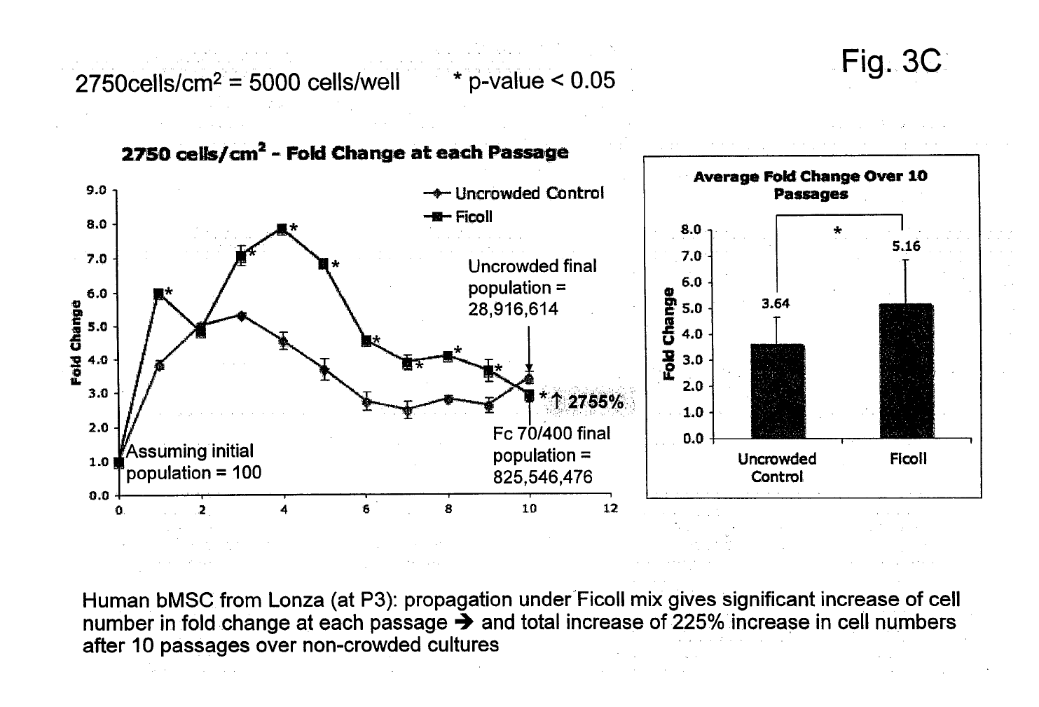

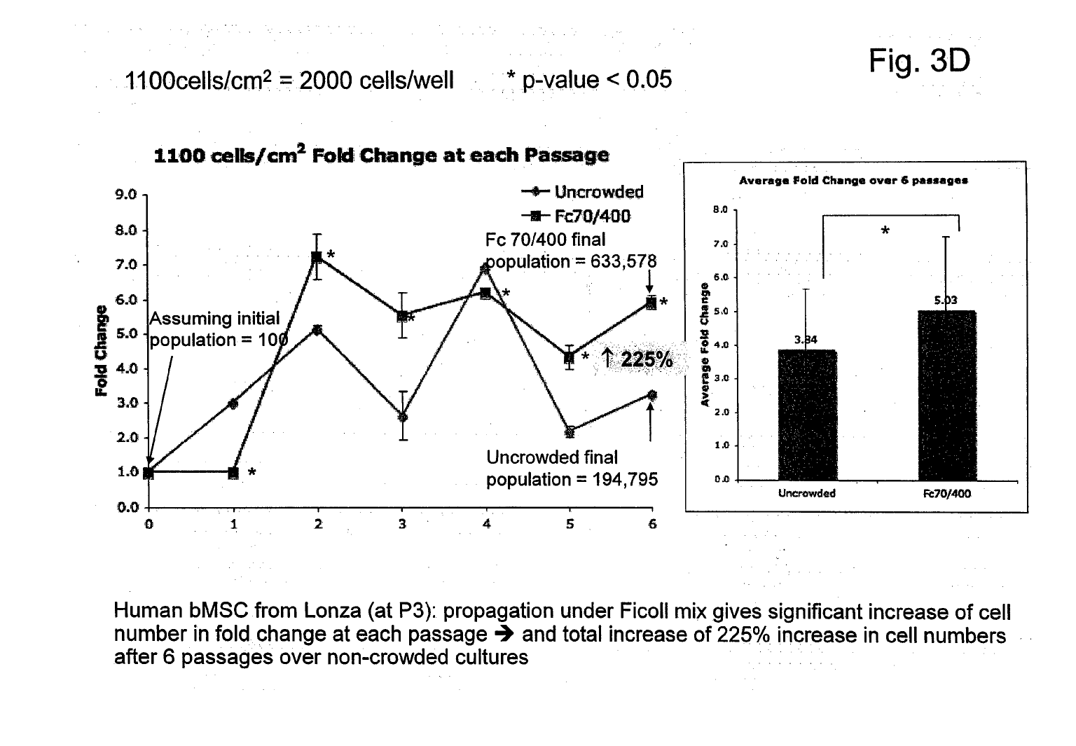

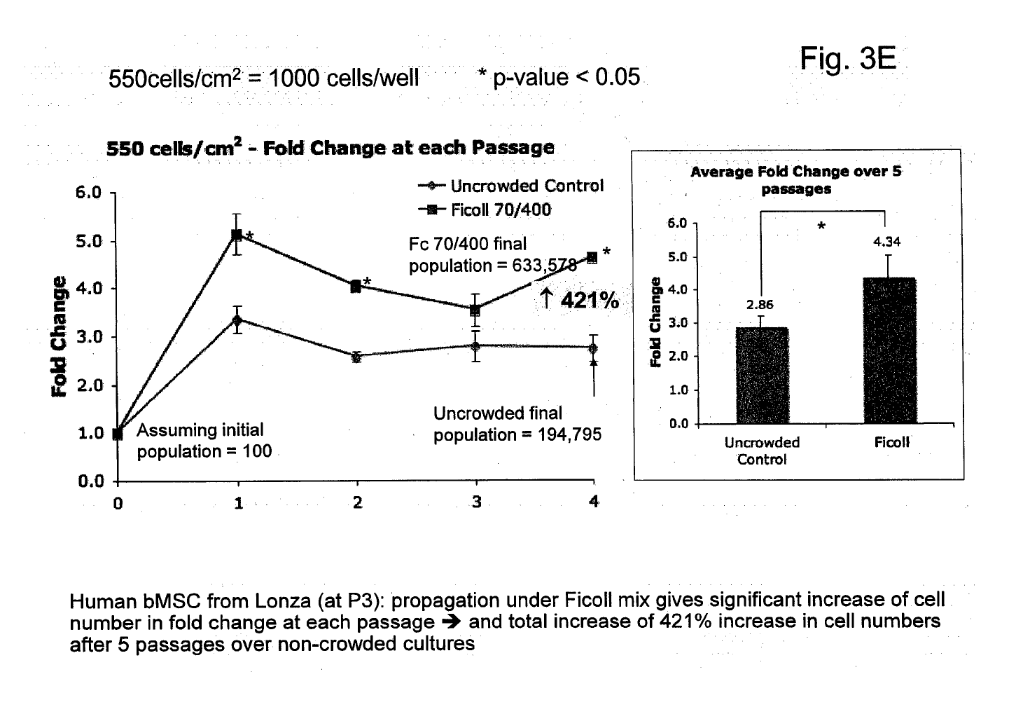

For FIGS. 3A-3B which illustrate the long-term effect of macromolecular crowding on the cumulative population doubling of human mesenchymal stem cell seeded at (FIG. 3A) 5000 cells per well and (FIG. 3B) 1000 cells per well (1.8 cm²), hMSC (Cambrex) were seeded in control media (DMEM, 1000 mg/l glucose, 10% fetal calf serum (Gibco). penicillin/streptomycin, 2 mM L-Glutamine), then changed to control media or media containing Ficoll cocktail (37.5 mg/ml FC70; 25 mg/ml FC400) the day after (day 0). Cell numbers were determined every 7 days after seeding, for 70 and 28 days days respectively using Metamorph® software and staining with DAPI. Every point in the line represents the average of triplicate sampling.

For FIGS. 3C-3E which illustrate the long-term effect of macromolecular crowding on the fold change increase in cell numbers of human mesenchymal stem cell seeded at (FIG. 3C) 5000 cells per well and (FIG. 3D) 1000 cells per well and (FIG. 3E) 2000 cells per well (well area=1.8 cm²). Fold change was calculated by dividing final cell number of each passage by initial seeding density.

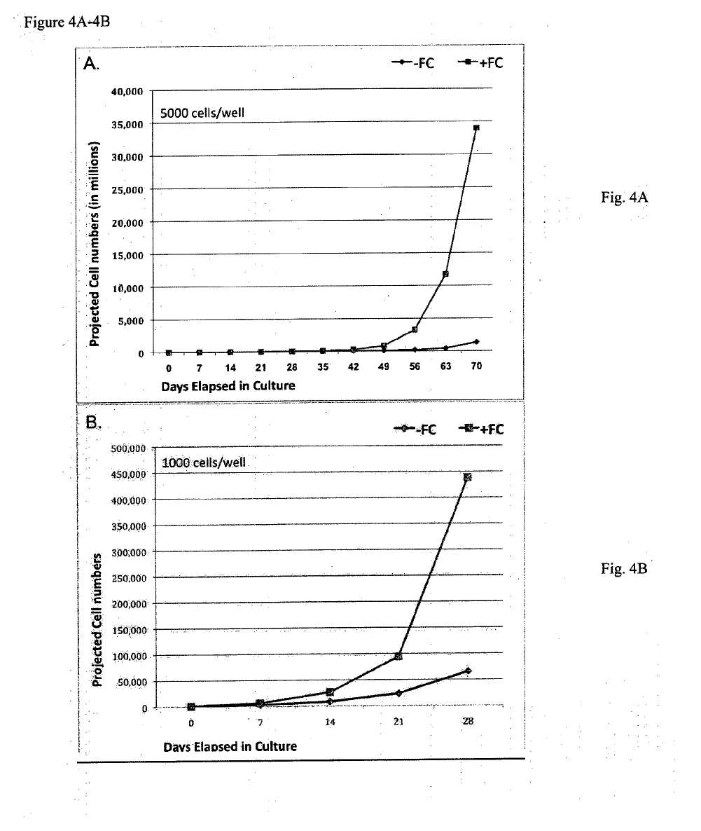

For FIGS. 4A-4B which illustrate the hypothetical cell numbers that would be obtained in the absence or presence of macromolecular crowding if they were propagated under the current density of (FIG. 4A) 5,000 cells per 1.8 cm² (2,800/cm²) well and (FIG. 4B) 1000 cells per 1.8 cm² (560/cm²) after 28 days and 70 days, respectively.

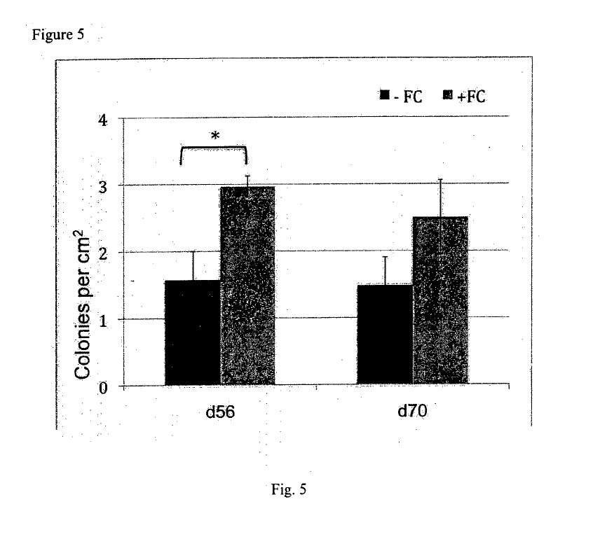

For FIG. 5 which illustrates the effect of macromolecular crowding on the colony forming unit (CFU) ability of the human mesenchymal stem cells after being cultivated in the absence (−FC) or presence of crowders (+FC) for 56 days (d56) and 70 days (d70), hMSC (Cambrex) were propagated in control media (DMEM, 1000 mg/l glucose, 10% fetal calf serum (Gibco), penicillin/streptomycin, 2 mM L-Glutamine), then changed to control media or media containing ficoll cocktail (37.5 mg/ml FC70; 25 mg/ml FC400) the day after (day 0). Cells were passaged every 7 days after seeding and were reseeded at 5000 cells per well (1.8 cm²) and changed to the respective medium the following day. At day 56 (d56) and day 70 (d70), some cells were seeded at 100 cells per 10 cm² in control medium and allowed to proliferate for 7 days. The wells were then methanol fixed and then stained with either crystal violet (0.5%) or DAPI and colonies bigger than 1 mm in diameter were counted visually under 10× magnification. Every bar represents the average of triplicate sampling.

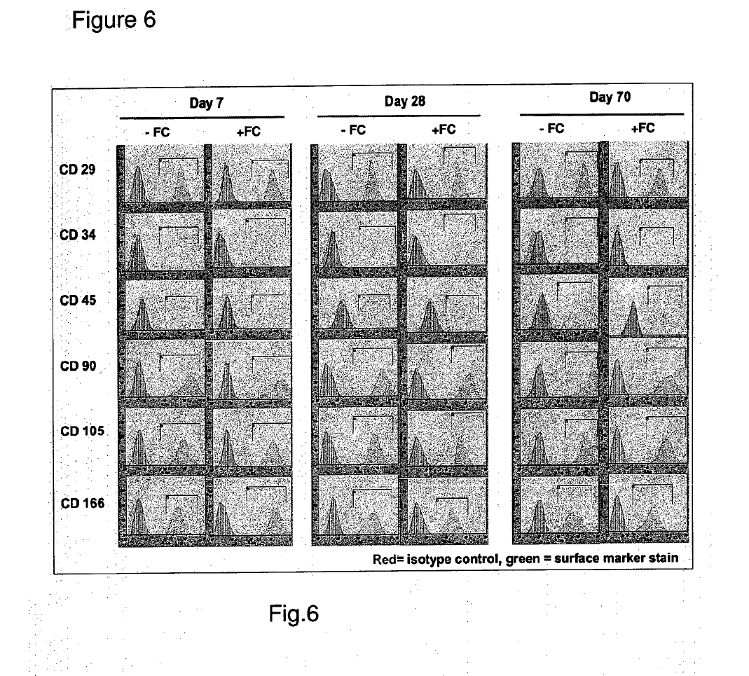

For FIG. 6 which illustrates the effect of macromolecular crowding on human mesenchymal stem cell surface marker expression, hMSCs (Cambrex) were plated at 3300 cells/cm² in T175 culture flask (celstar) and cultivated in the absence (−FC) or presence of crowders (+FC) for 7 days (d7), 28 days (d28) and 70 days (d70) before assessing cells for cell surface markers CD29, CD34, CD45, CD90, CD105, CD166 (all from Pharmingen) by antibody staining based flow cytometry. Each point represents the average and data of triplicate experiments.

For FIG. 7 which illustrates the effect of macromolecular crowding during pre-propagation of human mesenchymal stem cells and the subsequent effect on their adipogenic differentiation ability, hMSCs (Cambrex) were plated at 3300 cells/cm² in T175 culture flask (Celstar) and propagated in control media (DMEM, 1000 mg/l glucose, 10% fetal calf serum (Gibco). penicillin/streptomycin, 2 mM L-Glutamine), then changed to control media or media containing ficoll cocktail (37.5 mg/ml FC70; 25 mg/ml FC400) the day after (day 0). Cells were passaged every 7 days after seeding and reseeded at same density and change d to their respective medium the following day. At day 28 (d28), cells were trypsinised and seeded at 35000 cells per well (1.8 cm²) and induced to adipogenically differentiate as described previously (Pittenger, M F., et al., Science, 5411:143-147 (1999)) for 3 weeks. Monolayers were then fixed in 4% formaldehyde (30 min; RT) then stained with Nile red (Sigma-Aldrich; 0.05 mg/ml in water; 1 hr), a fluorescent lysochrome specific for cytoplasmic lipid droplets and co-stained with 4′,6-diamidino-2-phenylindole (DAPI; 0.5 μg/ml; 30 min). Adherent fluorescent cytometry was based on a montage of 9 sites per well taken by a coolSNAP HQ camera attached to a Nikon TE2000 microscope at 2× magnification, covering 83% of total well area. Nile red was viewed under a single rhodamine filter [Ex 572 nm/Em 630 nm] while DAPI fluorescence was accessed with a single Dapi filter [Ex 350 nm/Em 465 nm]. Measured nile red events were thresholded and lipid accumulation was measured by an image analysis software (MetaMorph 6.3v3). The extent of adipogenic differentiation was quantified by area of nile red fluorescence from thresholded events then normalized to nuclei count based on detected DAPI fluorescence. End data correspond to total area of lipid droplets present per well relative to cell number. The area measurements were imported into Microsoft Excel, and the mean±SD of the areas was calculated for control and crowded cells.

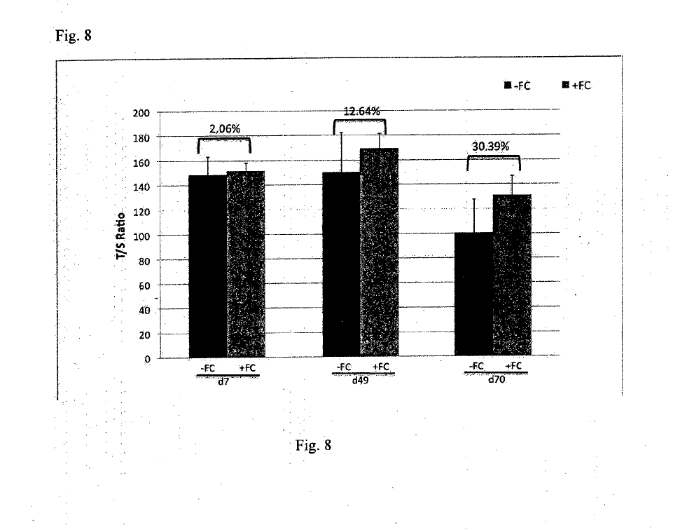

For FIG. 8 which illustrates the effect of macromolecular crowding on the relative telomere length of the human mesenchymal stem cells, hMSCs (Cambrex) were plated at 3300 cells/cm² in T175 culture flask (CellStar) and propagated in control media (DMEM, 1000 mg/l glucose, 10% fetal calf serum (Gibco) penicillin/streptomycin, 2 mM L-Glutamine), then changed to control media or media containing ficoll cocktail (37.5 mg/ml FC70; 25 mg/ml FC400) the day after (day 0). Cells were passaged every 7 days after seeding and reseeded at same density and changed to their respective medium the following day. Following the method described by Cawthon R M, Nucleic Acids Res, 10:e47 (2008)), relative telomere lengths was determined by quantitative PCR by measuring the factor by which each DNA sample differed from a reference DNA sample in its ratio of telomere repeat copy number to single copy gene copy number (T/S ratio). The T/S ratio is approximately [2^{Ct(telomeres)}/2^{Ct(36B4)}]⁻¹=2^{-Δct}, the factor difference in T/S ratio between test and control samples gives rise to relative difference in telomere length. At day 7 (d7), day 49 (d49) and day 70 (d70), total genomic DNA was extracted using the DNeasy Blood & Tissue Kit (Qiagen) following the manufacturer's protocol. Real time polymerase chain reactions was performed and monitored on a Stratagene real-time PCR instrument (Stratagene) with a PCR master mix based on Platinum Taq DNA polymerase (fermentas). Data analysis was performed using the MxPro software (Stratagene). For each sample, the Ct value was defined as the cycle number at which the fluorescence intensity reached the amplification based-threshold fixed by the instrument-software.

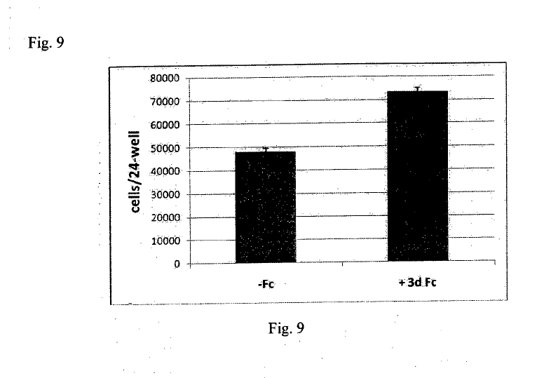

For FIG. 9 which illustrates the effect of macromolecular crowding on the cell numbers of fibrocytes after seven days in 24-well plate wells, fibrocytes were cultured under conditions described by Kuwana, M, et al., J Leukoc Biol, 74(5):833-845 ((2003)). Shortly, isolated peripheral blood mononuclear cells were seeded onto fibronectin-coated dishes at a cell density of 1 million cells per well in low glucose DMEM supplemented with 10% FBS under crowded conditions (Ficoll-cocktail) for seven days. Cells were grown in crude culture for three days, after which the medium was renewed and non-adherent cells were removed. The remaining cells were grown for 4 further days. Cells were grown either under uncrowded conditions (−Fc) or in the presence of Ficoll-cocktail during the first 3 days (+3d Fc). On day seven adherent cells were washed with twice with PBS and fixed with ice-cold methanol. Wells were blocked with 3% BSA in PBS and incubated with Dapi for 30 minutes. After incubation wells were washed thrice with PBS and stored in 500 μl PBS. Dapi stained nuclei were counted by adherent cytometry and represent the cell number per 24-well plate well.

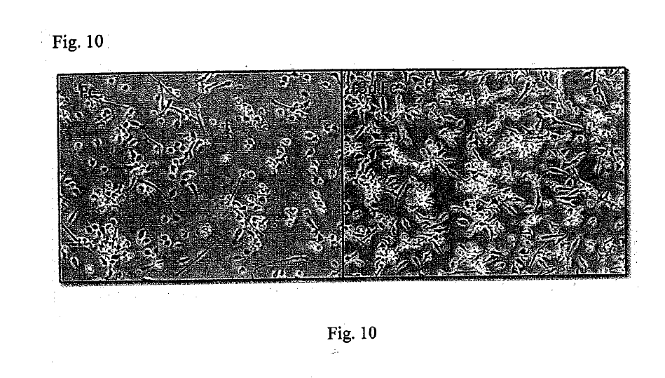

For FIG. 10 which shows phase contrast photographs (10× magnification) of fibrocytes growing on fibronectin-coated dishes for seven days (Kuwana, M, et al., J Leukoc Biol, 74(5):833-845 ((2003)), isolated peripheral blood mononuclear cells were seeded onto serum-protein coated dishes in low glucose DMEM supplemented with 10% FBS in the absence of crowders (−Fc) or with the addition of Ficoll-cocktail during the first 3 days (+3d Fc). Non-adherent cells were removed during the media change on day three. After seven days adherent spindle-shaped cells were present in culture. These cells represent fibrocytes.

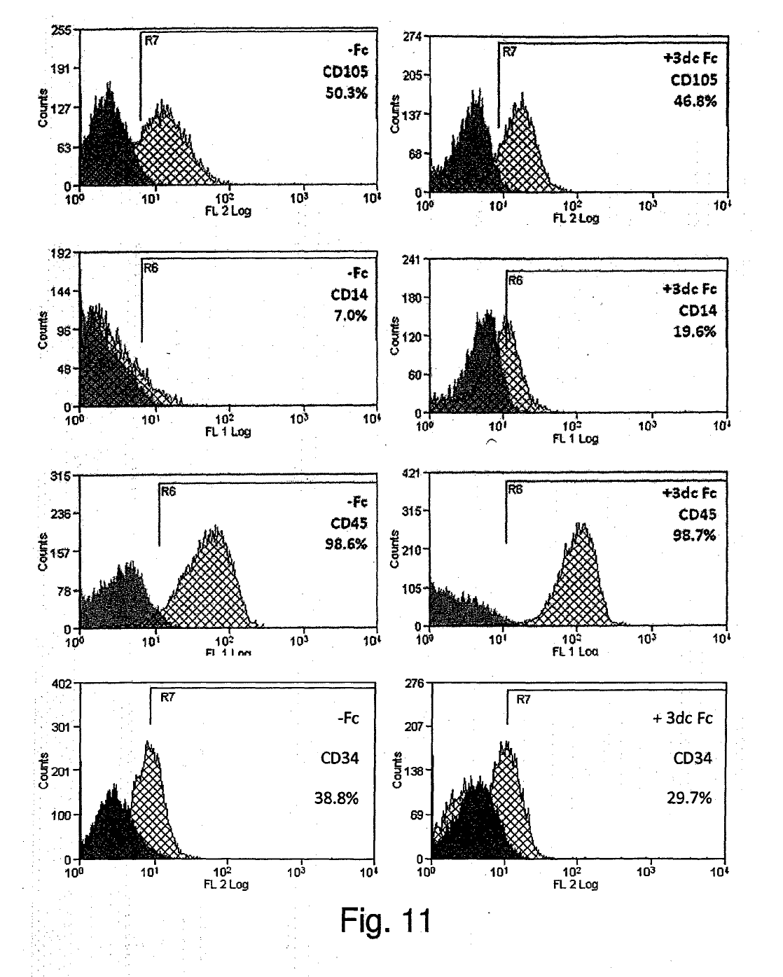

For FIG. 11 which shows flow cytometric analysis of the adherent partion/fraction of peripheral blood mononuclear cells after 1 week of culture, isolated peripheral blood mononuclear cells were seeded onto fibronectin-coated dishes in low glucose DMEM supplemented with 10% FBS without the addition of crowders (−Fc) or in the presence of Ficoll-cocktail for the first 3 days (+3d Fc). Non-adherent cells were removed during the media change on day three. Adherent cells were removed from the culture dishes, resuspended in PBS buffer supplemented with 0.5% FBS and incubated with FITC- or PE-conjugated antibodies (BD sciences) for 30 min. Cells were washed once with PBS buffer supplemented with 0.5% FBS and fixed in 1% formladehyde in PBS. Samples were analysed with the Cyan flow cytometer (Dako Cytomation). Cells were tested for stem cell marker CD105, monocyte marker CD14 and leukocyte marker CD45, and hematopoetic stem cell marker CD34.

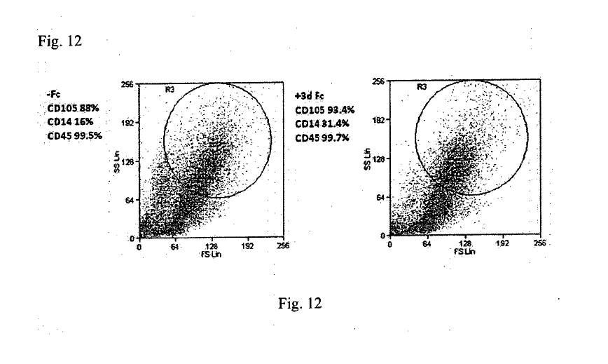

For FIG. 12 which shows the flow cytometric analysis of the adherent partion/fraction of peripheral blood mononuclear cells after 1 week of culture, isolated peripheral blood mononuclear cells were seeded onto fibronectin-coated dishes in low glucose DMEM supplemented with 10% FBS without the addition of crowders (−Fc) or in the presence of Ficoll-cocktail for the first 3 days (+3d Fc). Non-adherent cells were removed during the media change on day three. Adherent cells were removed from the culture dishes, resuspended in PBS buffer supplemented with 0.5% FBS and incubated with FITC- or PE-conjugated antibodies (BD sciences) for 30 min. Cells were washed once with PBS buffer supplemented with 0.5% FBS and fixed in 1% formladehyde in PBS. Samples were analysed with the Cyan flow cytometer (Dako Cytomation). Cells were tested for CD105, CD14 and CD45. Populations with the highest percentage of cells positive for fibrocyte markers were gated.

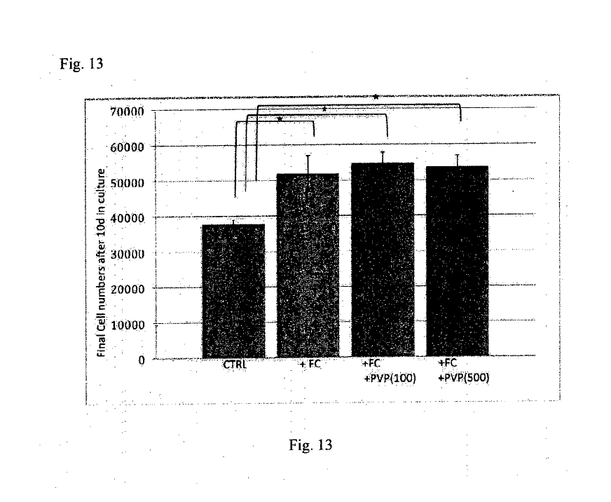

For FIG. 13 which illustrates the effect of a ternary combination of macromolecular crowders (Ficoll and PVP) on human mesenchymal stem cell proliferation in culture for 10 days, hMSC (Cambrex) were seeded at 10,000 cells per well in control media (DMEM, 1000 mg/l glucose, 10% fetal calf serum (Gibco), penicillin/streptomycin, 2 mM L-Glutamine), then changed to control media or media containing ficoll cocktail (37.5 mg/ml FC70; 25 mg/ml FC400) or media containing ficoll cocktail (37.5 mg/ml FC70; 25 mg/ml FC400) spiked with polyvinylpyrrolidone (100 μg/ml) or media containing ficoll cocktail (37.5 mg/ml FC70; 25 mg/ml FC400) spiked with polyvinylpyrrolidone (5004 ml) (purple bars); the day after (day 0). Ficoll cocktail increases the proliferation of adult bone marrow human mesenchymal stem cells. Cell numbers were determined by adherent cytometry using Metamorph® software and staining with DAPI (4′,6-diamidino-2-phenylindole). Every bar in the figures represents the average and standard deviation of triplicate sampling. Abbreviations: FC=ficoll cocktail, PVP=polyvinylpyrrolidone.

### Results

Macromolecular Crowding with Ficoll cocktail is effective in increasing the proliferation of adult bone marrow human mesenchymal stem cells at different seeding densities ranging from 5000, 10,000, 25,000 and 50,000 cells per well (1.8 cm²) or (2800, 5600, 13900 and 27,800 cells/cm², respectively) compared to control after 2, 4 and 7 days in culture (FIGS. 1A-1C). Although it was observed that after 7 days—all wells reach confluence at ˜35,000 cells/well regardless of initial seeding or the presence of crowding due to surface area limitations, this indicates that it is likely more effective to use macromolecular crowding with a lower seeding density or larger surface area.

The overall increase in proliferation of the hMSCs cultured in the presence of macromolecular crowding indicates that for a given period of time the hMSCs undergo more population doublings (PD). Since PD is limited in these cells, it was expected that the cells would reach replicative senescence earlier if kept in culture for a long term. Surprisingly, it was demonstrated that cell proliferation slowed down after a similar time in culture as the control to ˜0.25PD/day in 70 days (FIG. 2A), but yielded 30% more cumulative population doubling after 70 days in culture in that time window (FIG. 3A).

The actual population doubling data at a seeding density of 5000 cells/well indicate that this method allows one to obtain 3.4×10¹⁰ cells with macromolecular crowding compared to 1.3×10⁹ with current culturing conditions after 70 days in culture (FIG. 4A). At a seeding density of 1000 cells/well, it is likely one can obtain 4.4×10³ cells with macromolecular crowding compared to 6.5×10² with current culturing conditions after 28 days in culture (FIG. 4B). Under both conditions, a magnitude more cells under macromolecular crowding compared to control conditions was obtained, indicating that the methods provided herein can be used for generating more cells for translational purposes.

The hallmark of the most “stem-like” cells in the heterogeneous hMSC population is their ability to form colonies when seeded at low density thus providing a phenotype to confirm the effect of macromolecular crowding. Therefore, a colony forming assay in control media was set up and the results showed that hMSCs that were pre-propagated under macromolecular crowding for 56 days and 70 days were able to form 2 times and 1.7 times as many colonies as the control, respectively (FIG. 5).

The results obtained with surface markers of the stem cells, indicate that the human mesenchymal stem cells cultured in the presence of macromolecular crowding are true mesenchymal stem cells exhibiting the following surface characteristics, CD29+, CD34−, CD45−, CD90+, CD105+ and CD166+ even after culturing in the presence of macromolecular crowding for 7, 49 and 70 days (FIG. 6).

Interestingly, it was observed that after 28 days in culture in the presence of macromolecular crowding, hMSCs were 2× more responsive to adipogenic induction than those cultured over the same time period in the absence of crowding, indicating that macromolecular crowding may have a pre-priming effect. Adipogenic induction was carried out in DMEM with 10% FCS, 10 μg/ml insulin, 0.5 mM methlisobutylxanthine, 1 flM Dexamethasone), proving that macromolecular crowding not only increases hMSC proliferation but also preserves their “stemness”, thus their capability to differentiate is not impaired (FIG. 7).

Using a novel method of determining relative telomere lengths as described by Cawthon R M, et al., Nucleic Acids res, 10:e47 (2002) it was determined that hMSCs cultured under macromolecular crowding for 7 days, 49 days and 70 days had telomeres that were 2.06%, 12.64% and 30.39% longer that those in control medium respectively (FIG. 8). This is a surprising finding as the stem cells in the presence of a crowder cocktail had undergone more population doublings then the controls and with a similar time window of a slowing proliferation rate. This demonstrates that despite a faster population doubling rate senescence is not only kept at bay but slowed done in comparison to non-croweded MSC's.

For fibrocytes, the highest cell numbers are yielded when cells were cultured under crowded conditions. An increase of 1.5 fold in cell number is induced by the Fc-cocktail (FIG. 9). As the percentage of cells expressing fibrocyte markers are about the same under crowded and non-crowded conditions, whereby fibrocytes markers are coexpressed (FIGS. 10, 11 and 12), there is a huge increase in fibrocytes number after 1 week when cultured under crowded conditions.

A tertiary cocktail comprising addition of PVP at 100 μg/ml to the original ficoll cocktail (37.5 mg/ml FC70; 25 mg/ml FC400) results in better proliferation compared to just the ficoll cocktail alone and 48% better than control conditions.

### Example 2

### Invention Disclosure

### Material & Methods

### Mesenchymal Stem Cell Culture

Human bone-marrow derived mesenchymal stem cells (MSCs) were obtained commercially (Cambrex Bio. Science Walkersville) at passage 2 (p2) and cultured in a basic culture medium composed of low glucose Dulbecco's modified Eagle's medium (LG DMEM, Gibco-BRL) supplemented with Glutamax, 10% fetal bovine serum, 100 units/ml penicillin and 100 μl/ml streptomycin. Cells were maintained at 37° C. in a humidified atmosphere of 5% CO₂/95% air, with medium change twice per week. To prevent spontaneous differentiation, cells were maintained at subconfluent levels prior to being detached using 0.5% trypsin/1 mM EDTA, passaged at 1:4 and cultured to generate subsequent passages. Directed differentiation was carried out with cells at passage 4 (p4) and 8 (p8).

### Adipogenic Induction of Mesenchymal Stem Cells (MSC)

### Induction

MSCs at an early passage (p4) and late passage (p8) were seeded at an initial density of 5×10⁴ cells/well in 24-well plates (Celstar). Adipogenic differentiation was stimulated 48 hours post-seeding as described (Pittenger, M F et al., Science, 5411:143-147 (1999)) via three cycles of induction/maintenance, feeding with induction medium for 4 days (37° C., 5% CO₂) followed by 3 days of culture in basal medium. Non-induced control MSC were fed only basal medium on the same schedule. Basal medium was composed of high glucose Dulbecco's modified Eagle's medium (HG DMEM, Gibco-BRL) supplemented with 10% fetal bovine serum, 100 units/ml penicillin, 1000/ml streptomycin and insulin (0.01 mg/mL) while induction medium was supplemented with 3-Isobutyl-1-methylxanthine (0.5 mM), dexamethasone (10-7M) and indomethacine (0.2 mM). Crowding is effected by the addition of a crowding cocktail during both the induction and maintenance phase. Induction cocktail was diluted at the following ratios—1:0 (100%), 1:4 (20%) and 0:1(0%) to assess the potential of crowding to cause a more effective use of less inductive ingredients. EVE was created by supplementation of media with a cocktail of macromolecules Ficoll™ 70 (Fc70; Sigma-Aldrich, St Louis) at 37.5 mg/ml and Ficoll™ 400 (Fc400; Sigma-Aldrich, St Louis) at 25 mg/ml.

### Nile Red Adherent Cytometry to Assess Area of Cytoplasmic Lipid Accumulation

At three time points (7, 14, 21 days) corresponding to the end of each induction cycle, monolayers in 24-well plates were rinsed with PBS, fixed in 4% formaldehyde (30 min; RT) then stained with Nile red (Sigma-Aldrich; 0.05 mg/ml in water; 1 hr), a fluorescent lysochrome specific for cytoplasmic lipid droplets and co-stained with 4′,6-diamidino-2-phenylindole (DAPI; 0.5 μg/ml; 30 min). Adherent fluorescent cytometry was based on a montage of 9 sites per well taken by a coolSNAP HQ camera attached to a Nikon TE2000 microscope at 2× magnification, covering 83% of total well area. Nile red was viewed under a single rhodamine filter [Ex 572 nm/Em 630 nm] while DAPI fluorescence was accessed with a single Dapi filter [Ex 350 nm/Em 465 nm]. Measured nile red events were thresholded and lipid accumulations were measured by an image analysis software (MetaMorph 6.3v3). The extent of adipogenic differentiation was quantified by area of nile red fluorescence from thresholded events then normalized to nuclei count based on detected DAPI fluorescence: End data corresponded to total area of lipid droplets present per well relative to cell number. The area measurements were imported into Microsoft Excel, and the mean±SD of the areas was calculated for control and crowded cells.

Nile Red Flow Activated Cytometry to Assess Percentage Differentiated

Monolayers were washed twice in PBS, trypsinized and pelleted by centrifugation for 5 min at 2000 rpm at room temperature. Resuspended pellets were then stained with Nile red (0.05 mg/ml in water; 1 hr) then fixed by adding PFA to a concentration of 0.5% as described previously (Gimble et al). The samples were then analyzed with a FACScan™ flow cytometer (Becton Dickinson, Basel, Switzerland). Nile Red fluorescence was measured on the FL2 emission channel through a 585±21 nm band pass filter, following excitation with an argon ion laser source at 488 nm. Using a forward scatter (FSC)/side scatter representation of events, a gating region was defined to exclude cellular debris from the analysis. The upper right (UR) quadrant was then established on the FL2/FSC blot of the gated population in order to count cells with high FL2 values (adipocytes). Data analysis was performed using CellQuest1 3.1 software (Becton Dickinson). For each sample 10,000 events were collected. The results are expressed as the percentage of cells appearing in the upper right quadrant (UR).

### Gene Expression of Fatty Acid Binding Protein-4 and Leptin

Total RNA was extracted using the RNAeasy single step column spin following the manufacturer's protocol. cDNA will be synthesized from isolated mRNA using Superscript reverse transcriptase II. RT-PCR reactions will be performed and monitored on a Stratagene real-time PCR instrument (Stratagene) with a PCR master mix based on Platinum Taq DNA polymerase (Invitrogen). Data analysis was performed using the MxPro software (Stratagene). For each cDNA sample, the Ct value was defined as the cycle number at which the fluorescence intensity reached the amplification based-threshold fixed by the instrument-software. Relative expression level for fatty acid binding protein-4 (aP2/FABP4) and leptin (Lp) was calculated by normalizing the quantified cDNA transcript level (Ct) to the housekeeping gene, RLPO.

### Osteogenic Induction of Mesenchymal Stem Cells

### Induction

MSCs at an early passage (p4) and late passage (p6) were plated in 24-well plates (Celstar) at an initial density of 7.5×10³ cells/well in basal medium composed of high glucose Dulbecco's modified Eagle's medium (HG DMEM, Gibco-BRL) supplemented with 10% fetal bovine serum, 100 units/ml penicillin, 100 μl/ml streptomycin and insulin (0.01 mg/mL). Osteogenesis was stimulated was stimulated 24 hours post-seeding as described previously (Jaiswal, N, et l., J Cell Biochem, 64(2):295-312 (1997)) with basal media supplemented with ascorbate-2-phosphate (10 uM), b-glycerophosphate (10 mM), 1,25-dihydroxy Vitamin D (0.01 uM) and dexamethasone (10-7M). Media changes were performed twice weekly. Non-induced control hMSC were fed only basal media on the same schedule. Induction cocktail was diluted to the following concentrations—100%, 20% and 0% to assess the potential of crowding to cause a more effective use of less inductive ingredients. EVE was created by supplementation of media with a cocktail of macromolecules Ficoll™ 70 (Fc70; Sigma-Aldrich, St Louis) at 37.5 mg/ml and Ficoll™ 400 (Fc400; Sigma-Aldrich, St Louis) at 25 mg/ml. See FIG. 47.

### Alizarin Red Staining for Calcium

Monolayers in 24-well plates were washed with PBS, fixed in 4% methano-free formaldehyde solution (10 min; RT), then washed twice with PBS and incubated with 40 mM ARS (20 min; RT) with gentle shaking. Unincorporated dye was removed and wells were washed three times in water and air-dried, then visualized by bright field microscopy.

### Results

### Macromolecular Crowding Enhances Adipogenesis by Increasing the Amount of Cytoplasmic Lipid Droplet Accumulation

Induced differentiation of hMSCs toward adipogenesis resulted in a larger cell morphology and a time-dependent increase in intracellular lipid-filled droplets stained by nile red. To analyze the effect of EVE on the amount of lipid accumulation during adipogenic induction, hMSCs at passage 4 and 8 were seeded into celstar 24-well plates and cultured for 21 days as described under materials and methods. Subsequently, plates at days 7 and 14 days were fixed, co-stained with nile red and DAPI then subjected to adherent fluorescent cytometry. Results depicted in FIGS. 14A and 14B, showed that by day 7, cultures of hMSCs at p4 and 8 exhibited higher accumulation of lipid droplets in the presence of EVE at both full-strength and half-strength induction. Quantifying the area of nile red stained lipid droplets by adherent fluorescent cytometry, it was found that EVE caused a fold increase of 2.7±0.005 and 2.6±0.006 for passage 4 at half-strength and full strength respectively (FIG. 14B). A similar trend of 2.2±0.04 and 2.6±0.06 fold increase at half-strength and full strength was observed for hMSCs at passage 8 (FIG. 14D).

### Crowding Increases the Proportion of MSCs Undergoing Adipogenesis

It was next determined whether crowding was not only able to increase the amount of lipid accumulated within the cytoplasm of differentiated hMSCs but also increase the proportion of MSCs that exhibit these droplets, as there is low efficiency in differentiation especially with cells obtained from older donors (Minguell J J, et al., Bone 6:919-926 (2003)). Flow cytometric data observed at end of 21 days (FIGS. 15A-15D) showed that treatment of confluent cultures with induction media in the absence of crowding resulted in 20.96% and 22.4% of the population becoming adipocytes under half- and full-strength induction respectively. In the presence of crowding, this was shifted up to 37.03% and 45.14%, corresponding to an increase of 1.8 fold and 2 fold under half- and full-strength induction respectively.

### Crowding Increased the Expression of Key Adipogenic Genes, PPARg AND AP2.

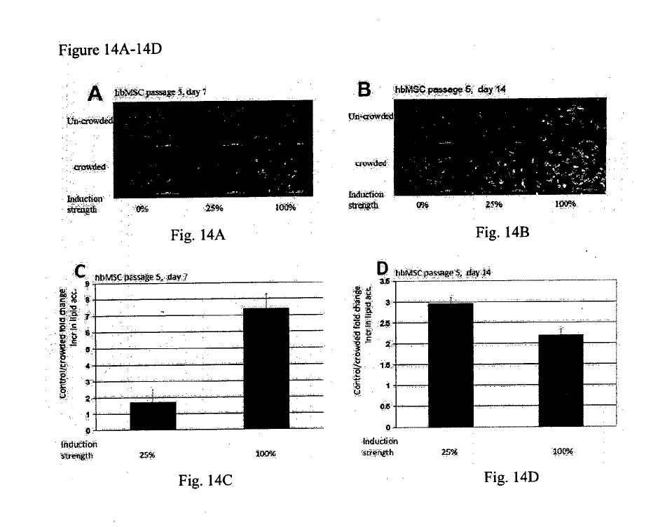

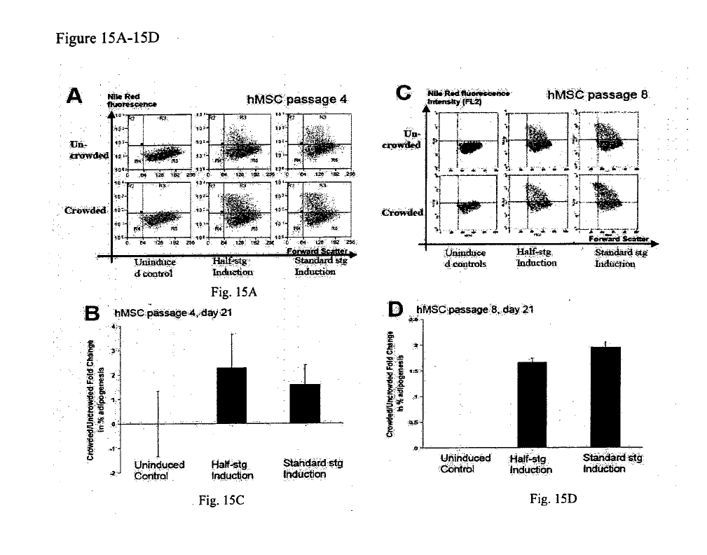

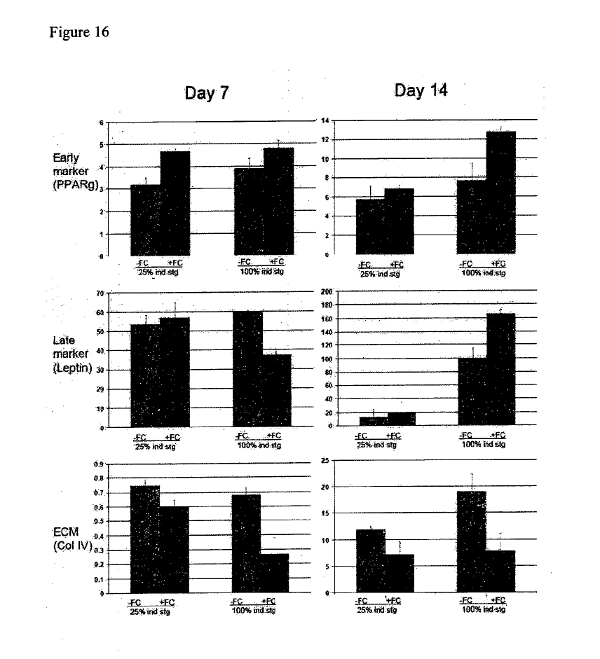

FIG. 16 shows the effects of macromolecular crowding on adipogenic gene expression. Crowding enhanced the expression of key adipogenic genes PPARg and Leptin. Interestingly, a decrease in collagen IV gene expression was observed although this was accompanied by an increase in actual protein deposited.

Crowding Increased the Deposition of Collagen IV in the Cell Layer (Western Blot), this is Accompanied by Decreased Expression of COL4 MRNA

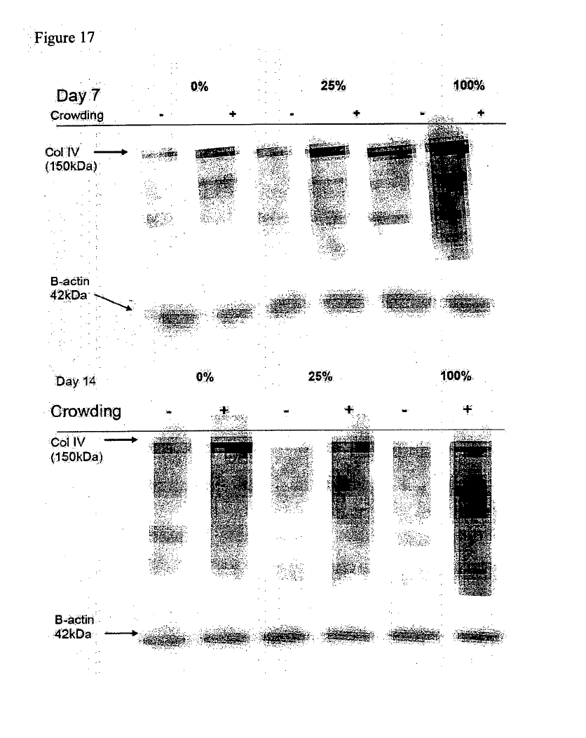

FIG. 17 shows the effects of macromolecular crowding on the amount of collagen N deposited. Results of western blot analysis of hBMSCs cultures at passage 5 after adipogenic induction at 25% or 100% in the absence or presence of crowding after 7 or 14 days. An increase in the collagen N band with crowding at both induction strength with the addition of crowding was observed.

### Effects of Macromolecular Crowding on the Amount of Calcification in Monoloayer

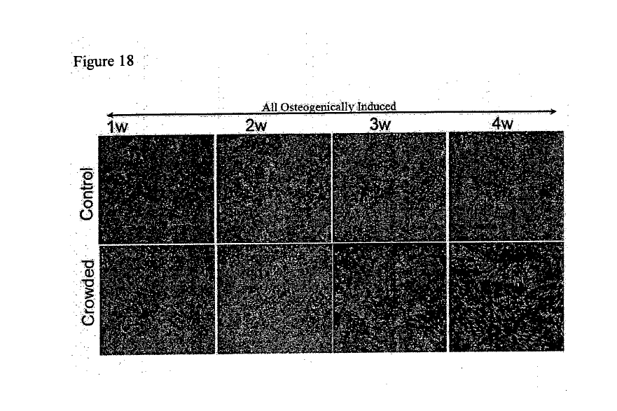

FIG. 18 shows the effects of macromolecular crowding on the amount of calcification in monolayer. Alizarin red staining of osteogenically induced monolayers in the absence or presence of crowding over 4 weeks indicates that crowding dramatically enhanced the amount of calcification in monolayers at week 3 and week 4, and thus, the extent of differentiation.

### CTB-FITC Staining of Rafts

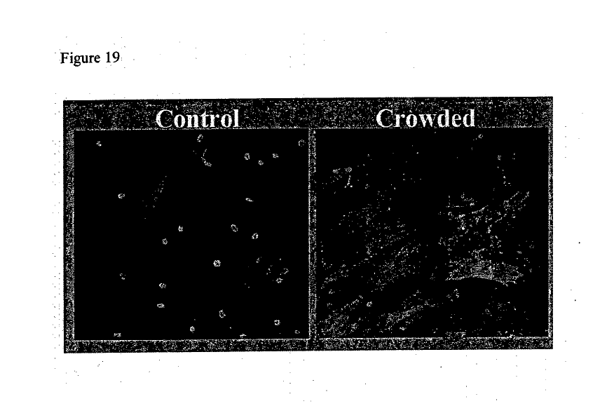

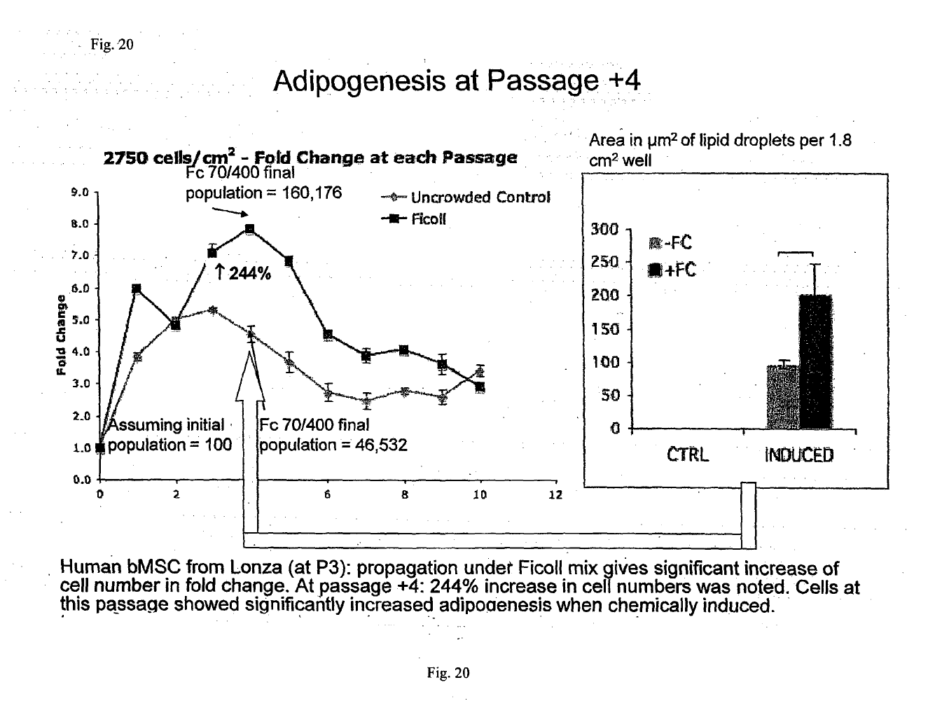

FIG. 19 shows the effects of macromolecular crowding on the expression level of lipid rafts on monolayer. Cholera Toxin B-FITC staining of monolayers cultured for 7 days in the absence or presence of crowding indicates that crowding dramatically enhances the amount of lipid rafts expressed in the monolayer.

### Mixed Macromolecular Crowding Increased Proliferation Rate of MSC on Cell Culture Plastic

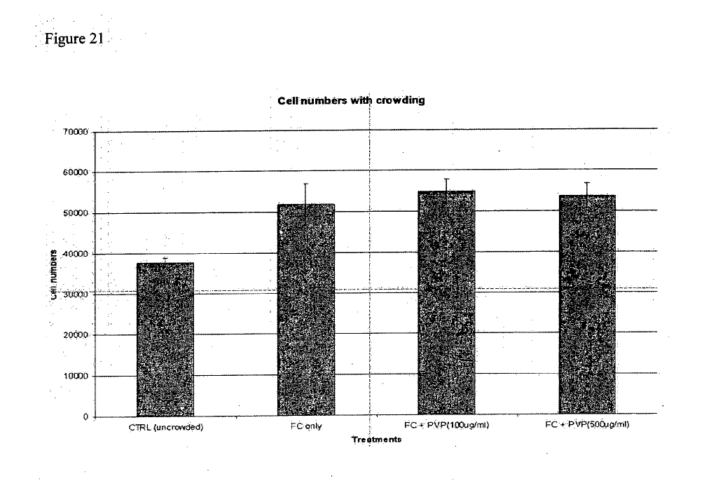

See FIGS. 21 and 3A-3C.

### Retained Differentation Potential at Increased Proliferation Rate Under Mixed Macromolecular Crowding

hMSCs (Cambrex) were plated at 3300 cells/cm2 in T175 culture flask (celstar) and propagated in control media (DMEM, 1000 mg/l glucose, 10% fetal calf serum (Gibco), penicillin/streptomycin, 2 mM L-Glutamine), then changed to control media (FIG. 7) or media containing ficoll cocktail (37.5 mg/ml FC70; 25 mg/ml FC400) (FIG. 7) the day after (day 0). Cells were passaged every 7 days after seeding and reseeded at same density and change d to their respective medium the following day. At day 28 (d28), cells were trypsinised and seeded at 35000 cells per well (1.8 cm²) and induced to adipogenically differentiate as described previously (Pittenger M F, et al., Science 5411:143-147) for 3 weeks. Monolayers were then fixed in 4% formaldehyde (30 min; RT) then stained with Nile red (Sigma-Aldrich; 0.05 mg/ml in water; 1 hr), a fluorescent lysochrome specific for cytoplasmic lipid droplets and co-stained with 4′,6-diamidino-2-phenylindole (DAPI; 0.5 μg/ml; 30 min) as described previously [Greenspan P, et al., J Cell Biol (3):965-973.]. Adherent fluorescent cytometry was based on a montage of 9 sites per well taken by a coolSNAP HQ camera attached to a Nikon TE2000 microscope at 2× magnification, covering 83% of total well area. Nile red was viewed under a single rhodamine filter [Ex 572 nm/Em 630 nm] while DAPI fluorescence was accessed with a single Dapi filter [Ex 350 nm/Em 465 nm]. Measured nile red events were thresholded and lipid accumulations were measured by an image analysis software (MetaMorph 6.3v3). The extent of adipogenic differentiation was quantified by area of nile red fluorescence from thresholded events then normalized to nuclei count based on detected DAPI fluorescence. End data correspond to total area of lipid droplets present per well relative to cell number. The area measurements were imported into Microsoft Excel, and the mean±SD of the areas was calculated for control and crowded cells.

Interestingly, it was observed that after 28 days in culture in the presence of macromolecular crowding, hMSCs were 2× more responsive to adipogenic induction that those cultured over the same time period in the absence of crowding, indicating that macromolecular crowding may have a pre-priming effect. Adipogenic induction was carried out in DMEM with 10% FCS, 10 ug/ml insulin, 0.5 mM methlisobutylxanthine, 1 flM Dexamethasone), proving that macromolecular crowding not only increases hMSC proliferation but also preserves their “stemness”, their capability to differentiate is not impaired (FIG. 7).

### Discussion

The attenuation of human MSC differentiation potentials following extensive ex vivo expansion is a major hurdle in their utilization during MSC-based tissue engineering applications. In this present study, the ability of macromolecular crowding was investigated via application of a neutral polyanionic cocktail to augment the adipogenic & osteogenic differentiation on hBMSCs at passage 5. Evaluation of several key markers associated with human MSC adipogenic differentiation such as expression leptin (Rosen, et al., Annu Rev Cell Dev Biol, 145-171 (2000)) and PPAR-gamma (Gregoire, F M, et al., Physiol Rev, 3:783-809 (1998)), and the ability to accumulate lipids (Azain, et al., Annu Rev Cell Dev Biol, 145-171 (2004)) were analyzed to quantify the extent and efficiency of differentiation. It was hypothesized that this was due to EVE speeding up the numerous enzymatic processes involved in matrix remodeling that occurs during differentiation thus generating a more favourable microenvironment that resembles the matured niche.

Demonstrated herein is that EVE imposed in culture enhanced the adipogenic differentiation of MSCs at p5, both in amount of lipid accumulated and the percentage of cells undergoing differentiation. Interestingly, more lipid droplet accumulation was observed at half-strength induction with EVE at both passages compared full strength induction in the absence of EVE, demonstrating that EVE enables more effective use of less active ingredients. In the controls, Ficoll did not cause adipogenesis, indicating that it does not have adipogenic inductive capability.

In conclusion, evidence that crowding augments the adipogenic induction of hbMSCs by augmenting the deposition and remodeling of the ECM, thus generating a more condusive microenvironment, is presented. Since MSCs represent a potential cell source for clinical repair of adipose tissue defects, the ability to enhance adipogenic differentiation of MSCs by increasing the amount of lipid accumulation and proportion of MSCs that undergo differentiation likely leads to the production of functional tissue equivalents in vivo. This technology also likely assists in augmenting the differentiation of late passage MSCs for tissue engineering purposes and reduce costs by decreasing the amount of active ingredients needed.

### Example 3

### Mixed Macromolecular Crowding is a Biophysical Driver of Mesenchymal Stem Cell Microenvironment Formation and Amplifies Adipogenic Differentiation Through Cell-Matrix Reciprocity

In vivo, stem cell microenvironments are characterized by macromolecular crowding. In vitro, this feature is absent and thus stem cells lose scientific and therapeutic potential. Demonstrated herein is that re-introducing crowdedness with mixed macromolecular crowding (mMMC) at a biologically relevant volume fraction occupancy of 12-15% substantially improves the adipogenic differentiation response of human bone marrow-derived mesenchymal stem cells (hMSCs) compared to standard protocols (adipocyte yield, lipid content per cell). This amplification was brought about by the mMMC-enhanced deposition and supramolecular assembly of ECM components and subsequently enhanced differentiation stage-specific remodeling of the ECM. The biochemical cues contained in cell-free matrices formed under crowding were capable of driving naïve hMSCs into adipogenesis without chemical induction and induced lineage-typical CpG methylation patterns. This indicates that mMMC is not only an important biophysical driver for microenvironment creation, it also underlines the importance of matrix reciprocity in maintaining and modulating stem cell phenotypes.

Specifically shown herein is that MMC potentiates adipogenic differentiation yield and extent in human MSCs, Macromolecular Crowding (MMC) is a driver of cellular microenvironment formation and enhances deposition/remodeling of lineage-directing ECM by adipogenically differentiatiated MSCs, and a potentiating effect of MMC works via biochemical cues incorporated into generated ECM.

Mesenchymal stromal or stem cells (MSCs) are multipotent precursor cells in the bone marrow. Clinical applications of MSCs require ex vivo expansion to generate therapeutically relevant cell numbers, but extended propagation results in a loss of self-renewal capacity and multipotentiality (Banfi, A., et al., Exp Hematol, 6:707-715 (2000)). It is increasingly recognized that the microenvironment—including growth conditions and substrata—differ greatly from the original tissue microenvironments from which these cells are derived. MSCs reside in a physiologically crowded microenvironment composed of soluble factors, cellular components and dense arrays of solid extracellular matrix (ECM); which is crucial in maintaining their self-renewal, multipotentiality via cell fate determination and preconditioning of progeny daughter cells (DiGirolamo, C., et al., Br J Haematol, 275-281 (1999)). Maintenance of their phenotype or differentiation is governed by specific cues within each unique local microenvironment (DiGirolamo, C., et al., Br J Haematol, 275-281 (1999)). Current strategies aim to mimic the in vivo conditions by accounting for the cell-cell, cell-matrix and cell-growth factor interactions via gel systems, surface coatings of cell culture plastic and nano-texturing of cell culture supports (Blow, N, Nature Meth, 8:619-622 (2009)). However, the capacity of progenitor cells to build their own microenvironments has long been underutilized if not neglected. One reason for this is the apparent inefficiency of cells to produce large amounts of extracellular matrix within a useful time window (Lareu R R., et al., FEBS Lett, 14:2709-2714 (2007)).

As shown herein, the highly dilute aqueous conditions and lack of crowdedness of contemporary cell culture is a key reason for the apparent lack of efficiency for ECM formation in vitro (Lareu, R R, et al., FEBS Lett, 14:2709-2714 (2007)). With 5-20% fetal bovine serum content or special formulations for serum replacement and xeno-free media, the solute content does not reach the necessary level of fraction volume occupancy that would constitute a macromolecular crowded state (Ellis R J, Curr Opin Struct Biol, 1:114-119 (2001)). While the ECM provides macromolecular confinement, the interstitial spaces contain macromolecular solutes, which create an excluded volume effect (EVE) that is generated by the mutual impenetrability of solute molecules. The ECM molecules and the macromolecular solutes in the interstitial spaces occupy vast parts of a given volume and exclude like-sized molecules by steric exclusion and additional electrostatic repulsion. This high level of fractional occupancy has been shown to greatly influence equilibria and rates of biochemical reactions that depend on non-covalent associations and/or conformational changes, such as protein and nucleic acid synthesis, intermediary metabolism, cell signaling and gene expression (Ellis R J, Curr Opin Struct Biol, 1:114-119 (2001)). Even blood plasma shows a solute content of 80 mg ml and represents a macromolecularly crowded body fluid (Ellis, R J, Trends Biochem Sci, 10:597-604 (2001)). It has recently been shown that introducing negatively or neutrally charged macromolecules to a culture of fibroblasts, drives the deposition of extracellular matrix; which in turn enhances microenvironment formation as a result of enhanced conversion of procollagen to collagen (Lareu, R R, et al., Tissue Engineering, 13(2):385-391 (2007a), Lareu, R R et al., FEBS Letters, 581(14):2709-2714 (2007b), Chen et al., Br J Pharmacol, 5:1196-1209 (2009)), enhances supramolecular assembly of collagen (Chen et al. Adv Drug Deliv, in preparation) and enhances enzymatic crosslinking (Chen et al., Br J Pharmacol, 5:1196-1209 (2009), Chen et al. Adv Drug Deliv, in preparation).

As described herein, the crowdedness of the bone marrow in approximation was emulated by creating a defined volume fraction occupancy using mixed macromolecular crowding composed of a mixture of Ficoll70 and Ficoll400 with a hydrodynamic radius of ˜4 nm and ˜13 nm, respectively, resulting in a biologically relevant volume fraction occupancy of ˜45% on human bone-marrow derived mesenchymal stem cells with the intention to facilitate endogenous microenvironment building. Demonstrate herein is that MMC (e.g., mixed MMC) not only enhances the matrix formation of undifferentiated MSCs but also enhances the differentiation specific remodeling steps during chemically induced adipogenesis so that the microenvironment of the finally resulting adipocyte is not only significantly different in its ECM composition, but is quantitatively richer in its overall components. Under these conditions, MSC undergoing chemically induced adipogenesis respond by a tripling of individual lipid content of generated adipocytes, and simultaneously a 2.5 fold increase of cells that undergo differentiation at all. Moreover, it is shown that the resulting adipocyte matrix—as created under MMC—is adipogenic per se to undifferentiated MSCs even in the absence of chemical inducers. The combined results reveal that biochemical cues in the matrix give rise to cell-matrix reciprocity and demonstrate the importance of a rich and complex microenvironment.

### Materials & Methods

### Calculation for Emulating Bone Marrow Crowdedness

| | | |
|---|---|---|
| 1 | Calculate the Volume of each Ficoll 70 | Calculate the Volume of Each Ficoll 400 |
| | molecule considered as a sphere with a | molecule considered as a sphere with a radius |
| | radius of 4 nm (4e⁻⁹ m) at 37.5 mg/ml | of 8 nm (8e⁻⁹ m) (Harve, KS, Biophys Rev Lett |
| | (Harve, KS, Biophys Rev Lett 1(3): 317-325 | 1(3): 317-325 (2006)) |
| | (2006)) | |
| | Volume of a sphere = 4/πr³ = | Volume of a sphere = 4/πr³ = |
| | 4/3π (4e⁻⁹)³ = | 4/3π (13e⁻⁹)³ = |
| | 268 × 10⁻²⁷ cubic meter → 1 | 2144.66 × 10⁻²⁷ cubic meters → 1 |
| 2 | Calculate the number of Fc70 molecules | Calculate the number of Fc70 molecules in |
| | in 37.5 mg | 37.5 mg |
| | From literature it is known that the MW | From literature it is known that the MW of |
| | of Fc70 is ~70,000 Da | Fc400 is ~40,000 Da |
| | That means, 70,000 grams of Fc70 | That means, 400,000 grams of Fc400 contain |
| | contain Avogadro number of molecules | Avogadro number of molecules |
| | From this, the number of molecules of | From this, the number of molecules of Fc400 |
| | Fc70 present in 3.75 mg is calculated as | present in 25 mg is calculated as follows: |
| | follows: | (25 × 10⁻³) × (6.023 × 10²³)/400,000 = 3.76 × 10¹⁶ |
| | (3.75 × 10⁻³) × (6.023 × 10²³)/70,000 = | → 2 |
| | 32.2 × 10¹⁶ → 2 | |
| 3 | Calculate the Fraction volume Occupancy | Calculate the Fraction volume Occupancy in |
| | in 1 ml | 1 ml |
| | Now, the volume occupied by these | Now, the volume occupied by these number of |
| | number of Fc70 molecules in 1 ml is | Fc400 molecules in 1 ml is calculated by |
| | calculated by multiplying 1 & 2 and then | multiplying 1 & 2 and then expressing in |
| | expressing in percentage to estimate the | percentage to estimate the fraction volume |
| | fraction volume occupancy: | occupancy: |
| | 268 × 10⁻²⁷ × 32.3 × 10¹⁶ = 8.7% (v/v) | 2144.66 × 10⁻²⁷ × 3.76 × 10¹⁶ = 8% (v/v) |

Therefore in a Fc70-Fc400 mix, the total fraction volume Occupancy is 8.7 + 8 = 16.7%

### Mesenchymal Stem Cell Culture

Human bone-marrow derived mesenchymal stem cells (MSCs) were obtained commercially (Cambrex) at passage 2 (p2) and cultured in a basic culture medium composed of low glucose Dulbecco's modified Eagle's medium (LG DMEM, Gibco-BRL) supplemented with Glutamax, 10% fetal bovine serum, 100 units/ml penicillin and 100 μl/ml streptomycin. Cells were maintained at 37° C. in a humidified atmosphere of 5% CO₂/95% air, with medium change twice per week. To prevent spontaneous differentiation, cells were maintained at subconfluent levels prior to being detached using TrypLE™ Express (Gibco), passaged at 1:4 and cultured to generate subsequent passages. Directed differentiation was carried out with cells at passage 4 (p4) and 8 (p8).

### Adipogenic Induction of Mesenchymal Stem Cells (MSC)

MSCs at an early passage (p4) and late passage (p8) were seeded at an initial density of 3.5×10⁴ cells/well in 24-well plates (Celstar). Adipogenic differentiation was stimulated 16 hours post-seeding as described (Pittenger, mf ET AL., Science, 5411:143-147 (1999)) via three cycles of induction/maintenance, feeding with induction medium for 4 days (37° C., 5% CO₂); followed by 3 days of culture in basal medium. Non-induced control MSC will be fed only basal medium on the same schedule. Basal medium is composed of high glucose Dulbecco's modified Eagle's medium (HG DMEM, Gibco-BRL) supplemented with 10% fetal bovine serum, 100 units/ml penicillin, 100 μl/ml streptomycin and insulin (0.01 mg/mL) while induction medium is supplemented with 3-Isobutyl-1-methylxanthine (0.5 mM), dexamethasone (10-7M) and indomethacine (0.2 mM). Crowding is effected by the addition of a crowding cocktail during both the induction and maintenance phase.

### Nile Red Adherent Cytometry to Assess Area of Cytoplasmic Lipid Accumulation

After 21 days (corresponding to three complete induction cycles), monolayers in 24-well plates was rinsed with PBS, fixed in 4% formaldehyde (30 min; RT) then co-stained with Nile Red (Sigma-Aldrich; 0.05 mg/ml in water; 1 hr), a fluorescent lysochrome specific for cytoplasmic lipid droplets and 4′,6-diamidino-2-phenylindole (DAPI; 0.5 μg/ml; 30 min) as described previously (Greenspan, P., et al., J Cell Biol, 3:965-973 (1985)). Adherent fluorescent cytometry was based on a montage of 9 sites per well taken by a coolSNAP HQ camera attached to a Nikon TE2000 microscope at 2× magnification, covering 83% of total well area. Nile Red was viewed under a single rhodamine filter [Ex572 nm/Em630 nm] while DAPI fluorescence was accessed with a single DAPI filter [Ex350 nm/Em465 nm]. Measured Nile Red events were thresholded and measured by an image analysis software (MetaMorph 6.3v3). Extent of adipogenic differentiation was quantified by area of Nile Red fluorescence from thresholded events normalized to nuclei count based on detected DAPI fluorescence. End data correspond to total area of lipid droplets present per well relative to cell number.

Flow Activated Cytometry to Assess Percentage of Cells that have Differentiated

Monolayers were washed twice in PBS, trypsinized and pelleted by centrifugation for 5 min at 2000 rpm at room temperature. Resuspended pellets were stained with Nile red (0.05 mg/ml in water; 30 min) and fixed by adding formaldehyde (FA) to a concentration of 0.5% as described previously (Gimble et al., J Cell Biol, 3:393-402 (1995). The samples were then analyzed with a FACScan™ flow cytometer (Becton Dickinson, Basel, Switzerland). Nile Red fluorescence was measured on the FL2 emission channel through a 585±21 nm band pass filter, following excitation with an argon ion laser source at 488 nm. Using a forward scatter (FSC)/side scatter (SSC) representation of events, a gating region was defined to exclude cellular debris from the analysis. Using an overlay histogram (event count/FL2) with un-induced MSCs stained with Nile Red as background control (green), a bar region R2 was then established on the gated population (red) in order to count cells with high FL2 values (adipocytes). Data acquisition and analysis was performed using CyAn™ ADP (DAKO). For each sample, 10 000 events were collected. The results are expressed as the percentage of cells appearing in R2.

### Gene Expression and CpG Methylation Studies

Total RNA was extracted using the RNAeasy single step column spin following the manufacturer's protocol and used to synthesize cDNA using Superscript reverse transcriptase II. RT-PCR reactions were performed and monitored on a Stratagene real-time PCR instrument (Stratagene) with a PCR master mix based on Platinum Taq DNA polymerase (Fermentas). Data analysis was carried out with the MxPro software (Stratagene). For each cDNA sample, the Ct value will be defined as the cycle number at which the fluorescence intensity reached the amplification based-threshold fixed by the instrument-software. Relative mRNA expression level for peroxisome proliferator-activated receptor gamma (PPAR-γ) and leptin (Lp) was determined using the ΔΔ-Ct method with ribosomal protein (RLPO) levels as an endogenous control.

### Immunohistochemistry

Monolayers were fixed with 4% methanol-free formaldehyde (Pierce) for 10 mins at room temperature then blocked with 3% bovine serum albumin (Fluka05488) (BSA) in PBS. Immunofluorescence was carried out using primary antibodies mouse anti-human collagen I (SigmaC2456, 1:1000); rabbit anti-human fibronectin (DakoA0245, 1:100); rabbit anti-human collagen IV (Abcam6586, 1:100), Alexa Fluor 488 phalloidin (Invitrogen 1:40) at 1:40 and SelectFX Alexa Fluor® 488 Endoplasmic reticulum labeling kit (Invitrogen 1:1000). Secondary antibodies used were AlexaFluor 594 goat anti-mouse (Molecular Probes, A11020) at 1:400 dilution and AlexaFluor 488 chicken anti-rabbit (Molecular Probes, A21441) at 1:400 dilution. Cell nuclei were counterstained with 0.5 μg/ml 4′,6-diamidino-2-phenylindole (Molecular ProbesD1306) (DAPI). Images were captured with an IX71 inverted fluorescence microscope (Olympus).

### Protein Extraction and Western Blotting

Medium was harvested and washed cell layers were extracted with a buffer containing 150 mM NaCl, 50 mM Tris (pH 7.5), 5 mM EDTA (pH 8.0), 1% Triton-X100, and Protease Inhibitor Cocktail tablets (Roche Diagnostics Asia Pacific, Singapore). Subsequently, 20 uL of protein extract (even cell numbers) for each sample were mixed with 1× Laemmli buffer and 2 mL of b-mercaptoethanol and subjected to small format SDS-PAGE as described in the preceding text. Proteins were then electroblotted onto a nitrocellulose membrane (Bio-Rad) with the Mini Trans-Blot transfer cell (Bio-Rad) according to the manufacturer's protocol. Membranes were blocked with 5% nonfatmilk (Bio-Rad) in TBST pH 8 (20 mM Tris-base—150 mM NaCl—0.05% Tween-20) for 1 h at RT. Subsequently, the primary antibody (rabbit anti-human collagen IV; abcam) at a 1:500 dilution with 1% nonfat milk in TBST was incubated for 1 h at RT. Bound primary antibody was detected with goat anti-rabbit HRP (Pierce Biotechnology, Rockford, Ill.) diluted 1:1000 in 1% nonfat milk in TBST for 1 h at RT. The membrane was then incubated with Super Signal West Dura substrate (Pierce Biotechnology) and chemiluminescence was captured via an LAS-1000 Luminescent Image Analyzer (Fuji Photo Film, Tokyo, Japan).

### Deposition and Decellularisation of Matrices (Lysis)

To generate the lineage-specific matrices-MSC Matrix; MSCs were seeded at 3.5×10⁴ cells/well in 24-well plates (Celstar) and maintained in basal media ±crowder for 7 days prior to lysis. Preadipocyte matrix; MSCs were seeded at 3.5×10⁴ cells/well in 24-well plates (Celstar) and underwent 1 cycle of adipogenic induction/maintenance ±crowder for 7 days prior to lysis. Adipocyte matrix; MSCs in T175 flasks were adipogenically induced for 2 weeks prior to being seeded at an density of 3.5×104 cells/well in 24-well plates (Celstar) and treated to 1 cycle of adipogenic induction/maintenance ±crowder for 7 days prior to lysis. For lysis, monolayers were first washed with PBS twice then treated with 0.5% DOC (Prodotti Chimici E Alimentari, S.P.A. 2003030085) supplemented with 0.5× Complete Protease Inhibitor (Roche Diagnostics Asia Pacific, 11836145001) in water for 15 minutes at room temperature four times, followed by 0.5% DOC in PBS for 30 minutes at RT on a nutating platform. Monolayers were then incubated with DNAse (Sigma) for 1 hr at 37° C., then washed with PBS three times. For salt depleted matrices, two washes with NaCl (5M) prior to DNAse treatment were performed.

### Statistical Analysis

All assays were performed in triplicate and results reported as means±standard deviation. Independent sample t tests were used to determine statistical significance; the significance for all tests was set at p<0.05. (n=3; error bars are ±s.d.; ** P<0.01; * P<0.05).

### Results

### Macromolecular Crowding Increases The Yield And Extent Of Adipogenesis

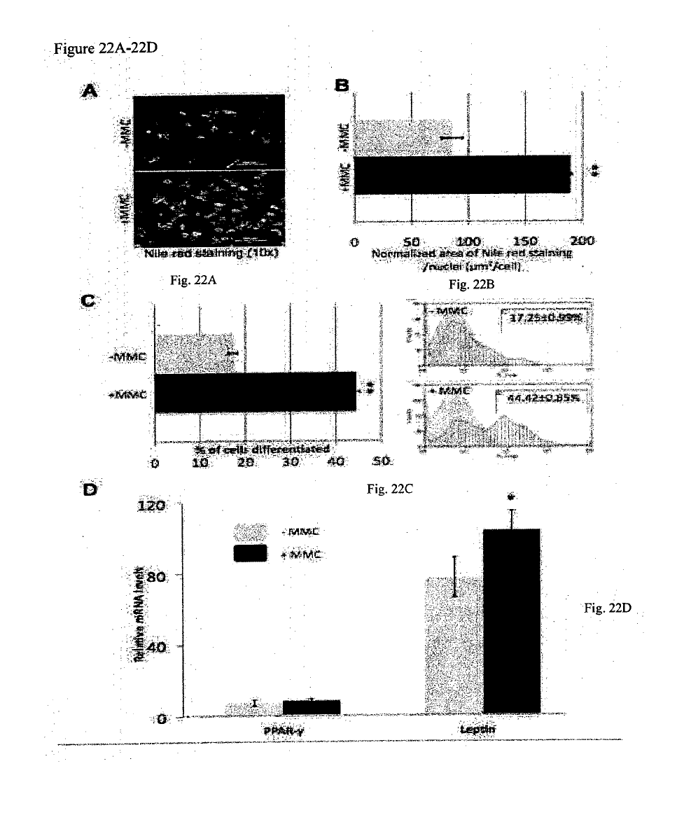

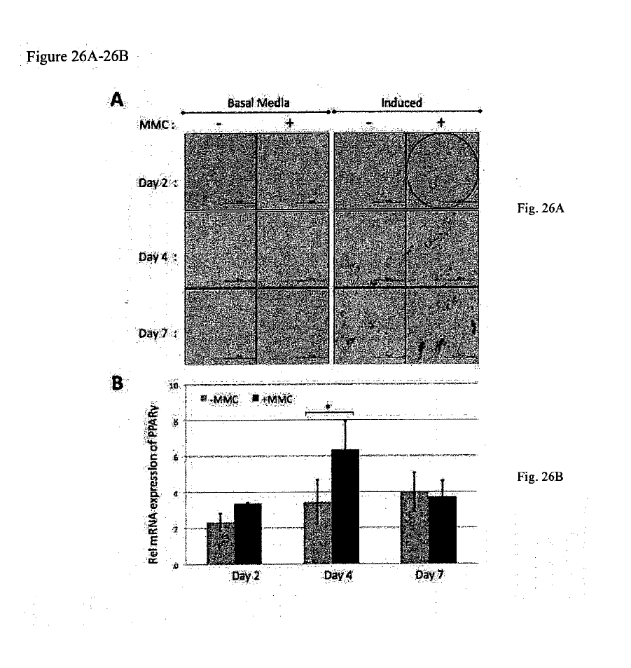

Induced differentiation of hMSCs toward adipogenesis resulted in a larger cell morphology and a time-dependent increase in intracellular lipid-filled droplets. After 21 days of induction, hMSCs should have fully differentiated and attained a matured phenotype defined by an accumulation of cytoplasmic lipid droplets (Gregoire, F M, et al., Physiol Rev, 3:783-809 (1998)). Based on Nile Red staining, a specific lipophilic dye (FIG. 22A), it was observed that monolayers exhibited a higher accumulation of lipid droplets when differentiated in the presence of MMC as compared to control un-crowded cultures. Normalizing the area of Nile Red stained lipid droplets to cell numbers by adherent fluorescent cytometry, it was found that MMC caused a 2.2 fold increase in normalized nile red stained area from 85.6±9.24 μm²/cell to 188.82±2.25 μm²/cell (FIG. 22B). This indicated that each differentiated cell contained more lipid under MMC compared to control un-crowded cultures, which translated to more a matured differentiated phenotype. Next, it was determined whether MMC was also able to increase the proportion of cells that exhibit these droplets, as there is low efficiency in differentiation especially with cells obtained from older donors (Stenderup, K, et all. Bone 6:919-926 (2003)). Flow cytometry data obtained at end of 21 days (FIG. 22C) shows that treatment of confluent cultures with induction media in the absence of MMC (ctrl) resulted in 17.25±0.99% of the population becoming adipocytes, while under MMC there was a 2.57 fold increase as 44.42±0.85% of the population exhibited lipid droplets. This demonstrates that MMC not only enabled cells to attain a more matured differentiated phenotype (FIGS. 22A-22B) but also that more undergo differentiation (FIG. 22C). Gene expression analysis also confirms that the monolayers differentiated under MMC exhibit a higher level of the lipogenic genes PPAR-γ and leptin (FIG. 22D) confirming that the cells have indeed undergone more adipogenesis. It was also observed that MMC resulted in an earlier onset of adipogenesis (FIG. 26A). Earlier morphological changes and the presence of lipid droplets in induced cultures at day 2 under crowded conditions (+MMC, circled in red) was also observed. In contrast, no lipid droplets are visible in the non-crowded condition (−MMC). By day 4, there were more lipid droplets under crowded conditions (+MMC) versus non-crowded (MMC). At day 7, lipid droplets under crowded conditions were larger than those under non-crowded conditions, indicating a more matured phenotype in the presence of crowding compared to non-crowded conditions. Monolayers that were maintained in basal media in the presence or absence of MMC did not express any lipid droplets at all timepoints, confirming that the ficoll cocktail had no adipogenic inductive properties (FIG. 26A, column 1 &2). In addition, it was found that the relative expression of the master adipogenic regulator gene, PPARγ (FIG. 26B) was elevated at day 2 and 4 under MMC.

### MMC Enhances Deposition and Remodeling of the Extracellular Matrix (ECM)

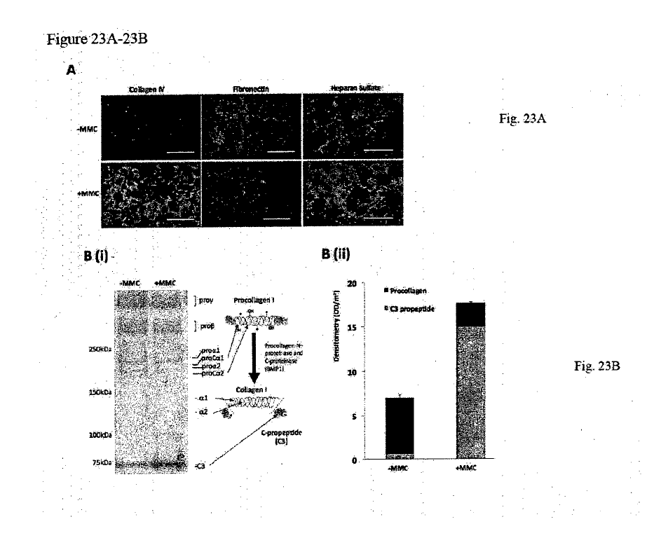

The extracellular matrix (ECM) as a component of the microenvironment plays an important role in directing the differentiation and maintaining the phenotype of cells (Fuchs, E et al., Cell, 6:769-778 (2004), Bissel, M J et al., Prog Clin Biol Res: 251-262 (1987)). Whether MMC is affecting the deposition and remodeling of the ECM proteins specifically involved in the adipogenic differentiation pathway was examined by comparing structure and quantity deposited in the absence and presence of crowding by adipogenically induced hMSCs. From FIG. 23A, a morphological change is seen occurring with fibronectin and collagen IV deposited on the cell layer—possessing a more fibrillar pattern in the hMSCs and a more reticular morphology in the mature adipocytes, as has been described previously (Greogoire, F M, et al., Physiol Rev, 3:783-809 (1996)). For collagen IV, a general increase in deposition in the presence of MMC was seen. While conversely, an increased degradation occurring with fibronectin is seen as has been described to occur during adipogenic matrix remodeling (Lilla, J., et al., Am J Pathol, 5:1551-1554 (2002)). Sulfated polysaccharides such as heparin and heparan sulfate are associated with the extracellular matrix. The monolayers were assessed for the presence of heparan sulfate (HS) and confirmed an increase in staining under macromolecular crowding (+MMC)

### MMC Enhances the Proteolytic Processing of Collagen I

It has been previously demonstrated that MMC with a negative crowder; dextran sulfate, enhanced the proteolytic processing of collagen I in fibroblast cultures resulting in enhanced collagen deposition (Lareu, R R., et al., Tissue Eng, 2:385-391 (2007)). To assess if a similar enhancement in procollagen processing was occurring in cultures with a neutral Ficoll cocktail, immunoblotts were prepared for procollagen C-propeptide in medium harvested from MSC and adipocyte cultures after 8 days in culture in the absence or presence of MMC (FIG. 23B(i)). An increase in cleaved C-propeptide was observed for samples with MMC accompanied with a corresponding decrease in the procollagen fragments. Densitometry of the C3 bands confirms a xx fold increase in C3 fragment in cultures under MMC (FIG. 23B(ii))

### MMC Enhances the Amount of Heparan Sulfate-Associated Ligands

As shown in FIG. 23A, there is an increase in heparan sulfate, and because these polysaccharides have been shown to interact and sequester a vast array of growth factors within the ECM it was decided to assess if this increase in heparan sulfate corresponds to an increase in its associated growth factors.

### Matrices Affect Potency Of Adipogenic Induction Cocktail

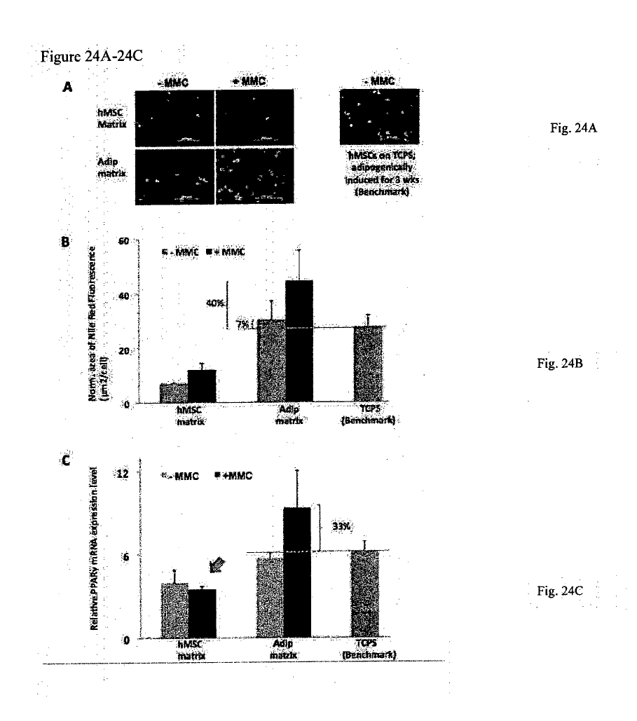

The substrate upon which naïve hMSCs are plated affects their adipogenic differentiation efficiency (Mauney J R, et al., Biomaterials 16:3233-3234 (2004), so it was examined whether the decellularised matrices derived from hMSCs (hMSC), preadipocytes (Preadip) and matured adipocytes (Adip) that were deposited in the absence (−MMC) and presence of crowding (+MMC) would have any effect on adipogenic induction of naïve hMSCs for 21 days (in the absence of crowding). As a benchmark, hMSCs were also induced on TCPS (FIG. 24A). It was observed that the hMSCs reseeded and cultured under adipogenic induction on adipocyte matrix (deposited under +MMC) exhibited the most lipid droplets, followed by those seeded on adipocyte matrix (deposited under −MMC) (FIG. 24A). Absence of Nile Red staining in the matrix-only wells (Figure not shown) confirmed that false positive from the substrate were not occurring. The visual observation from FIGS. 24A-24B was confirmed by adherent cytometry, which provides an overall analysis of the area of cytoplasmic lipid normalized to cell numbers within the whole well (FIG. 24B). From the results (FIG. 24B), it was observed that decellularised adipocyte matrices (−MMC) and (+MMC) were better substrates for naïve hMSCs undergoing exogenous adipogenic differentiation compared to the conventional method of inducing them on TCPS, by about 7% and 40%; respectively. In agreement to adherent cytometry data (FIG. 24B), a similar trend was observed at the gene level (FIG. 24C) as the relative expression level of PPARγ in hMSCs reseeded on adip matrix (+MMC) were about 33% higher than those seeded on TCPS and lower for those reseeded on hMSC and preadipocyte matrices. Macromolecular crowding (MMC) is also observed to further potentiate this effect, where hMSC matrix (+MMC) and preadip matrix (+MMC) generate stronger signals to counteract the chemical stimulus inducing adipogenesis, therefore resulting in a lower PPARγ expression than those reseeded on hMSC matrix (−MMC) and preadipocyte matrix (−MMC), respectively.

Decellularised Matrices from Adipocytes have Adipogenic Potential that is Sensitive to Salt Elution

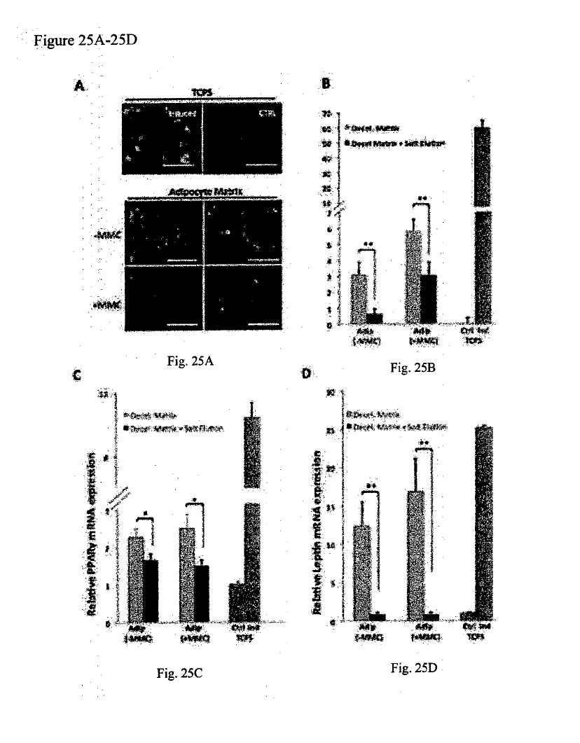

Recently, decellularised lung tissues have been described to direct the differentiation of human embryonic stem cells (Cortiella J, et al., Tissue Eng Part A, 16(8):2565-2580 (2010)), and better adipogenic differentiation was observed under chemical stimulus of naïve hMSCs on adipocyte matrices compared to TCPS (FIG. 25A). This led to the investigation of the lineage-directing potential in the absence of chemical stimulus of the extracellular matrices deposited by adipogenically induced hMSCs (adip) in the absence (−MMC) and presence of macromolecular crowding (+MMC). To this end, naïve hMSCs were seeded on the various matrices and maintained in basal medium for 3 weeks. By day 21, the cells showed different degrees of spontaneous adipogenesis dependent on the degree of differentiation of the cells that originally deposited the matrices. This effect was intensified in correlation to the enhancement of ECM deposition under MMC. Lipid droplet content as the most advanced marker was most pronounced with matrices made by adipogenically induced hMSCs under MMC both optically (FIG. 25A) and after quantitative cytometry (FIG. 25B). The normalized Nile Red stained area was 5.8 μm2/cell which represents 10% of the values obtained with cells adipogenically differentiated on plastic (FIG. 25B). After three weeks, the matrices induced a significant elevation of PPARγ mRNA and the overall increase of PPARγ-mRNA amounted to 20-25% of expression levels achieved with classical adipogenic differentiation on plastic (FIG. 25C). In contrast, leptin mRNA levels showed a striking correlation and ranking order [ctrl TCPS <<adip −MMC <<17 fold adip +MMC <25 fold TCPS induced]. In this case the highest expression of leptin mRNA—on adipocyte ECM+MMC—represented around 70% of the levels after adipogenic induction on plastic (FIG. 25D). In order to address the question whether the deposited ECMs contained elution-sensitive ligands that would induce adipogenesis the deposited matrices were treated with 0.5 M sodium chloride (0.5M NaCl). In all instances, sodium chloride treatment significantly reduced adipogenesis induction, moderately for PPARγ mRNA induction, strongly in lipid droplet content, and dramatically for leptin mRNA expression. PDRM16 is a critical gene involved/upregulated during adipogenesis (Ravussin, E., et al., Obes Rev, 10(3):265-268 (2009)). The methylation pattern in two loci on the PDRM16 gene in MSCs adipogenically induced in the absence and presence of crowding were compared with those obtained in naïve MSCs exposed to cell-free matrices produced by adipogenically induced cells in the presence/absence of crowding and kept on these matrices for 7 d in plain culture medium. The aim of this analysis was to see whether the epigenetic effects brought about by the classical continued chemical lineage induction would be emulated by sheer exposure of undifferentiated MSC's to cell-free lineage-derived ECM's. Interestingly, not only was the trend of decreased methylation upon prolonged ECM contact identical to that seen with chemical differentiation undercrowding, but also more pronounced. As a decrease in methylation corresponds to an upregulation in gene expression (Gene Regulation. A Eukaryoti Perspective, Third ed. Latchman D S, Stanley Thomas (Pub.) Ltd (1998)) it is inferred that the matrices deposited under crowding are indeed a strong driver of differentiation and are most likely the largest contributing factor to the MMC-mediated augmentation of chemical differentation.

### Discussion

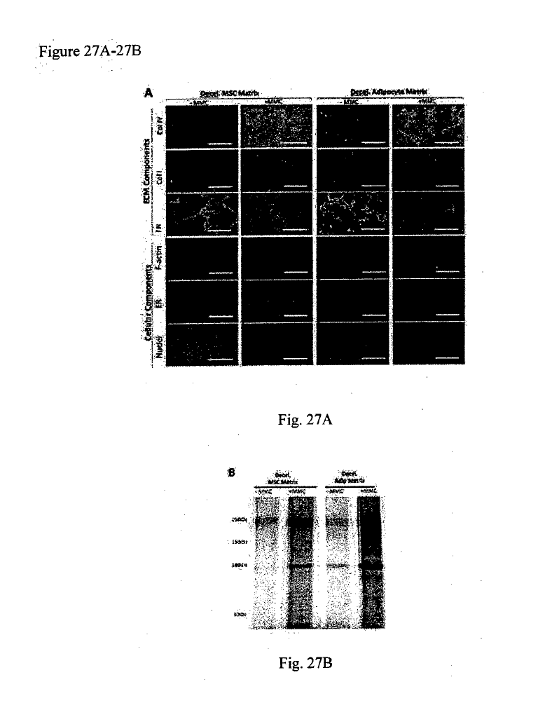

Macromolecular crowding is a well researched phenomenon in physical chemistry and biophysics and studied in the realm of protein folding (Cheung, M S, et al., PNAS 102:4753-4758 (2005)) and nucleic acid hybridisation (Harve, K S, et al., Nucleic Acis Res (epub Oct. 23 2009). Although it is an ubiquitous parameter of life functions MMC is surprisingly understudied and underutilised in biological systems. Described herein is the successful generation of a degree of crowdednesss for MSCs in vitro modelled after some aspects of their physiological microenvironment in the bone marrow. Because in vivo, there is never a single dominant crowding molecule in a given volume, mixed MMC was chosen. Mixed MMC has been shown in protein folding studies (Cheung et al., PNAS, 102:4753-4758 (2005)) to be more efficacious than mono-crowding. Indeed, shown herein is that with neutral crowders mixed MMC is more efficacious than with neutral crowders (Chen, CZC, et al., Br J Pharamcol, 5:1196-1209 (2009); Chen, C., et al., Adv Drug Delivery 2011 in preparation), in addition the collagen and fibronectin matrices formed under neutral crowding would form a more meshwork-like structure instead of multiple aggregates as seen with negative crowders ((Chen, CZC, et al., Br J Pharamcol, 5:1196-1209 (2009)). Using currently published ranges of biologically active fraction volume occupancies that are created by macromolecular crowders, a mixture of neutrally charged Ficoll 70 and 400 was chosen and optimized because a volume fraction occupancy of 12-15% was obtained (see materials & methods for calculations). Other considerations were that these macromolecules do not increase viscosity of culture media, which is crucial for bioreactor-based work, and these crowders are already in use for clinical preparations of mononuclear cell fractions of peripheral blood. Based on the lack of published information on the biophysical parameters of the bone marrow and calculations, serum albumin was chosen as main representative of plasma macromolecules. While serum albumin was only a pars-pro-toto and while this approach was only approximate, the novel culture composition greatly facilitated MSCs to finish building and remodeling their own microenvironments. A surprisingly strong and fast response of ECM assembly by MSC's as exemplified by rapid fibronectin assembly, conversion of procollagen I to collagen I, deposition of collagen IV and striking increase in heparansulfate proteoglycans deposition, was observed. The data provided herein is an optimum of ECM deposition that can be achieved by MSCs in static culture. Interestingly, under MMC the adipogenic differentation response of MSC's showed not only a tripling of the lipid content per differentiated cell but 2.5 fold more differentiation yield, i.e. more cells actually became lipid droplet bearing cells under chemical induction. These differentation yields represent a major breakthrough for reducing noise for genetic, epigenetic and metabolic studies of adipocytes in vitro. While the same system appears to work very well for osteogenesis (unpublished data), chondrogenic differentiation of the same cells still requires pellet culture (not shown). It is likely that the passages of MSCs used were already too far pre-committed. It is interesting to see the combined expression of collagen I and IV on the protein level by undifferentiated MSCs. For osteogenesis this would require only to down-regulate transcription or downstream regulation of translation of the collagen IV message, and for adipogenesis, and as shown herein, this would require the downregulation of collagen I and upregulation of collagen IV and appropriate remodeling. Chondrogenesis, however, would require a new collagen type II to be over-expressed and to override a pre-existing large amount of collagen I. In order to study the mechanism by which mMMC does enhance differentiation, the deposited ECM was studied for its lineage-directing capacity. Demonstrated herein is that the ECM enhancement drives the adipogenic differentiation and works as the amplifier of the differentiation signal. Using ECM decellularised via detergent treatment (see FIGS. 27A-27C), it is shown herein that lineage-directing information is retained in these matrices, in particular if they have been laid down under MMC-enhancement. The matrices were able to increase the level of mRNA expression of sentinel genes of adipogenesis. PPARγ, an early marker showed a general increase that appeared to be independent however, the overall leptin mRNA induction reached almost 70% of that of classical induced hMSCs. This is remarkable given the fact that the matrices were decellularised rather harshly leaving to substantial losses of ECM proteins and ligands. In terms of overall lipid content naïve hMSCs showed after 3 weeks exposure to these matrices 10% of the content in comparison to cells classically induced with a differentiation cocktail on plastic, while non-induced cells on plastic were completely negative. Lipid droplet content is a very late marker for a fully differentiated adipocyte and it might be that a 3 weeks observation period cells were more slowly differentiating in comparison to those induced. However, a look at the leptin mRNA levels showed that the cells exposed to lineage-directing matrices only were well on their way to become adipocytes as evidenced by their impressively upregulated mRNA levels. Altogether, the data are remarkable, as they have been generated by a harsh non-ionic detergent lysis of the cells and thus losses of ECM components and respective ligands during the dissolution of cell membranes are inevitable. In fact, ECM losses after DOC lysis is estimated to be in the range of 90%. Nevertheless, the ECMs generate via DOC lysis retained lineage directing information, and a substantial part of this information can be removed from these matrices by neutral salt elution suggestion non-covalently bound molecules, such as growth factors.

### Example 4

### Human Fibroblast Matrices Bioassembled Under Macromolecular Crowding Maintain Human Embryonic Stem Cell Pluripotency

Feeder-free culture and propagation of human embryonic stem cells (hESCs) without losing pluripotency and proliferative capacity represents the current challenge for their clinical application. The murine sarcoma-derived extracellular matrix (ECM) Matrigel is widely used as cell-free support in combination with conditioned or chemically defined medium. However, inherent xenogenic and immunological threats invalidate this material for clinical application. Employing human fibrogenic cells to build ECM, however, has been hampered by inefficient and time-consuming deposition in vitro. As shown herein, applying macromolecular crowding creates an excluded volume effect, which in turn accelerates ECM deposition substantially. This system was used to deposit human ECMs at very low serum conditions in two days, by Wi-38 fetal human lung fibroblasts, using dextran sulfate 500 kDa. After decellularisation, these deposited matrices allowed the propagation of H9 hESC in chemically defined culture medium with an overall better outcome than Matrigel in terms of population doublings without loss of pluripotency and differentiation capacities. Notably, only ECMs generated under macromolecular crowding allowed the successful propagation of hECs and they were highly complex and contained, in contrast to Matrigel, collagen XII, ig-H3 and substantial amounts of transglutaminase 2. Genome-wide analysis of DNA methylation states revealed a high overall similarity between human ECM- and Matrigel-cultured hESCs, however distinct differences of 49 genes with a variety of functions were present. Taken together, human ECMs deposited by selected fibroblasts under macromolecular crowding represent a suitable human microenvironment for hESC propagation.

Successful propagation of hESCs in culture pursues two apparently contradictory aims, namely increasing the cell numbers in a culture device while preventing differentiation to the utmost level in order to preserve hESC pluripotency. Pioneering work by Ariff Bongso made it possible to propagate hESCs using a co-culture system (Bongso A, et al. Hum Reprod, 9(11): 2110-2117 (1994)). Originally, epithelia from human reproductive organs were used, and for reasons of practicality, inactivated murine embryonic fibroblast (MEF) feeders. The notion that MEFs and culture ingredients of animal origin harbour contamination risks expression of an immunogenic non-human sialic acid on the so cultured hESCs (Martin M J, et al. 2005, Nat Med, 11(2): 228-232) intensified the search for human feeder fibroblasts (Richards M, Bongso A. 2006, Methods Mol Biol, 331: 23-41). It turned out that not every cell line was a suitable feeder (Richards M, Bongso A. 2006, Methods Mol Biol, 331: 23-41). Furthermore, co-culture of hESCs with feeders in general creates noise in hESCs analyses, and feeder layers admixtured to hESC populations represent a regulatory obstacle. Therefore, feeder-free culture systems have become a priority in hESC work. It was therefore logical to look into MEF-derived ECM and human fibroblast-derived ECM for the propagation of hESCs (Xu C, et al. 2001, Nat Biotechnol 19: 971-974; Klimanskaya I, et al. 2005, Lancet 365: 1636-1641; Meng G, et al. 2010, Stem Cells Dev 19(4): 547-556), instead of using complete live cells.

However, while the in vitro production of ECM components does not pose a problem, their efficient supramolecular assembly and stable deposition as a matrix including crosslinking certainly is (Lareu, R R et al. Tissue Engineering, 13(2):385-391, (2007a)). Therefore, the generation of ECMs either from MEFs or human cells is largely inefficient and requires culture times of up to 21 days using 10% FBS or 10% human serum, respectively (Klimanskaya et al. Lancet, 365:1636-1641 (2005); Meng G, et al. 2010, Stem Cells Dev 19(4): 547-556)). FBS poses a xenogenic threat; therefore its replacement with human serum is a breakthrough towards xeno-free culture systems for hESCs. However, human serum is in limited supply and requires rigorous quality control, because human pathogens might be even more debilitating than xeno-pathogens (Mallon, B S, et al., J Biochem and Cell Biol., 38:1063-1075 (2006)).

As an intermediate solution, Matrigel is widely used as a commercially available ECM for the propagation of hESC, preferably in MEF-conditioned culture medium or in chemically defined media (Xu C, et al. 2001, Nat Biotechnol 19: 971-974; Ludwig T, et al. Nat Biotechnol 24(2): 185-187 (2006)). Matrigel is reconstituted from basement membrane ECM proteins secreted by a murine sarcoma cell line (Kleinman H K, et al. 1981 Biochem 21: 6188-6193). Because it lacks structures typical for native basement membrane, it is unclear to what extent this material can emulate a native microenvironment for hESCs. Obviously, Matrigel poses a xenogenic threat, thereby preventing the clinical applications of Matrigel-cultured hESCs. In addition, the exposure of hESCs to Matrigel induces them to express a non-human sialic acid Neu5Gc, which can cause an immune response in human hosts (Lanctot P M, et al. 2007 Curr Opin Chem Biol 11: 373-380).

Obviously, the unmet need persists to develop human cell-derived matrices that could serve as a native microenvironment for hESCs, can be rapidly produced, and requires minimal or no amounts of serum. It has been shown previously that the addition of 500 kDa dextran sulfate (DxS) (Lareu R R et al. 2007a Tissue Eng 13(2): 385-391); Lareu R R et al. 2007b FEBS Letters 581:2709-2714) or a mix of 70 kDa and 400 kDa Ficoll (Fc) (Chen CZC and Raghunath M. 2009a, Fibrogenesis & Tissue Repair 2:7) leads to enhanced and accelerated ECM deposition under low serum conditions. Described herein is the use of this technology to generate human bioassembled matrices that are growth supports for pluripotent hESC culture in conjunction with culture medium, e.g., mTESR-1, a chemically defined culture medium.

### Materials and Methods

### Preparation of Cell-Free Matrices

Fibroblast cell culture was as previously described (Lareu R R et al. 2007a Tissue Eng 13(2): 385-391); Lareu R R et al. 2007b FEBS Letters 581:2709-2714; Peng Y, Raghunath M. 2010 ‘Chapter 5: Learning from nature: emulating macromolecular crowding to drive extracellular matrix enhancement for the creation of connective tissue in vitro’ in Tissue Engineering, ed. Eberl D, IN-TECH, Croatia). Wi-38 fibroblasts (ATCC) at passage 20-22 were seeded at 25,000 cells/cm² in culture plates or flasks (Greiner Bio-one) under standard conditions in 10% FBS in DMEM. On the following day, culture medium was changed to 0.5% FBS (Gibco) medium containing 30 μg/ml ascorbic acid (Wako) with or without 100 μg/ml DxS (US Biologicals) or 37.5 mg/ml Fc 70 kDa and 25 mg/ml Fc 400 kDa. After 3 days (DxS) or 6 days (Fc), fibroblasts were lysed with a deoxycholate (DOC) (three washes), DOCDOC (six washed) or NP40 lysis protocol. DOC lysis protocol consists of three 10 minutes incubation on ice with 0.5% DOC (Prodotti Chimici E Alimentari S.P.A.) and 0.5× Complete Protease Inhibitor (Roche). DOCDOC protocol consists of six 10 minutes incubation on ice of 0.5% DOC and 0.5× Complete Protease Inhibitor. NP40 protocol is four 10 minutes incubation on ice of 1% Nonidet P-40 with 0.5× Complete Protease Inhibitor and two 30 minutes incubation at 37° C. of 1× DNase (US Biological). Following lysis, all matrices were washed with PBS 3 times and stored at 4° C. 293T human embryonic kidney cells and NT2 neuron-committed teratocarcinoma cells are seeded at 50,000 cells/cm² each in culture plates or flasks in 10% FBS in DMEM. Culture medium was changed to 0.5% FBS in DMEM supplemented with 30 μg/ml AcA and 100 μg/ml DxS on the following day. After 3 days of incubation, the cells were lysed using the DOC protocol as described earlier and the resulting matrices stored in PBS at 4° C.

hESC Culture

Frozen H9 (WiCell) at passage 25 were obtained from WiCell Research Institute and initiated onto MEF feeders for recovery. After 5 passages on MEFs, the hESCs were passaged using collagenase IV (Gibco) or dispase (StemCell Technologies) onto Matrigel (BD), DxSDOC and DxSDOCDOC matrices and cultured using mTeSR-1 (StemCell Technologies) with daily changes and subcultured every 5-7 days. Representative wells were sacrificed for cell counting by hemacytometer at each passage to calculate population doublings.

Characterization of hESCs

Antibodies used for flow cytometry using CyAn (DakoCytomation) and adherent immunofluoresence are: rat anti-SSEA-3 (Chemicon), mouse anti-SSEA-4 (Chemicon) and Alexa Fluor 488 chicken anti-mouse (Invitrogen) and Alexa Fluor 488 goat anti-rat (Invitrogen). goat anti-Oct-4 (Santa Cruz), mouse anti TRA-1-60 (Chemicon), mouse anti TRA-1-81 (Chemicon), Alexa Fluor 488 donkey anti-goat (Invitrogen), Alexa Fluor 594 goat anti-mouse (Invitrogen) and DAPI.

For in vivo teratoma formation assays, hESCs cultured for 18 passages were harvested using collagenase IV and were injected intramuscularly into the right hind limb of SCID mice. Neural differentiation in vitro was done using standard protocol from WiCell Research Institute. hESCs were harvested using collagenase IV and seeded onto non-adherent plates (Corning) to form embryoid bodies using 20% Knockout Serum (Invitrogen) in Knockout DMEM (Invitrogen). After 4 days, culture medium was changed to DMEM/F12 (Invitrogen) supplemented with Non-essential amino acids (Invitrogen), N2 (Invitrogen), heparin (Sigma) and FGF-2 (ProSpec) and embryoid bodies were seeded onto standard 6 well plates for 3 days. Embryoid bodies were seeded onto laminin (Sigma)-coated wells and for 7 days. Samples were formaldehyde-fixed and immunolabeled with rabbit anti-β III tubulin (Abeam) and Alexa Fluor 488 chicken anti-rabbit (Invitrogen) and DAPI.

Karyotyping services were obtained from KK Women's and Children's Hospital, Singapore. hESCs were harvested for DNA methylation by scraping and pelleted by centrifugation and snap frozen in liquid nitrogen. DNA methylation analysis was performed as described previously (Gronniger E, et al. 2010 PLoS Genet. 6(5): e1000971). Primer sequences for 454 bisulfite sequencing of the OCT4 promoter region were adapted from published primers (Deb-Rinker P, et al. 2005 J Biol Chem 280(8): 6257-6260). Genome-scale methylation profiles were obtained by using HumanMethylation27 BeadChips (Illumina). DNA methylation levels were scored as 13 values ranging from 0 (unmethylated) to 1 (completely methylated). Genes were scored as differentially methylated if their 13 value differed by 0.2 or more (Gronniger E, et al. 2010 PLoS Genet. 6(5): e 1 000971).

### Characterization of Cell-Free Matrices

Antibodies used for characterizing matrices are: mouse anti-PDI (Invitrogen), Alexa-Fluor 594 phalloidin (Invitrogen), mouse anti-collagen I (Sigma), rabbit anti-fibronectin (Dako), mouse anti-heparan sulfate (Seikagaku), mouse anti-fibrillin-1 (Chemicon), rabbit anti-decorin (gift from Dr Larry Fisher, LF-136), rabbit anti-LTBP-1 (gift from Dr Carl Heldin, AB39), Alexa Fluor 594 goat anti-mouse (Invitrogen), Alexa Fluor 488 chicken anti-rabbit (Invitrogen) and DAPI (Invitrogen).

### Results

Generation of NoDxSDOC, DxSDOC, DxSDOCDOC, DxSNP40 and FcNP40 matrices.

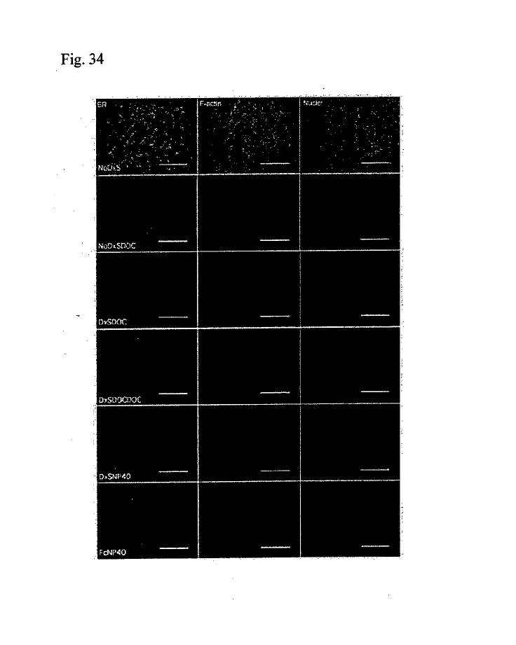

Wi38 fibroblast cell layers that were treated with only ascorbic acid (NoDxS), and DxS or Fc were subjected to either detergent treatment DOC, DOCDOC or NP40, to obtain NoDxSDOC, DxSDOC, DxSDOCDOC, DxSNP40 or FcNP40 matrices. Immunostaining confirmed the loss of intracellular structures, including endoplasmic reticulum, F-actin and nuclei (FIG. 34), thus demonstrating the successful generation of cell-free matrices. Matrices were also generated using 293T cells or NT2 cells and subjected to DOC lysis, resulting in 293TDOC and NT2DOC matrices.

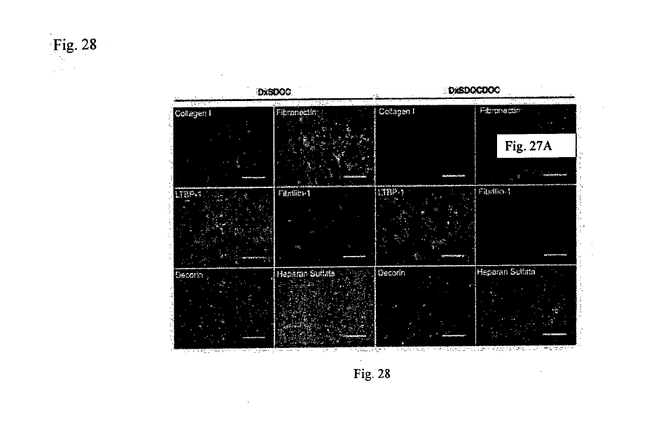

Although the removal of the cell layers brought about losses of associated ECM proteins, the resulting DxSDOC and DxSDOCDOC matrices continued to retain their constituent complexity as revealed by immunofluorescence (FIG. 28) and mass spectrometry. In DxSDOC matrices, collagen I was identified as granular staining, but was barely detectable in DxSDOCDOC matrices. Fibronectin was present in both matrices in heterogenous distribution, although DxSDOC matrices showed more fibronectin aggregates. The colocalization of collagen I staining and fibronectin showed the preserved interaction between collagen I and fibronectin after detergent treatments. LTBP-1 was present in apparently equal quantity and morphology in both ECM types. The microfibrillar component fibrillin-1 was found in DxSDOC matrices as large aggregates of irregular shapes, but was undetectable in DxSDOCDOC matrices. Both matrices showed a speckled deposition pattern for decorin, with weaker intensity in DxSDOCDOC matrices. Heparan sulfate proteoglycans were found in DxSDOC matrices as fine aggregates and in DxSDOCDOC as larger, more diffuse aggregates.

Mass spectrometry of the matrices found the presence of extracellular matrix proteins such as collagens, EMILIN-1, fibronectin, fibrillin I, tenascin, secreted protein transglutaminase 2, remnants of detergent lysis such as cytoplasmic heat-shock protein 70 kDa, cytoskeletal proteins actins and tubulins and nucleus-associated proteins such as histones.

293TDOC, NT2DOC, NoDxSDOC, DxSNP40 and FcNP40 Matrices do not Sustain Pluripotent Morphology.

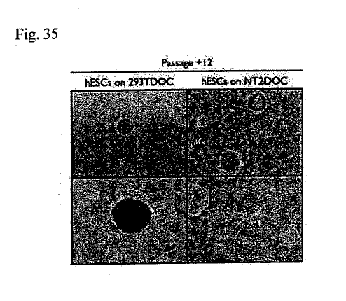

As an initial step towards the characterization of the human cell-derived matrices, the morphology of hESCs cultures on the above-mentioned matrices were analyzed. hESCs cultured on 293TDOC and NT2DOC matrices exhibited differentiated morphologies after 12 passages (FIG. 35); hESCs grew in either optically dense aggregates resembling embryoid bodies or in monolayers of large flattened fibroblast-like cells.

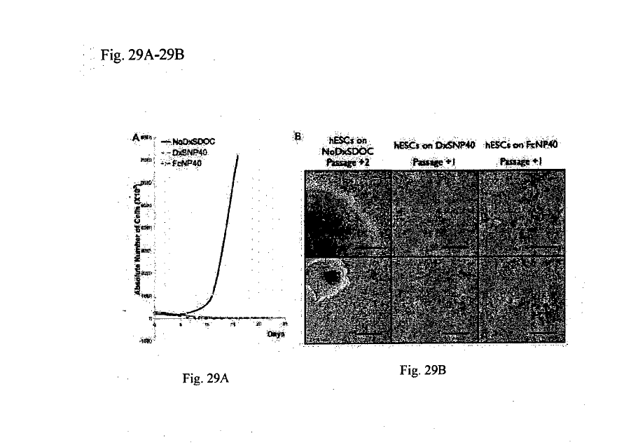

Fibroblasts cultured without addition of macromolecular crowders were lysed with DOC protocol to obtain NoDxSDOC matrices. hESCs cultured on NoDxSDOC matrices proliferated (FIG. 29A), but the hESCs did not maintain the morphology typical of pluripotent hESCs by passage 2 (FIG. 29B). The majority of hESCs grew in optically dense aggregates resembling embryoid bodies, with outgrowths of spontaneously differentiated fibroblast-like cells that were large and flattened.

Fibroblasts that were cultured with either DxS or Fc were lysed with the NP40 protocol to obtain DxSNP40 or FcNP40 matrices. hESCs were also unable to adhere to proliferate on either matrices. Cells that had settled on the bottom of detached easily removed by mechanical disturbances (FIGS. 29A and 29B).

DxSDOC and DxSDOCDOC Matrices Enabled Superior Population Doublings in hESCS Compared to Matrigel.

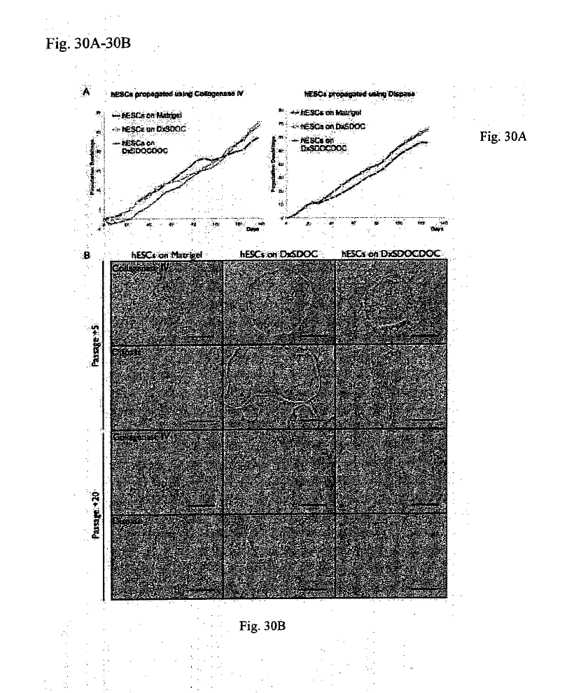

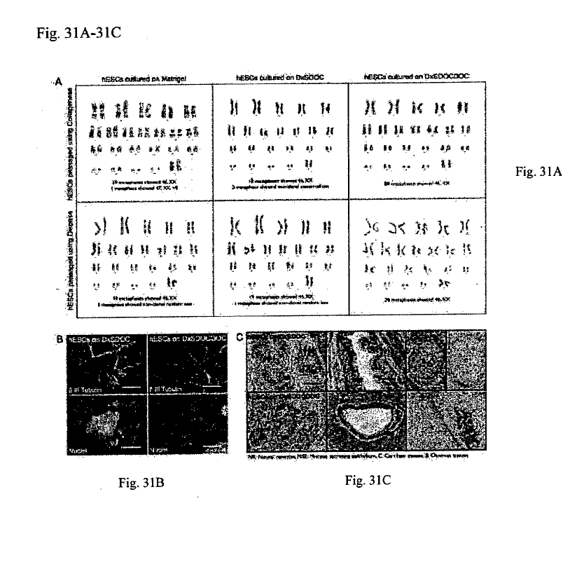

hESCs were propagated on either Matrigel, DxSDOC or DxSDOCDOC matrices for up to 20 passages using either collagenase IV or dispase. Using either enzymes, hESCs cultured on DxSDOC or DxSDOCDOC matrices achieved more population doublings in comparison to the control hESCs on Matrigel (FIG. 30A). Morphologically, DxSDOC- and DxSDOCDOC-propagated hESCs retained their typical small size, high nuclear to cytoplasm ratio and grew in distinct round colonies throughout the 20 passages. In contrast, after 20 passages on Matrigel, more hESC colonies lost their clear borders and larger (differentiated) cells occupied the space between those colonies (red circles, FIG. 30B).

Human ECM-Propagated hESCS Maintain Karyotypic Stability

Because hESCs cultured on Matrigel, DxSDOC or DxSDOCDOC matrices showed differences in their proliferation rates, the karyotype of hESCs cultured under the various experimental conditions was confirmed. DxSDOCDOC-propagated hESCs subcultured using either collagenase IV or dispase retained normal karyotypes for all tested 20 metaphases (FIG. 31A). 18 out of 20 metaphases of collagenase IV-subcultured, DxSDOC-propagated hESCs and 19 out of 20 metaphases of dispase-subcultured, DxSDOC-propagated hESCs were karyotypically normal. 19 out of 20 metaphases of Matrigel-propagated hESCs were normal whether subcultured by either enzymes. This indicates that human fibroblast matrices maintain karyotypic stability in hESCs cultures, which is important in ensuring their future application in transplant therapies.

Human ECM-Propagated hESCs Retain Differentiation Capacity after Long Term Passaging.

As a first step towards characterizing the differentiation potential of human ECM-cultured hESCs, neural differentiation was induced in vitro. The results showed that cells cultured on human fibroblast ECM formed long extensions that stained positive for β III tubulin, a marker for neural differentiation (FIG. 31B). These results indicate that the hESCs were still receptive to neural induction and differentiation and thus indicate their retention of differentiation capabilities.

In a second experiment, the ability of hESCs to differentiate in vivo was also analyzed. hESCs cultured for 18 passages were injected into nude mice and teratoma formation was analyzed. The results showed that teratomas were formed by hESCs propagated on DxSDOC or DxSDOCDOC. The analysis of teratoma tissue sections showed differentiated structures such as neural rosettes, mucus secreting epithelium, cartilage and osseous tissue (FIG. 31C).

Human ECM-Propagated hESCs Maintained Expression of Pluripotency Markers

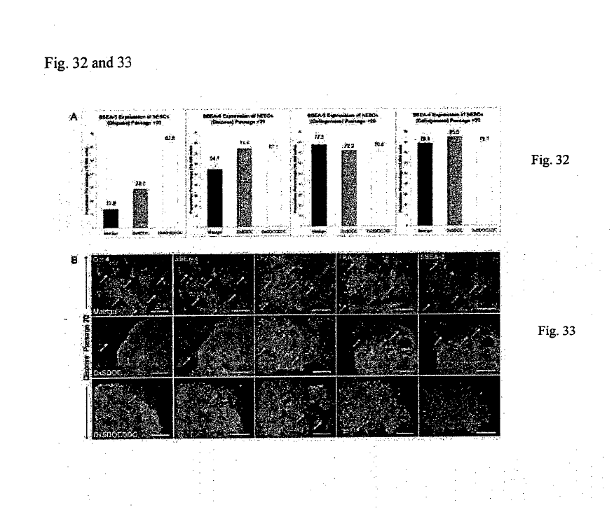

SSEA-3 and SSEA-4 expression of hESCs subcultured using collagenase IV were similar during propagation on Matrigel, DxSDOC or DxSDOCDOC (FIG. 32). However, subculturing using dispase caused a substantially higher SSEA-3 expression of hESCs on DxSDOCDOC matrices at 62.8% in comparison to Matrigel (13.6%) or DxSDOC (28.6%) (FIG. 5A). SSEA-4 expression of DxSDOC- or DxSDOCDOC-cultured hESCs were similar to Matrigel-cultured hESCs (FIG. 32).

Immunofluorescence staining of adherent hESCs for Oct 4, SSEA-4, TRA-1-60, TRA-1-81 and SSEA-3 were in agreement with flow cytometric data and morphology. With increasing passage number, Matrigel-cultured hESCs colonies lost their defined margins and an increasing number of differentiated cells occupied the space between colonies (compare with FIG. 30B). In keeping with their morphology, these cells showed loss of pluripotency marker expression (arrows, FIG. 33). In contrast, hESCs passaged on DxSDOC and DxSDOCDOC showed less cells losing their expression of pluripotency. markers (arrows, FIG. 33). These results suggest a higher degree of maintenance of hESC pluripotency marker expression on human ECM rather than on Matrigel.

Epigenetic Profiling of Matrigel-Propagated hESCs Versus Human ECM-PROPAGATED hESCs

Stem cell-specific DNA methylation patterns are important molecular markers of pluripotency. A prominent example in this context is the methylation status of the OCT4 promoter region, which is unmethylated in hESCs, but methylated in differentiated cells. Deep (454) bisulfite sequencing was used to analyze OCT4 methylation of hESCs cultivated on Matrigel or human fibroblast matrices. The results showed that the OCT4 promoter was unmethylated under all conditions analyzed.

In the next set of experiments Illumina Infinium genome-wide methylation was used analysis to determine the methylation status of 27,578 CpG dinucleotides. This analysis was performed with hESCs cultured on Matrigel, DxSDOC or DxSDOCDOC and generated 3 million data points, with beta values for individual markers ranging from 0 (unmethylated) to 1 (completely methylated). For subsequent analysis, all X-chromosome data was excluded to eliminate the influence of sex-specific X-chromosome methylation differences. Unsupervised hierarchical clustering of the 3 newly obtained methylation profiles together with 14 published profiles from other tissues revealed an high overall similarity in the methylation patterns of defined cell types.

A comparison of methylation patterns between Matrigel and DxSDOC cultures and between Matrigel and DxSDOCDOC cultures revealed a high degree of similarity with Person correlation coefficients exceeding 0.97. However, this analysis also revealed 426 markers that were differentially methylated between Matrigel and DxSDOC cultures and 194 markers that were differentially methylated between Matrigel and DxSDOCDOC cultures. These two groups showed an overlap of 49 markers that were differentially methylated in Matrigel-propagated vs. human ECM-propagated hESCs. The 49 markers represent a diverse group of genes with many different functions. While the functional significance of this observation will have to be analyzed in future studies, it is possible that the differential methylation of certain genes contributes to the superior pluripotency of hESCs cultured on human ECM.

### Discussion

Shown herein is that human ECM bioassembled in vitro can support hESC propagation at least as well as a xenogenic tumor matrix extract. The matrices are generated under macromolecular crowding in order to ensure not only speedy deposition in a short time window of 48 hrs but also with a deposition yield that allows for losses during detergent-based decellularisation and a crosslinking that renders the matrices more resistant to detergent treatment. From a variety of cell types tested we found that the matrices made by a fetal lung fibroblast cell line, WI-38, was most permissive to hESC propagation, whereas matrices generated from human embryonic kidney cell line, 293T, and human neuronally committed teratocarcinoma cell line, NT2, were unable to maintain hESC pluripotent proliferation beyond 12 passages. Wi38 fibroblasts crowded with macromolecules, DxS, were subjected to two different detergent treatments (3 washes versus 6 washes) resulted in two matrices, DxSDOC or DxSDOCDOC. Both, especially DxSDOCDOC, increased the number of population doublings achievable by hESCs, with better retention of undifferentiated morphology, pluripotency marker expression and differentiation potential, compared to Matrigel, DxS increases collagen I deposition in fibroblast cultures by excluding available volume in the culture medium, thereby driving rate-limiting enzymatic steps controlling collagen deposition (Lareu R R et al. 2007a Tissue Eng 13(2): 385-391); Lareu R R et al. 2007b FEBS Letters 581:2709-2714; Chen CZC, et al. 2009b, Br J Pharmacol 158(5): 1196-1209; Peng Y, Raghunath M. 2010 ‘Chapter 5: Learning from nature: emulating macromolecular crowding to drive extracellular matrix enhancement for the creation of connective tissue in vitro’ in Tissue Engineering, ed. Eberl D, IN-TECH, Croatia). Macromolecular crowding also drives the supramolecular assembly of collagen and its template, fibronectin (Kadler K E, et al. 2008 Curr Opin Cell Biol 20(5-24):495-501) (unpublished observation). Both collagen I and fibronectin are major ECM components, acting as two of the main structural scaffolds of ECM, and carry a variety of other ligands that attach covalently or non-covalently (Rosso F et al. 2004 J Cellular Physiol 199(2): 174-180).

DOC and DOCDOC lysis were necessary but sufficient for cell removal. Although detergent treatment led to substantial ECM component loss, it apparently did not compromise ECM functionality nor complexity, in which there was interest because the combination of several factors in certain stoichiometric ratios seems to be crucial for maintaining hESC pluripotency (Ludwig T et al. 2006 Nat Biotechnol 24(2): 185-187). Interestingly, the strongly reduced amounts of collagen I in DxSDOCDOC did not impede hESC propagation and embryoid body formation. However, ECM formed in the absence of DxS, led to a rapid differentiation of hESCs. The need for a macromolecule such as DxS might be due to quantitative and qualitative aspects of macromolecular crowding. Macromolecular crowding stabilises deposited ECM in vitro by increasing enzymatic crosslinking by lysyl oxidase (Lareu R R et al. 2007a Tissue Eng 13(2): 385-391); Lareu R R et al. 2007b FEBS Letters 581:2709-2714; Chen CZC and Raghunath M. 2009a, Fibrogenesis & Tissue Repair 2:7) and by transglutaminase 2. The overall higher degree of enzymatic crosslinking might lead to a greater retention of the ECM and its ligands during the DOC lysis steps and preserves more biochemical cues to hESCs. While the absolute amount of collagen deposition is increased under macromolecular crowding (Lareu R R et al. 2007a Tissue Eng 13(2): 385-391); Lareu R R et al. 2007b FEBS Letters 581:2709-2714) it also will increase the presence of collagen-binding ligands, such as decorin (Aumailley M, Gayraud B. 1998. J Mol Med 76(3-4): 253-265; Schonherr E, Hausser H J. 2000 Develop Immunol 7(2-4): 89-101), and so both amount and total composition of ECM post detergent treatment are increased in comparison to ECM formed in the absence of MMC. Interestingly, mass spectrometry analysis revealed the presence of transglutaminase 2 after DOC and DOCDOC detergent treatments; this enzyme plays a role not only in crosslinking proteins of the ECM, it is also important for apoptosis, cellular differentiation (Griffin M, et al. 2002, Biochem 368(2):377-396) and cell adhesion (Wang Z et al. 2011 Exp Cell Res 317(3):367-381; Nadalutti C et al. 2011 Cell Prolif 44(1):49-58).

With regards to complexity we achieved overlapping and in some instances discrepant results between immunofluorescence and mass spectrometry. While immunofluorescence studies confirmed the presence of heparan sulfate proteoglycans, LTBP-1 and decorin, they did not turn up in mass spectrometric analysis. Immunofluorescence analysis does not require as many processing steps as mass spectrometry, and the multiple processing steps required during mass spectrometric analysis could have led to the loss of components, resulting in the discrepancies reported here. Nevertheless, the overlapping results between the two methods confirmed the presence of collagen I, fibronectin and fibrillin-1. Like collagen I and fibronectin, fibrillin-1 plays dual roles as a structural protein and as a binding partner for several other ligands such as LTBP-1 (Raghunath M et al. 1998 Soc Invest Dermatol 111(4): 559-564). Fibrillins associate into microfibrils and bind to elastin to form the third structural scaffold of ECM (Aumailley M, Gayraud B. 1998. J Mol Med 76(3-4): 253-265). Heparan sulfate proteoglycans are docking points for FGF-2, an important growth factor added into most hESC culture media. Soluble FGF-2 has a notorious short half life in culture medium. However, when bound to ECM, FGF-2 has a prolonged biological action (Rifkin D B, Moscatelli D. 1986, J Cell Biol 109:1-6; Saksela O et al. 1988 J Cell Biol 107: 743-751). Another commonly added growth factor for hESCs is TGFβ-1. This growth factor is stored in its latent form, bound to LTBP-1 (Raghunath M et al. 1998 Soc Invest Dermatol 111(4): 559-564; Taipale J et al. 1996 J Histochem Cytochem 44(8): 875-889), and cells are able to activate this TGFβ-1 latent form upon demand (Annes J P, et al. 2003 J Cell Sci 116(2): 217-224; Chong H, et al. 1999, J Cellular Physiology 178: 275-283). The presence of heparan sulfate proteoglycans and LTBP-1 in the human bioassembled matrices indicates that the can capture and retain growth factors and can condition hESCs during culture. Decorin in DxSDOC and DxSDOCDOC matrices, on the other hand, serves a different role. It captures activated TGFβ-1, thereby regulating TGFβ-1 signaling to the hESCs, hence serving as a form of negative conditioning for hESCs. The combination of both LTBP-1 and decorin in the human matrices would thus help to keep TGFβ-1 signaling to an optimum level for hESCs.

Finally, DOC and DOCDOC treatments might work through differentially removing differentiation-driving cues from such an ECM preferably as compared to matrices generated by NP40, such as DxSNP40 and FcNP40 matrices. The cell type used to generate ECM is also important, as not all cell types can generate pluripotent hESC-compatible ECM, as evident by the differentiation of hESCs on ECM made using human embryonic kidney 293T cells and teratocarcinoma NT2 cells within 12 passages (FIG. 35). The hESCs formed loosely adhering dense aggregates on 293TDOC matrices that may be surrounded by large flattened cells. hESCs cultured on NT2DOC matrices grew in small clusters that often exhibited crater-like morphology, an indication of endodermal differentiation. These small clusters may also be surrounded by large expanses of flattened differentiated cells. These morphological changes, in hESCs were brought about despite the use of mTeSR-1, and so could be the result of the cell-type specific ECM presented to them.

The DxSDOC and DxSDOCDOC matrices thus serve as models of the microenvironment, without the influence of cell-cell interactions, allowing the study, of the role of ECM on hESCs propagation. The complexity of the matrices and the preservation of their native structural arrangements could have enhanced hESC pluripotent proliferation. Mass spectrometric analysis of Matrigel, DxSDOC and DxSDOCDOC matrices showed differences between the murine sarcoma derivative and our human bioassembled matrices. 13 out of 44 protein identifications in DxSDOC matrices were not found in mass spectrometric analysis of Matrigel samples done by Hughes et. al., while 14 out of 29 DxSDOCDOC were not found in Matrigel samples (Hughes C S, et al. 2010, Proteomics 10(9):1886-1890). Differences lie not only in the components found with each ECM; even if a component were present in all three ECM, the component could be a different homolog in Matrigel; 15 proteins (DxSDOC) and 8 proteins (DxSDOCDOC) were homologous in mouse. Several of these homologous proteins are important extracellular matrix proteins: collagens, EMILIN-1, fibrillin-1, fibronectin, tenascin, thrombospondin-1 and vitronectin, which play significant roles in regulating cellular functions. The use of homologous proteins might negatively impact on the regulation of hESC cellular functions, so it would be more advantageous to use human-derived ECM rather than murine sources of ECM.

Matrigel is commonly used for feeder-free culture of hESCs mainly because it is easily available and convenient to use, and most choose to overlook its disadvantageous properties that it is of non-human origins and tumor derived. For the purpose of clinical applications, convenience and easy availability are insufficient justifications for subjecting hESCs to suboptimal conditions, and may impede the progress of hESC culture conditions towards an ideal substrate-medium combination.

More recently, to address the issues of scalability of hESC culture, Corning has launched a new product for hESC culture, Synthemax™-T Surface, compromising a vitronectin-derived peptide covalently linked acrylate polymer surface. This product was launched based on the work of Melkoumian et. al. who described RGD-containing-peptides-linked acrylate surfaces suitable for hESC self-renewal (Melkoumian Z. (2010)). Villa-Diaz et al. have also reported that hESCs can be maintained on synthetic polymer coatings in conditioned medium (Villa-Diaz L G et al. 2010 Nat Biotechnol 28(6):581-583). Interestingly, it was suggested that RGD sequences alone was not the only criteria for successful maintenance of hESCs, as not all RGD-containing peptides was suitable (Melkoumian Z et al. 2008 Nat Biotechnol 28(6): 606-610). This raises the possibility that regions other than the RGD sequences serve critical purposes, possibly serving as binding points for other ligands to bind. Not only does the culture substrate presented to the hESC need to allow for hESC attachment, it has to provide means for presenting other micro environmental cues to the hESCs to maintain their pluripotent proliferation. This implies that for simplified single-component culture substrates, the source of the binding ligands have to be either the culture medium, or the hESC cell line itself (Azrin et. al., 2010). The synthetic polymer coating developed by Villa-Diaz et al. was unable to maintain a hESC line, BG01, in defined culture media, StemPro and mTeSR, while unable to maintain another cell line, H9, in mTeSR. The polymer coating, however, was able to maintain H9 in StemPro for 10 passages. Taken together, these observations support the liklihood that single-component culture substrates can maintain hESC lines selectively and only in conjunction with certain culture media, indicating that simple single component culture substrates are less robust than complex multiple component substrates, and their efficacy easily affected by culture medium or cell line characteristics. Moreover, given the variability between different hESC lines, and even variability of sensitivity to environmental cues within a cell line (Wray J et al. 2010 Biochem Soc Trans 38:1027-1032), the use of simple component substrates could lead to a selection for a subpopulation of hESCs, rather than preserving a myriad of hESCs. In all, although it is tempting to use simple component substrates, complex culture substrates could prove, over long term, to be more stable, as it acts as buffer against several environmental cues coming from culture media or even the cell lines themselves.

As such, development of ECMs that are simple single-component was not sought, but rather use of nature's expert ECM producers, human fibroblasts, to synthesize ECM was used. Although the use of human fibroblasts to produce human ECM might appear to be difficult for scale up, it is possible using automated cell culture systems. Furthermore, because it was understood that the standard culture conditions of human fibroblasts are suboptimal for ECM production, their culture conditions were improved by applying macromolecular crowding to maximize ECM production, before proceeding with cell removal. Moreover, it is important to maximally preserve the native properties of the ECM during the lysis process, as cells detect the composition, geometry and stiffness of their substrates (Daley P W, et al. 2008, J Cell Science 121: 255-264) and ECM properties affect cell fate and differentiation. These aspects are also important for the epigenetic profile of embryonic stem cells. It has been shown recently, that the DNA methylation profile of cultured mouse ESCs is more similar to post-implantation epiblasts than to blastocysts, which has been interpreted to reflect the influence of artificial cell culture conditions (Borgel J, et al. 2010, Nat. Genet. 42(12): 1093-100).

The main drive towards using simple culture substrates was originally a response to the demands for scalable hESC culture. As will be appreciated by those of skill in the art, the production of the human matrices presented here could be scaled up easily using automated cell culture systems. In addition, although the human matrices are not xeno-free due to the use of FBS, this work shows that feeder-free human fibroblast-derived ECM can be used for hESC propagation. Currently, the only xeno-free substitute for FBS is human serum, and the widespread application of human serum in cell culture is impeded by its limited supply and varying quality. Since only 0.5% FBS is needed in the production of the human matrices, less human serum would be needed. Moreover, because of the supplementation with DxS, FBS requirement was reduced to only 0.5%, hence, it is believed the substitution of FBS with human serum will not affect the performance of hESCs on these future matrices.

### CONCLUSION

Culture of hESCs on human bioassembled ECM, DxSDOC or DxSDOCDOC, increased their achievable number of population doublings while retaining their pluripotency, differentiation potential and as well as their karyotypic and epigenetic stability. Supplementation of DxS during fibroblast culture to produce the human ECMs proved to be crucial for long term pluripotent hESC culture, while concurrently reducing the amount of time and serum required. These complex ECMs contained human native homologs of its constituents and showed preservation of inter-protein interactions, and thus, would serve as a biomimetic human model of the microenvironment for hESCs. As such, these human ECM provides an opportunity to study the effects of the human ECM microenvironment on hESCs.

### Example 5

### Stem Cells (hMSCs) Through Application of Excluded Volume Effect (EVE)

Human bone marrow-derived MSCs have been utilized in cell-based approaches for in vivo adipose tissue engineering to generate novel clinical modalities for autologous repair, reconstruction of fat tissue defects and development of metabolic syndrome bioassays. Currently, the use of hMSCs is limited by the attenuation of their adipogenic differentiation potential following extensive ex vivo expansion ((1) Banfi, A. et al., Exp. Hematol. 28:707-715 (2000)).

As the extracellular matrix is a crowded environment, it was hypothesized that the excluded volume effects (EVE) may be able to generate novel tissue culture conditions that more closely resembles in vivo physiological conditions ((2) Ellis, J. R., Trends in Biochem. Sci. 26:597-603 (2001)). These conditions may in turn augment adipogenic differentiations of MSCs.

### Materials and Methods

EVE exerted by a cocktail of macromolecules Ficoll™ 70 (37.5 mg/ml) and Ficoll™ 400 (25 mg/ml), was tested for its ability to augment adipogenesis of hMSCs at a; late passage (p8) with a commercial differentiation kit.

### Results

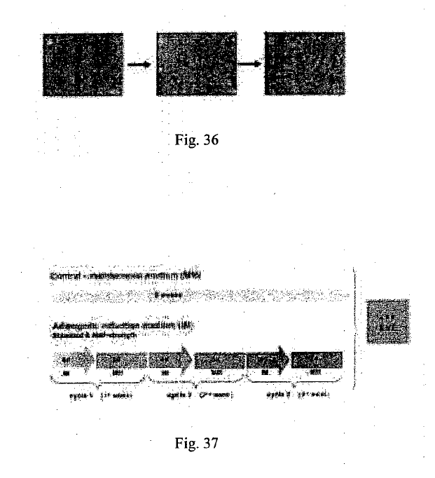

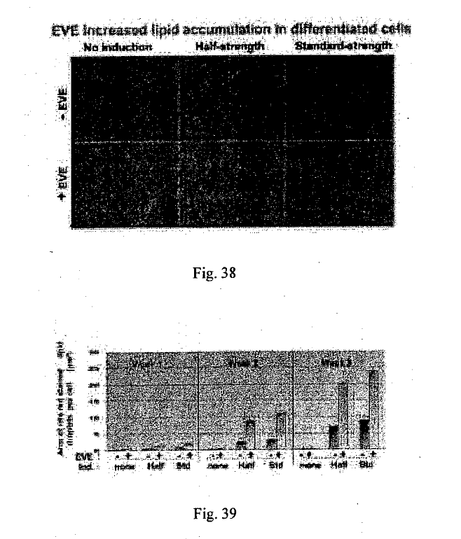

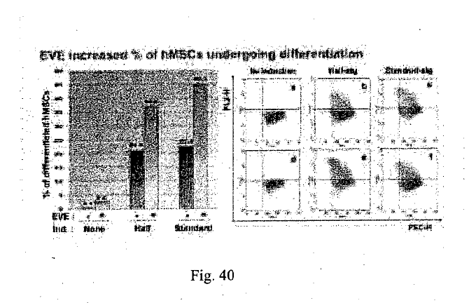

See FIGS. 36-40.

EVE augmented adipogenesis by increasing amount of lipid accumulated with the cell, and increasing percentage of cells that undergoes adipogenesfs:

EVE enabled a more effective use of less induction ingredients.

Ficoll is an inert crowder and did not cause spontaneous adipogenesis.

More cells with desired phenotype were generated for tissue engineering via EVE.

### Example 6

### During Adipogenesis of Human Mesenchymal Stem Cells—Tool for Translational Tissue Engineering

The physiological microenvironment or “niche” where human bone marrow mesenchymal stem cells (MSCs) reside is crowded due to the dense arrays of extracellular matrix (ECM) macromolecules and solutes ((1) Zimmerman, S. B. and Minton, A. P. (1993)).

Taken together, these macromolecules occupy a significant fraction (typically 20-30%) of the total volume, creating steric exclusions effect with considerable energetic consequences which influences the equilibria and rates of biochemical reactions dependent on non-covalent associations and/or conformational changes, such as protein and nucleic acid synthesis, intermediary metabolism, cell signaling and gene expression ((2) Minton, A. P. (1981) Biochemistry 20:4821-6). In contrast, contemporary culture systems are characterized by the absence of crowding where thin cell layers are flooded with aqueous medium which result in the dilution of signaling molecules and efficient deposition of the extracellular matrix (ECM) ((3) Lareu, R. R., et al., (2007) Tissue Eng. 13, 385-391).

The ex vivo differentiation of MSCs is modulated by defined soluble factors and cell-ECM interactions; both which are affected by macromolecular association kinetics during ligand diffusion, ligand receptor binding and conformations of cell surface receptors. It was hypothesized that the addition of a neutral macromolecule cocktail to effect macromolecular crowding (MMC) may be able to generate a more physiological microenvironment ex vivo which could function to enhance the deposition of a lineage-specific ECM; this may in turn augment adipogenic differentiation of MSCs.

### Results

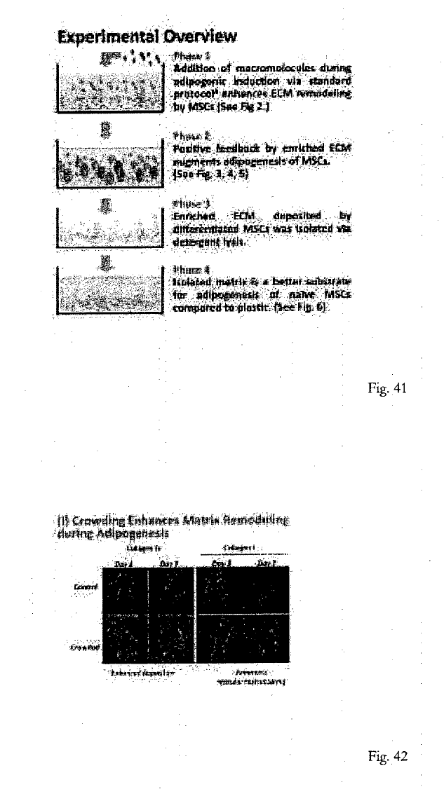

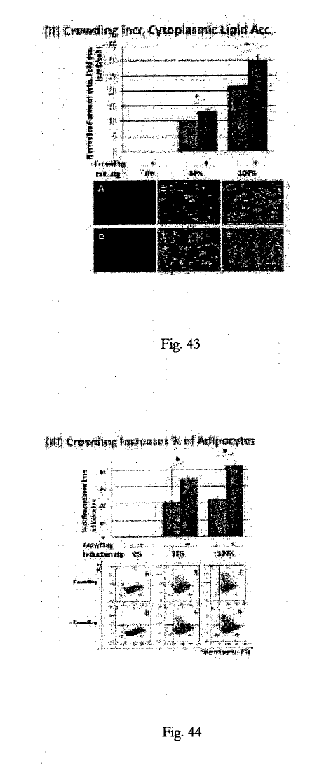

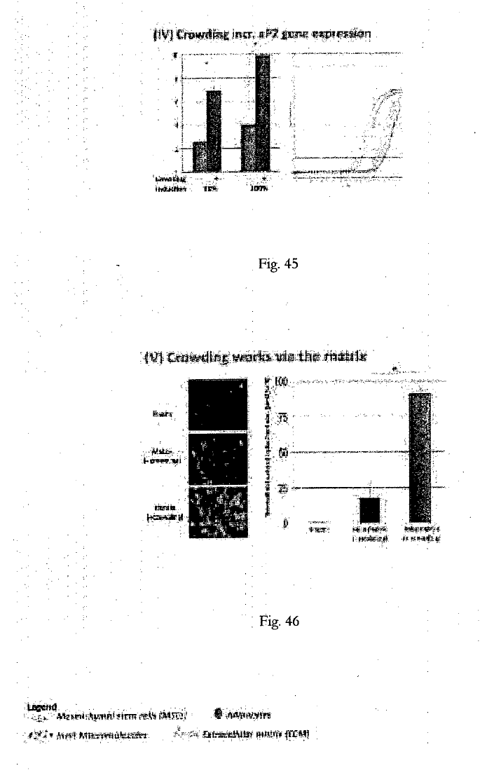

See FIGS. 41-45.

Crowding augments the process of matrix remodeling during adipogenesis.

Crowding augmented adipogenesis by increasing amount of cytoplasmic lipid accumulated within the cell, increasing percentage of cells that undergoes adipogenesis, and increasing the expression of key lipogenic gene (aP2).

Matrix deposited under crowding serves as a better substrate for adipogenic differentiation compared to plastic or matrix deposited in the absence of crowding.

Crowding augments adipogenesis by enhancing the deposition and remodeling of a lineage-specific matrix (niche).

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by a preferred embodiment, modification and variation of the invention herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of culturing stem cells comprising:
a) contacting the stem cells with one or more carbohydrate-based macromolecules, thereby producing a cell culture, and
b) maintaining the cell culture under conditions in which the stem cells proliferate,
thereby culturing the steprn cells.

2. The method of claim 1 wherein the concentration of each of the one or more carbohydrate-based macromolecules is from about 2.5 mg/ml to about 100 mg/ml.

3. The method of claim 2 wherein the one or more carbohydrate-based macromolecules has a molecular weight from about 50 kDa to about 1000 kDa.

4. The method of claim 3 wherein the one or more carbohydrate-based macromolecules is a polymer of glucose, sucrose or a combination thereof.

5. The method of claim 4 wherein the polymer is Ficoll™ 70, Ficoll™ 400, polyvinyl pyrrolidone, dextran, dextran sulfate, polystyrene sulfonate, pullulan or a combination thereof.

6. The method of claim 1 further comprising contacting the stem cells with a macromolecule having a negative or neutral charge.

7. The method of claim 6 wherein the macromolecule is polyvinyl pyrrolidone.

8. The method of claim 1 wherein the stem cells are contacted with the one or more macromolecules at a seeding density of about 500 cell/cm² to 30,000 cells/cm².

9. The method of claim 1 wherein the stem cells are mesenchymal stem cells, hematopoietic stem cells, induced pluripotent stem cells, embryonic stem cells, fibrocytes or a combination thereof.

10. The method of claim 9 wherein the cells are human cells.

11. The method of claim 1 wherein the stem cells are contacted with a mixture of carbohydrate-based macromolecules comprising Ficoll™ 70 and Ficoll™ 400 and the stem cells are mesenchymal stem cells or fibrocytes.

12. The method of claim 11 wherein the Ficoll™ 70 is at a concentration range of from about 2.5 mg/ml to about 100 mg/ml, and the Ficoll™ 400 is at a concentration range of from about 2.5 mg/ml to about 100 mg/ml.

13. The method of claim 12 wherein the Ficoll™ 70 is at a concentration of about 37.5 mg/ml and the Ficoll™ 400 is at a concentration of about 25 mg/ml.

14. The method of claim 1 further comprising inducing the stem cells to differentiate.

15. The method of claim 14 wherein the stem cells are induced to differentiate by contacting the stem cells with one or more agents that induce differentiation.

16. The method of claim 15 wherein the stem cells differentiate into adipocytes, osteoblasts, chondrocytes, neurons, cardiomyocytes, myocytes or endothelial cells,

17. A method of producing an extracellular matrix comprising
a) contacting fibroblasts, mesenchymal stem cells or a combination thereof with one or more carbohydrate-based macromolecules, thereby producing a cell culture, and
b) maintaining the cell culture under conditions in which the fibroblasts or mesemchyrnal stem cells or combination thereof proliferate and produce an extracellular matrix.

18. The method of claim 17 wherein the concentration of each of the one or more macromolecules is from about 2.5 m/ml to about 100 mg/ml.

19. The method of claim 18 wherein the one or more carbohydrate-based macromolecule has a molecular weight from about 50 kDa to about 1000 kDa.

20. The method of claim 17 wherein the one or more carbohydrate-based macromolecule is a polymer of glucose, sucrose or a combination thereof.

21. The method of claim 20 wherein the polymer is Ficoll™ 70, Ficoll™ 400, dextran sulfate or a combination thereof.

22. The method of claim 17 wherein the fibroblasts are human fibroblasts.

23. The method of claim 22 wherein the human fibroblasts are embryonic human fibroblasts, fetal human fibroblasts, human fibroblasts derived from mesenchymal stem cells, induced pluripotent stem cells, human embryonic stem cells or a combination thereof.

24. The method of claim 23 wherein the human fibroblasts are Wi-38 fibroblasts, IMR-90 cells, 293 cells or a combination thereof.

25. The method of claim 17 further comprising c) contacting the cell culture with one or more agents that lyse the cells, thereby producing a cell-free extracellular matrix.

26. The method of claim 25 wherein the one or more agents is deoxycholate, NP40 DNase or a combination thereof.

27. A cell-free extracellular matrix produced by the method of claim 25.

28. A method of culturing stem cells comprising
a) contacting the stem cells with the cell-free extracellular matrix of claim 22, thereby producing a stem cell culture, and
b) maintaining the stem cell culture under conditions in which the stem cells proliferate,
thereby culturing the stem cells.

29. A kit comprising a cell-free extracellular matrix produced by the method of claim 25 and instructions for use thereof.

30. The kit of claim 29 further comprising plates coated with the cell-free extracellular matrix.
